(19) United States
(12) Patent Application Publication
(10) Pub. No.: US 2021/0151155 A1
(43) Pub. Date: May 20, 2021

(54) METHODS AND SYSTEMS FOR MANAGING DISTRIBUTION AND TREATMENT OF A FOOD ALLERGY ORAL IMMUNOTHERAPY DRUG

(71) Applicant: Aimmune Therapeutics, Inc., Brisbane, CA (US)

(72) Inventors: Adrian DANA, Brisbane, CA (US); Daniel ADELMAN, Redwood City, CA (US)

(21) Appl. No.: 16/953,183

(22) Filed: Nov. 19, 2020

Related U.S. Application Data
(60) Provisional application No. 63/056,492, filed on Jul. 24, 2020.
(60) Provisional application No. 62/938,180, filed on Nov. 20, 2019.

(51) Int. Cl. G16H 20/10; G16H 10/20; G16H 10/60; G16H 40/67; G16H 50/20; G16H 50/70; G16H 80/00

(52) U.S. Cl. CPC G16H 20/10; G16H 10/20; G16H 10/60; G16H 80/00; G16H 50/20; G16H 50/70; G16H 40/67

(57) ABSTRACT

Methods and systems for managing the distribution of a drug comprising a food allergen are described herein. Exemplary methods allow for the enrollment of distributors, pharmacies, healthcare settings, healthcare providers, and/or patients in a drug distribution management database for the drug, wherein distribution or provision of the drug is contingent on ensuring enrollment of one or more of the entities in the drug distribution management database. Other methods utilizing the drug distribution management database are also described.

### CROSS-REFERENCE TO RELATED APPLICATIONS

This application claims the priority benefit of U.S. Provisional Patent Application Ser. No. 62/938,180, filed Nov. 20, 2019; and U.S. Provisional Patent Application Ser. No. 63/056,492, filed Jul. 24, 2020; the contents of each which are incorporated herein by reference in their entirety.

### FIELD OF THE INVENTION

Computer-implemented methods for managing the distribution of a drug comprising a food allergen are described herein. Electronic systems and devices for implementing such methods are also described. Further provided are methods of treating a patient for a food allergy by oral immunotherapy that include managing the distribution of a drug comprising a food allergen, and related electronic devices and systems.

### BACKGROUND OF THE INVENTION

Food allergies are common, life-long conditions that often appear in early childhood. Peanut allergy is the most common food allergy, affecting approximately 2% of children in the United States, and approximately 80% of children with peanut allergy will remain peanut allergic throughout their lifetime. Accidental exposures to peanuts resulting in allergic reactions are common due to the presence of peanut protein in many foods and the numerous possibilities for contamination. Peanut allergy reactions are the most common cause of severe and potentially life-threatening allergic reactions and fatal anaphylaxis from food.

A recently develop pharmaceutical composition, PALFORZIA™ [Peanut (Arachis hypogaea) Allergen Powder-dnfp] containing peanut flour formulated with excipients and packaged into unit doses at carefully controlled dose levels has been developed for treating peanut allergy in patients using oral immunotherapy. See, e.g., U.S. Pat. No. 9,198,869; U.S. Pat. No. 9,492,535; U.S. Pat. No. 10,086,068; and U.S. Pat. No. 10,449,118; each of which is incorporated herein by reference for all purposes. The drug is formulated as an oral powder containing characterized peanut allergens that is administered once daily by mixing the powder with food. The pharmaceutical composition is used as oral immunotherapy to reduce the incidence and severity of allergic reactions, including anaphylaxis, after accidental exposure to peanut in patients aged 4 to 17 years with a confirmed diagnosis of peanut allergy. Other oral immunotherapy drug compositions containing formulated protein allergen powders are also being developed for other food allergies, such as egg white allergy or various tree nut allergies. See, e.g., WO 2016/033094 and WO 2018/132733, each of which is incorporated herein by reference for all purposes.

The dosing regimen of the oral immunotherapy drug is generally administered in 3 phases: initial dose escalation, up-dosing, and maintenance. The initial dose escalation is administered as a series of small but increasing doses of the drug as the patient is monitored for an allergic response. The up-dossing phase generally includes the daily administration of a set dose (i.e., level) of the drug for a period of time before a higher dose of the drug is administered to the patient for a period. After the up-dosing phase, a set maintenance dose of the drug is administered to the patient.

### SUMMARY OF THE INVENTION

Computer-implemented methods for authorizing the distribution of a drug comprising a food allergen for oral immunotherapy are described herein. Further described are electronic devices that include a memory storing one or more programs for implementing drug distribution management methods, computer-readable media storing one or more programs for implementing drug distribution management methods, and computer programs that, when executed by a computer, implement drug distribution methods. Methods of distributing the drug or treating a patient with the drug are also described herein.

In some embodiments, a computer-implemented method for authorizing the provision of a drug comprising a food allergen to a healthcare setting, comprises: (a) receiving, at a first electronic device communicatively coupled to a drug distribution management database for the drug and from a second electronic device associated with a pharmacy enrolled in the drug distribution management database, a healthcare setting enrollment verification request comprising healthcare setting information for a healthcare setting and a patient enrollment verification request comprising patient information for a patient; (b) generating, at the first electronic device, a healthcare setting enrollment status, comprising (i) accessing the drug distribution management database, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generating, at the first electronic device, a patient enrollment status, comprising (i) accessing the drug distribution management database, (ii) comparing the patient information in the patient enrollment verification request to enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule. In some embodiments, step (a) further comprises receiving, at the first electronic device, from the second electronic device, a healthcare provider enrollment verification request comprising healthcare provider information for a healthcare provider; wherein the method further comprises a step of generating, at the first electronic device, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; wherein step (d) further comprises sending, to the second electronic device, the healthcare provider enrollment status, wherein receipt of each of a positive healthcare setting enrollment status, a positive patient enrollment status, and a positive healthcare provider enrollment status by the pharmacy authorizes the pharmacy to provide the healthcare setting the initial dose escalation kit associated with the patient.

In some embodiments, a computer-implemented method for authorizing the provision of a drug comprising a food allergen to a healthcare setting or a patient, comprises: (a) receiving, at a first electronic device communicatively coupled to a drug distribution management database for the drug and from a second electronic device associated with a pharmacy enrolled in the drug distribution management database, a healthcare setting enrollment verification request comprising healthcare setting information for a healthcare setting and a patient enrollment verification request comprising patient information for a patient; (b) generating, at the first electronic device, a healthcare setting enrollment status, comprising (i) accessing the drug distribution management database, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generating, at the first electronic device, a patient enrollment status, comprising (i) accessing the drug distribution management database, (ii) comparing the patient information in the patient enrollment verification request to enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting or the patient a plurality of doses of the drug at a single treatment dose level according to an oral immunotherapy dosing schedule. In some embodiments, step (a) further comprises receiving, at the first electronic device, from the second electronic device, a healthcare provider enrollment verification request comprising healthcare provider information for a healthcare provider; wherein the method further comprises generating, at the first electronic device, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; wherein step (d) further comprises sending, to the second electronic device, the healthcare provider enrollment status, wherein receipt of each of a positive healthcare setting enrollment status, a positive patient enrollment status, and a positive healthcare provider enrollment status by the pharmacy authorizes the pharmacy to provide to the healthcare setting or the patient the plurality of doses of the drug at a single treatment dose level. In some embodiments, the method further comprises repeating steps (a)-(d) at a plurality of different treatment dose levels, wherein the pharmacy is not authorized to simultaneously provide the drug at more than one treatment dose level for the patient.

In some embodiments, a computer-implemented method for authorizing the provision of a drug comprising a food allergen to a patient, comprises: (a) receiving, at a first electronic device communicatively coupled to a drug distribution management database for the drug and from a second electronic device associated with a pharmacy enrolled in the drug distribution management database, a healthcare provider enrollment verification request comprising healthcare provider information for a healthcare provider and a patient enrollment verification request comprising patient information for a patient; (b) generating, at the first electronic device, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; (c) generating, at the first electronic device, a patient enrollment status, comprising (i) accessing the drug distribution management database, (ii) comparing the patient information in the patient enrollment verification request to enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare provider enrollment status and the patient enrollment status, wherein receipt of a positive healthcare provider enrollment status and a positive patient enrollment status by the pharmacy authorizes the pharmacy to provide to the healthcare provider or the patient a plurality of doses of the drug at a single treatment dose level according to an oral immunotherapy dosing schedule. In some embodiments, the method further comprises repeating steps (a)-(d) at a plurality of different treatment dose levels, wherein the pharmacy is not authorized to simultaneously provide the drug at more than one treatment dose level for the patient.

In some embodiments of the methods described above, the method further comprises receiving, by the first electronic device, a patient enrollment request comprising patient information for the patient; and adding, at the first electronic device, an entry comprising the patient information into the drug distribution management database to enroll the patient in the drug distribution management database.

In some embodiments of the methods described above, the method further comprises receiving, at the first electronic device, a pharmacy enrollment request comprising pharmacy information for the pharmacy; and adding, at the first electronic device, an entry comprising the pharmacy information to the drug distribution management database to enroll the pharmacy in the drug distribution management database.

In some embodiments of the methods described above, the method further comprises receiving, at the first electronic device, a healthcare setting enrollment request comprising healthcare setting information for the healthcare setting; and adding, at the first electronic device, an entry comprising the healthcare setting information to the drug distribution management database to enroll the healthcare setting in the drug distribution management database.

In some embodiments of the methods described above, the method further comprises receiving, at the first electronic device, a healthcare provider enrollment request comprising healthcare provider information for the healthcare provider; and adding, at the first electronic device, an entry comprising the healthcare provider information to the drug distribution management database to enroll the healthcare provider in the drug distribution management database.

Also described herein is a computer program product comprising instructions which, when the program is executed by a computer, causes the computer to carry out the steps of any one of the methods described above.

In some embodiments, an electronic device comprises one or more processors; a memory configured to store a drug distribution management database for a drug comprising a food allergen, the drug distribution management database comprising entries for enrolled patients comprising enrolled patient information, entries for enrolled healthcare settings comprising enrolled healthcare setting information, and entries for enrolled pharmacies comprising enrolled pharmacy information; and one or more programs stored on the memory configured to be executed by the one or more processors, the one or more programs including instructions for: (a) receiving, from a second electronic device associated with a pharmacy enrolled in the drug distribution management database, a healthcare setting enrollment verification request comprising healthcare setting information for a healthcare setting and a patient enrollment verification request comprising patient information for a patient; (b) generating, by the one or more processors, a healthcare setting enrollment status, comprising (i) accessing the drug distribution management database, (ii) comparing the healthcare setting information in the healthcare setting enrollment verification request to the healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generating, by the one or more processors, a patient enrollment status, comprising (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule. In some embodiments, the drug distribution management database further comprises entries for enrolled healthcare providers comprising enrolled healthcare provider information; wherein the instructions of (a) further comprises receiving, at the first electronic device, from the second electronic device, a healthcare provider enrollment verification request comprising healthcare provider information for a healthcare provider; wherein the one or more programs further include instructions for generating, at the first electronic device, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; wherein the instructions of (d) further comprise sending, to the second electronic device, the healthcare provider enrollment status, wherein receipt of each of a positive healthcare setting enrollment status, a positive patient enrollment status, and a positive healthcare provider enrollment status by the pharmacy authorizes the pharmacy to provide the healthcare setting the initial dose escalation kit associated with the patient.

In some embodiments, an electronic device comprises one or more processors; a memory configured to store a drug distribution management database for a drug comprising a food allergen, the drug distribution management database comprising entries for enrolled patients comprising enrolled patient information, entries for enrolled healthcare providers comprising enrolled healthcare provider information, and entries for enrolled pharmacies comprising enrolled pharmacy information; and one or more programs stored on the memory configured to be executed by the one or more processors, the one or more programs including instructions for; (a) receiving, from a second electronic device associated with a pharmacy enrolled in the drug distribution management database, a healthcare provider enrollment verification request comprising healthcare provider information for a healthcare provider and a patient enrollment verification request comprising patient information for a patient; (b) generating, at the first electronic device, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; (c) generating, at the first electronic device, a patient enrollment status, comprising (i) accessing the drug distribution management database, (ii) comparing the patient information in the patient enrollment verification request to enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare provider enrollment status and the patient enrollment status, wherein receipt of a positive healthcare provider enrollment status and a positive patient enrollment status by the pharmacy authorizes the pharmacy to provide to the healthcare provider or the patient a plurality of doses of the drug at a single treatment dose level according to an oral immunotherapy dosing schedule.

In some embodiments, an electronic device comprises one or more processors; a memory configured to store a drug distribution management database for a drug comprising a food allergen, the drug distribution management database comprising entries for enrolled patients comprising enrolled patient information, entries for enrolled healthcare settings comprising enrolled healthcare setting information, and entries for enrolled pharmacies comprising enrolled pharmacy information; and one or more programs stored on the memory configured to be executed by the one or more processors, the one or more programs including instructions for: (a) receiving, from a second electronic device associated with a pharmacy enrolled in the drug distribution management database, a healthcare setting enrollment verification request comprising healthcare setting information for a healthcare setting and a patient enrollment verification request comprising patient information for a patient; (b) generating, by the one or more processors, a healthcare setting enrollment status, comprising (i) accessing the drug distribution management database, (ii) comparing the healthcare setting information in the healthcare setting enrollment verification request to the healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generating, by the one or more processors, a patient enrollment status, comprising (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting or the patient a plurality of doses of the drug at a single treatment dose level according to an oral immunotherapy dosing schedule. In some embodiments, the one or more programs further including instructions for repeating steps (a)-(d) at a plurality of different treatment dose levels, wherein the pharmacy is not authorized to simultaneously provide the drug at more than one treatment dose level for the patient. In some embodiments, the drug distribution management database further comprises entries for enrolled healthcare providers comprising enrolled healthcare provider information; wherein the instructions of (a) further comprise receiving, from the second electronic device associated with the pharmacy enrolled in the drug distribution management database, a healthcare provider enrollment verification request comprising healthcare provider information for a healthcare provider; wherein the one or more programs further include instructions for generating, by the one or more processors, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; wherein the instructions of (d) further comprise sending, to the second electronic device, the healthcare provider enrollment status, wherein receipt of each of a positive healthcare setting enrollment status, a positive patient enrollment status, and a positive healthcare provider enrollment status by the pharmacy authorizes the pharmacy to provide to the healthcare setting, the healthcare provider, or the patient the plurality of doses of the drug at the single treatment dose level.

In some embodiments of any one of the electronic devices described above, the one or more programs further including instructions for: receiving, by the one or more processors, a patient enrollment request comprising patient information for the patient; and adding, by the one or more processors, an entry comprising the patient information into the drug distribution management database to enroll the patient in the drug distribution management database.

In some embodiments of any one of the electronic devices described above, the one or more programs further including instructions for: receiving, by the one or more processors, a pharmacy enrollment request comprising pharmacy information for the pharmacy; and adding, by the one or more processors, an entry comprising the pharmacy information to the drug distribution management database to enroll the pharmacy in the drug distribution management database.

In some embodiments of any one of the electronic devices described above, the one or more programs further including instructions for: receiving, by the one or more processors, a healthcare setting enrollment request comprising healthcare setting information for the healthcare setting; and adding, by the one or more processors, an entry comprising the healthcare setting information to the drug distribution management database to enroll the healthcare setting in the drug distribution management database.

In some embodiments of any one of the electronic devices described above, the one or more programs further including instructions for: receiving, by the one or more processors, a healthcare provider enrollment request comprising healthcare provider information for the healthcare provider; and adding, by the one or more processors, an entry comprising the healthcare provider information to the drug distribution management database to enroll the healthcare provider in the drug distribution management database.

In some embodiments, a non-transitory computer-readable storage medium storing one or more programs comprising instructions, which when executed by one or more processors of an electronic device, cause the electronic device to: (a) receive, from a second electronic device associated with a pharmacy enrolled in a drug distribution management database for a drug comprising a food allergen, a healthcare setting enrollment verification request comprising healthcare setting information for a healthcare setting and a patient enrollment verification request comprising patient information for a patient; (b) generate, by the one or more processors, a healthcare setting enrollment status by (i) accessing the drug distribution management database, (ii) comparing the healthcare setting information in the healthcare setting enrollment verification request to the healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generate, by the one or more processors, a patient enrollment status by (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) send, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule. In some embodiments, step (a) further comprises receiving, from the second electronic device, a healthcare provider enrollment verification request comprising healthcare provider information for a healthcare provider; wherein the instructions, which when executed by the one or more processors of the electronic device, further cause the electronic device to generate, by the one or more processors, the healthcare provider enrollment status by (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; wherein step (d) further comprises sending, to the second electronic device, the healthcare provider enrollment status, wherein receipt of each of a positive healthcare setting enrollment status, a positive patient enrollment status, and a positive healthcare provider enrollment status by the pharmacy authorizes the pharmacy to provide to the healthcare setting the initial dose escalation kit associated with the patient.

In some embodiments, a non-transitory computer-readable storage medium storing one or more programs comprising instructions, which when executed by one or more processors of an electronic device, cause the electronic device to: (a) receive, from a second electronic device associated with a pharmacy enrolled in a drug distribution management database for a drug comprising a food allergen, a healthcare provider enrollment verification request comprising healthcare provider information for a healthcare provider and a patient enrollment verification request comprising patient information for a patient; (b) generate, by the one or more processors, a healthcare provider enrollment status by (i) accessing the drug distribution management database, (ii) comparing the healthcare provider information in the healthcare provider enrollment verification request to the healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; (c) generate, by the one or more processors, a patient enrollment status by (i) accessing the drug distribution management database, (ii) comparing the patient information in the patient enrollment verification request to the enrolled patient information in the drug distribution management database, and (iii) obtaining a positive patient enrollment status by identifying a match between the patient information in the healthcare setting enrollment verification request and the patient information in the drug distribution management database, or obtaining a negative patient enrollment status if no match between the patient information in the patient enrollment verification request and the patient information in the drug distribution management database is identified; and (d) send, to the second electronic device, the healthcare provider enrollment status and the patient enrollment status, wherein receipt of a positive healthcare provider enrollment status and a positive patient enrollment status by the pharmacy authorizes the pharmacy to provide to the healthcare provider or the patient a plurality of doses of the drug at a single treatment dose level according to an oral immunotherapy dosing schedule.

In some embodiments, a non-transitory computer-readable storage medium storing one or more programs comprising instructions, which when executed by one or more processors of an electronic device, cause the electronic device to: (a) receive, from a second electronic device associated with a pharmacy enrolled in a drug distribution management database for a drug comprising a food allergen, a healthcare setting enrollment verification request comprising healthcare setting information for a healthcare setting and a patient enrollment verification request comprising patient information for a patient; (b) generate, by the one or more processors, a healthcare setting enrollment status by (i) accessing the drug distribution management database, (ii) comparing the healthcare setting information in the healthcare setting enrollment verification request to the healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generate, by the one or more processors, a patient enrollment status by (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) send, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting or the patient a plurality of doses of the drug at a single treatment dose level according to an oral immunotherapy dosing schedule. In some embodiments, (a) further comprise receiving, from the second electronic device associated with the pharmacy enrolled in the drug distribution management database, a healthcare provider enrollment verification request comprising healthcare provider information for a healthcare provider; wherein the instructions, which when executed by the one or more processors of the electronic device, further cause the electronic device to generate, by the one or more processors, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; wherein (d) further comprises sending, to the second electronic device, the healthcare provider enrollment status, wherein receipt of each of a positive healthcare setting enrollment status, a positive patient enrollment status, and a positive healthcare provider enrollment status by the pharmacy authorizes the pharmacy to provide to the healthcare setting, healthcare provider, or the patient the plurality of doses of the drug at the single treatment dose level. In some embodiments, the instructions, which when executed by the one or more processors of the electronic device, further cause the electronic device to repeat steps (a)-(d) at a plurality of different treatment dose levels, wherein the pharmacy is not authorized to simultaneously provide the drug at more than one treatment dose level for the patient.

In some embodiments of any one of the storage media described above, the instructions, which when executed by the one or more processors of the electronic device, further cause the electronic device to: receive, by the one or more processors, a patient enrollment request comprising patient information for the patient; and add, by the one or more processors, an entry comprising the patient information into the drug distribution management database to enroll the patient in the drug distribution management database.

In some embodiments of any one of the storage media described above, the instructions, which when executed by the one or more processors of the electronic device, further cause the electronic device to: receive, by the one or more processors, a pharmacy enrollment request comprising pharmacy information for the pharmacy; and add, by the one or more processors, an entry comprising the pharmacy information to the drug distribution management database to enroll the pharmacy in the drug distribution management database.

In some embodiments of any one of the storage media described above, the instructions, which when executed by the one or more processors of the electronic device, further cause the electronic device to: receive, by the one or more processors, a healthcare setting enrollment request comprising healthcare setting information for the healthcare setting; and add, by the one or more processors, an entry comprising the healthcare setting information to the drug distribution management database to enroll the healthcare setting in the drug distribution management database.

In some embodiments of any one of the storage media described above, the instructions, which when executed by the one or more processors of the electronic device, further cause the electronic device to: receive, by the one or more processors, a healthcare provider enrollment request comprising healthcare provider information for the healthcare provider; and add, by the one or more processors, an entry comprising the healthcare provider information to the drug distribution management database to enroll the healthcare provider in the drug distribution management database.

In some embodiments, a computer-implemented method for authorizing the provision of a drug comprising a food allergen to a pharmacy, comprises (a) receiving, at a first electronic device communicatively coupled to a drug distribution management database for the drug and from a second electronic device associated with a distributor enrolled in the drug distribution management database, a pharmacy enrollment verification request comprising pharmacy information for a pharmacy; (b) generating, at the first electronic device, a pharmacy enrollment status, comprising (i) accessing the drug distribution management database, (ii) comparing the pharmacy information in the pharmacy enrollment verification request to enrolled pharmacy information in the drug distribution management database, and (iii) obtaining a positive pharmacy enrollment status by identifying a match between the pharmacy information in the pharmacy enrollment verification request and the pharmacy information in the drug distribution management database, or obtaining a negative pharmacy enrollment status if no match between the pharmacy information in the pharmacy enrollment verification request and the pharmacy information in the drug distribution management database is identified; and (c) sending, to the second electronic device, the pharmacy enrollment status, wherein receipt of a positive pharmacy enrollment status authorizes the distributor to provide the drug to the pharmacy. In some embodiments, the method further comprises receiving, by the first electronic device, a pharmacy enrollment request comprising pharmacy information for the pharmacy; and adding, at the first electronic device, an entry comprising the pharmacy information into the drug distribution management database to enroll the pharmacy in the drug distribution management database. In some embodiments, the method further comprises receiving, by the first electronic device, a distributor enrollment request comprising distributor information for the distributor; and adding, at the first electronic device, an entry comprising the distributor information into the drug distribution management database to enroll the distributor in the drug distribution management database. In some embodiments, a computer program product comprising instructions which, when the program is executed by a computer, causes the computer to carry out the steps of the method.

In some embodiments, an electronic device comprises one or more processors; a memory configured to store a drug distribution management database for a drug comprising a food allergen, the drug distribution management database comprising entries for enrolled distributors comprising enrolled distributor information, and entries for enrolled pharmacies comprising enrolled pharmacy information; and one or more programs stored on the memory configured to be executed by the one or more processors, the one or more programs including instructions for: (a) receiving, by the one or more processors, a pharmacy enrollment verification request comprising pharmacy information for a pharmacy; (b) generating, by the one or more processors, a pharmacy enrollment status, comprising (i) accessing the drug distribution management database, (ii) comparing the pharmacy information in the pharmacy enrollment verification request to enrolled pharmacy information in the drug distribution management database, and (iii) obtaining a positive pharmacy enrollment status by identifying a match between the pharmacy information in the pharmacy enrollment verification request and the pharmacy information in the drug distribution management database, or obtaining a negative pharmacy enrollment status if no match between the pharmacy information in the pharmacy enrollment verification request and the pharmacy information in the drug distribution management database is identified; and (c) sending, to a second electronic device associated with the distributor, the pharmacy enrollment status, wherein receipt of a positive pharmacy enrollment status authorizes the distributor to provide the drug to the pharmacy. In some embodiments, the one or more programs further including instructions for: receiving, by the one or more processors, a pharmacy enrollment request comprising pharmacy information for the pharmacy; and adding, by the one or more processors, an entry comprising the pharmacy information into the drug distribution management database to enroll the pharmacy in the drug distribution management database. In some embodiments, the one or more programs further including instructions for: receiving, by the one or more processors, a distributor enrollment request comprising distributor information for the distributor; and adding, by the one or more processors, an entry comprising the distributor information into the drug distribution management database to enroll the distributor in the drug distribution management database.

In some embodiments, a non-transitory computer-readable storage medium storing one or more programs comprising instructions, which when executed by one or more processors of an electronic device, cause the electronic device to: (a) receive, by the one or more processors, a pharmacy enrollment verification request comprising pharmacy information for a pharmacy; (b) generate by the one or more processors, a pharmacy enrollment status by (i) accessing a drug distribution management database for a drug comprising a food allergen, (ii) comparing the pharmacy information in the pharmacy enrollment verification request to enrolled pharmacy information in the drug distribution management database, and (iii) obtaining a positive pharmacy enrollment status by identifying a match between the pharmacy information in the pharmacy enrollment verification request and the pharmacy information in the drug distribution management database, or obtaining a negative pharmacy enrollment status if no match between the pharmacy information in the pharmacy enrollment verification request and the pharmacy information in the drug distribution management database is identified; and (c) send, to a second electronic device associated with the distributor, the pharmacy enrollment status, wherein receipt of a positive pharmacy enrollment status authorizes the distributor to provide the drug to the pharmacy. In some embodiments, the instructions, which when executed by the one or more processors of the electronic device, further cause the electronic device to: receive, by the one or more processors, a pharmacy enrollment request comprising pharmacy information for the pharmacy; and add, by the one or more processors, an entry comprising the pharmacy information into the drug distribution management database to enroll the pharmacy in the drug distribution management database. In some embodiments, the instructions, which when executed by the one or more processors of the electronic device, further cause the electronic device to: receive, by the one or more processors, a distributor enrollment request comprising distributor information for the distributor; and add, by the one or more processors, an entry comprising the distributor information into the drug distribution management database to enroll the distributor in the drug distribution management database.

In some embodiments, a computer-implemented method for authorizing the distribution of an oral immunotherapy treatment kit comprising a plurality of doses of a drug comprising a food allergen at different dosing levels to a healthcare setting, comprises (a) receiving, at a first electronic device communicatively coupled to a drug distribution management database for the drug and from a second electronic device associated with a distributor enrolled in the drug distribution management database, a healthcare setting enrollment verification request comprising healthcare setting information for a healthcare setting; (b) generating, at the first electronic device, a healthcare setting enrollment status, comprising (i) accessing the drug distribution management database, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; and (c) sending, to the second electronic device, the healthcare setting enrollment status, wherein receipt of a positive healthcare setting enrollment status authorizes the distributor to provide the oral immunotherapy treatment kit to the healthcare setting. In some embodiments, the method further comprises receiving, by the first electronic device, a healthcare setting enrollment request comprising healthcare setting information for the healthcare setting; and adding, at the first electronic device, an entry comprising the healthcare setting information into the drug distribution management database to enroll the healthcare setting in the drug distribution management database. In some embodiments, the method further comprises receiving, by the first electronic device, a distributor enrollment request comprising distributor information for the distributor; and adding, at the first electronic device, an entry comprising the distributor information into the drug distribution management database to enroll the distributor in the drug distribution management database. In some embodiments, a computer program product comprises instructions which, when the program is executed by a computer, causes the computer to carry out the steps of the described method.

In some embodiments, an electronic device comprises one or more processors; a memory configured to store a drug distribution management database for a drug comprising a food allergen, the drug distribution management database comprising entries for enrolled distributors comprising enrolled distributor information, and entries for enrolled healthcare settings comprising enrolled healthcare setting information; and one or more programs stored on the memory configured to be executed by the one or more processors, the one or more programs including instructions for: (a) receiving, by the one or more processors, a healthcare setting enrollment verification request comprising healthcare setting information for a healthcare setting; (b) generating, by the one or more processors, a healthcare setting enrollment status, comprising (i) accessing the drug distribution management database, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; and (c) sending, to a second electronic device associated with the distributor, the healthcare setting enrollment status, wherein receipt of a positive healthcare setting enrollment status authorizes the distributor to provide the oral immunotherapy treatment kit to the healthcare setting. In some embodiments, the one or more programs further including instructions for: receiving, by the one or more processors, a healthcare setting enrollment request comprising healthcare setting information for the healthcare setting; and adding, by the one or more processors, an entry comprising the healthcare setting information into the drug distribution management database to enroll the healthcare setting in the drug distribution management database. In some embodiments, the one or more programs further including instructions for: receiving, by the one or more processors, a distributor enrollment request comprising distributor information for the distributor; and adding, by the one or more processors, an entry comprising the distributor information into the drug distribution management database to enroll the distributor in the drug distribution management database.

In some embodiments, a non-transitory computer-readable storage medium storing one or more programs comprising instructions, which when executed by one or more processors of an electronic device, cause the electronic device to: (a) receive, by the one or more processors, a healthcare setting enrollment verification request comprising healthcare setting information for a healthcare setting; (b) generate, by the one or more processors, a healthcare setting enrollment status by (i) accessing a drug distribution management database for a drug comprising a food allergen, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; and (c) send, to a second electronic device associated with the distributor, the healthcare setting enrollment status, wherein receipt of a positive healthcare setting enrollment status authorizes the distributor to provide the oral immunotherapy treatment kit to the healthcare setting. In some embodiments, the instructions, which when executed by the one or more processors of the electronic device, further cause the electronic device to: receive, by the one or more processors, a healthcare setting enrollment request comprising healthcare setting information for the healthcare setting; and add, by the one or more processors, an entry comprising the healthcare setting information into the drug distribution management database to enroll the healthcare setting in the drug distribution management database. In some embodiments, the instructions, which when executed by the one or more processors of the electronic device, further cause the electronic device to: receive, by the one or more processors, a distributor enrollment request comprising distributor information for the distributor; and add, by the one or more processors, an entry comprising the distributor information into the drug distribution management database to enroll the distributor in the drug distribution management database.

In some embodiments, a computer-implemented method for managing the distribution of a drug comprising a food allergen, comprises: (a) authorizing the provision of an initial dose escalation kit comprising plurality of different doses of the drug according to an initial dose escalation schedule, comprising: (i) receiving, at a first electronic device communicatively coupled to a drug distribution management database for the drug and from a second electronic device associated with a pharmacy enrolled in the drug distribution management database, a first healthcare setting enrollment verification request comprising healthcare setting information for a healthcare setting and a first patient enrollment verification request comprising patient information for a patient; (ii) generating, at the first electronic device, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (iii) generating, at the first electronic device, a first patient enrollment status, comprising (1) accessing the drug distribution management database, (2) comparing the patient information in the first patient enrollment verification request to enrolled patient information in the drug distribution management database, and (3) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (iv) sending, to the second electronic device, the first healthcare setting enrollment status and the first patient enrollment status, wherein receipt of a positive first healthcare setting enrollment status and a positive first patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule; (b) authorizing the provision of a plurality of doses of the drug at a single treatment dose level according to an oral immunotherapy dosing schedule, comprising: (i) receiving, at the first electronic device from the second electronic device, a second healthcare setting enrollment verification request comprising healthcare setting information for a healthcare setting and a second patient enrollment verification request comprising patient information for a patient; (ii) generating, at the first electronic device, a second healthcare setting enrollment status, comprising (1) accessing the drug distribution management database, (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; (iii) generating, at the first electronic device, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare setting enrollment status and the second patient enrollment status, wherein receipt of a positive second healthcare setting enrollment status and a positive second patient enrollment status by the pharmacy authorizes the pharmacy to provide to the healthcare setting or the patient a plurality of doses of the drug at a single treatment dose level according to an oral immunotherapy dosing schedule; (c) repeating step (b) at a different treatment dose level, wherein the pharmacy is not authorized to simultaneously provide the drug at more than one treatment dose level for the patient; (d) authorizing the provision of an oral immunotherapy treatment kit comprising a plurality of doses of a drug for treating a food allergy at different up-dosing levels to a healthcare setting, comprising: (i) receiving, at the first electronic device and from a third electronic device associated with a distributor enrolled in the drug distribution management database, a third healthcare setting enrollment verification request comprising healthcare setting information for a healthcare setting; (ii) generating, at the first electronic device, a third healthcare setting enrollment status, comprising (1) accessing the drug distribution management database, and (2) comparing the healthcare setting information in the third healthcare setting enrollment verification request to enrolled healthcare setting information in the drug distribution management database, and (3) obtaining a positive healthcare setting enrollment status by identifying a match between the healthcare setting information in the third healthcare setting enrollment verification request and the healthcare setting information in the drug distribution management database, or obtaining a negative healthcare setting enrollment status if no match between the healthcare setting information in the third healthcare setting enrollment verification request and the healthcare setting information in the drug distribution management database is identified; and (iii) sending, to the third electronic device, the third healthcare setting enrollment status, wherein receipt of a positive third healthcare setting enrollment status authorizes the distributor to provide the oral immunotherapy treatment kit to the healthcare setting. In some embodiments, the method further comprises receiving, by the first electronic device, a patient enrollment request comprising patient information for the patient; and adding, at the first electronic device, an entry comprising the patient information into the drug distribution management database to enroll the patient in the drug distribution management database. In some embodiments, the method further comprises receiving, by the first electronic device, a pharmacy enrollment request comprising pharmacy information for the pharmacy; and adding, at the first electronic device, an entry comprising the pharmacy information into the drug distribution management database to enroll the pharmacy in the drug distribution management database. In some embodiments, the method further comprises receiving, by the first electronic device, a healthcare setting enrollment request comprising healthcare setting information for the healthcare setting; and adding, at the first electronic device, an entry comprising the healthcare setting information into the drug distribution management database to enroll the healthcare setting in the drug distribution management database. In some embodiments, the method further comprises receiving, by the first electronic device, a distributor enrollment request comprising distributor information for the distributor; and adding, at the first electronic device, an entry comprising the distributor information into the drug distribution management database to enroll the distributor in the drug distribution management database. In some embodiments, a computer program product comprising instructions which, when the program is executed by a computer, causes the computer to carry out the steps of the described method.

In some embodiments, a computer-implemented method for managing the distribution of a drug comprising a food allergen, comprises: (a) authorizing the provision of an initial dose escalation kit comprising plurality of different doses of the drug according to an initial dose escalation schedule, comprising: (i) receiving, at a first electronic device communicatively coupled to a drug distribution management database for the drug and from a second electronic device associated with a pharmacy enrolled in the drug distribution management database, a first healthcare setting enrollment verification request comprising healthcare setting information for a healthcare setting, a first healthcare provider enrollment verification request comprising healthcare provider information, and a first patient enrollment verification request comprising patient information for a patient; (ii) generating, at the first electronic device, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (iii) generating, at the first electronic device, a first healthcare provider enrollment status, comprising (1) accessing the drug distribution management database, (2) comparing the healthcare provider information in the first healthcare provider enrollment verification request to enrolled healthcare provider information in the drug distribution management database, and (3) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; (iv) generating, at the first electronic device, a first patient enrollment status, comprising (1) accessing the drug distribution management database, (2) comparing the patient information in the first patient enrollment verification request to enrolled patient information in the drug distribution management database, and (3) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (v) sending, to the second electronic device, the first healthcare setting enrollment status, the first healthcare provider enrollment status, and the first patient enrollment status, wherein receipt of a positive first healthcare setting enrollment status, a positive healthcare provider enrollment status, and a positive first patient enrollment status by the pharmacy authorizes the pharmacy to provide to the healthcare setting or the healthcare provider an initial dose escalation kit associated with the patient, the initial dose escalation kit comprising a plurality of different doses of the drug according to an initial dose escalation schedule; (b) authorizing the provision of a plurality of doses of the drug at a single treatment dose level according to an oral immunotherapy dosing schedule, comprising: (i) receiving, at the first electronic device from the second electronic device, a second healthcare provider enrollment verification request comprising healthcare provider information for a healthcare provider and a second patient enrollment verification request comprising patient information for a patient; (ii) generating, at the first electronic device, a second healthcare provider enrollment status, comprising (1) accessing the drug distribution management database, (2) comparing the healthcare provider information in the second healthcare provider enrollment verification request to enrolled healthcare provider information in the drug distribution management database, and (3) obtaining a positive healthcare provider enrollment status by identifying a match between the healthcare provider information in the second healthcare provider enrollment verification request and the healthcare provider information in the drug distribution management database, or obtaining a negative healthcare provider enrollment status if no match between the healthcare provider information in the second healthcare provider enrollment verification request and the healthcare provider information in the drug distribution management database is identified; (iii) generating, at the first electronic device, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare provider enrollment status and the second patient enrollment status, wherein receipt of a positive second healthcare provider enrollment status and a positive second patient enrollment status by the pharmacy authorizes the pharmacy to provide to the healthcare setting, the healthcare provider, or the patient a plurality of doses of the drug at a single treatment dose level according to an oral immunotherapy dosing schedule; (c) repeating step (b) at a different treatment dose level, wherein the pharmacy is not authorized to simultaneously provide the drug at more than one treatment dose level for the patient; (d) authorizing the provision of an oral immunotherapy treatment kit comprising a plurality of doses of a drug for treating a food allergy at different up-dosing levels to a healthcare setting, comprising: (i) receiving, at the first electronic device and from a third electronic device associated with a distributor enrolled in the drug distribution management database, a second healthcare setting enrollment verification request comprising healthcare setting information for a healthcare setting; (ii) generating, at the first electronic device, a second healthcare setting enrollment status, comprising (1) accessing the drug distribution management database, and (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; and (iii) sending, to the third electronic device, the second healthcare setting enrollment status, wherein receipt of a positive third healthcare setting enrollment status authorizes the distributor to provide the oral immunotherapy treatment kit to the healthcare setting. In some embodiments, the method further comprises: receiving, by the first electronic device, a healthcare provider enrollment request comprising healthcare provider information for the patient; and adding, at the first electronic device, an entry comprising the healthcare provider information into the drug distribution management database to enroll the healthcare provider in the drug distribution management database. In some embodiments, the method further comprises receiving, by the first electronic device, a patient enrollment request comprising patient information for the patient; and adding, at the first electronic device, an entry comprising the patient information into the drug distribution management database to enroll the patient in the drug distribution management database. In some embodiments, the method further comprises receiving, by the first electronic device, a pharmacy enrollment request comprising pharmacy information for the pharmacy; and adding, at the first electronic device, an entry comprising the pharmacy information into the drug distribution management database to enroll the pharmacy in the drug distribution management database. In some embodiments, the method further comprises receiving, by the first electronic device, a healthcare setting enrollment request comprising healthcare setting information for the healthcare setting; and adding, at the first electronic device, an entry comprising the healthcare setting information into the drug distribution management database to enroll the healthcare setting in the drug distribution management database. In some embodiments, the method further comprises receiving, by the first electronic device, a distributor enrollment request comprising distributor information for the distributor; and adding, at the first electronic device, an entry comprising the distributor information into the drug distribution management database to enroll the distributor in the drug distribution management database. In some embodiments, a computer program product comprising instructions which, when the program is executed by a computer, causes the computer to carry out the steps of the described method.

In some embodiments, an electronic device comprises: one or more processors; a memory configured to store a drug distribution management database for a drug comprising a food allergen, the drug distribution management database comprising entries for enrolled patients comprising enrolled patient information, entries for enrolled healthcare settings comprising enrolled healthcare setting information, entries for enrolled pharmacies comprising enrolled pharmacy information, and entries for enrolled distributors comprising enrolled distributor information; and one or more programs stored on the memory configured to be executed by the one or more processors, the one or more programs including instructions for: (a) authorizing, by the one or more processors, the provision of an initial dose escalation kit comprising plurality of different doses of the drug according to an initial dose escalation schedule, comprising: (i) receiving, by the one or more processors and from a second electronic device associated with a pharmacy enrolled in the drug distribution management database, a first healthcare setting enrollment verification request comprising healthcare setting information for a healthcare setting and a first patient enrollment verification request comprising patient information for a patient; (ii) generating, by the one or more processors, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the first healthcare setting enrollment verification request and the healthcare setting information in the drug distribution management database, or obtaining a negative healthcare setting enrollment status if no match between the healthcare setting information in the first healthcare setting enrollment verification request and the healthcare setting information in the drug distribution management database is identified; (iii) generating, by the one or more processors, a first patient enrollment status, comprising (1) accessing the drug distribution management database, and (2) comparing the patient information in the first patient enrollment verification request to enrolled patient information in the drug distribution management database, and (3) obtaining a positive patient enrollment status by identifying a match between the patient information in the first patient enrollment verification request and the patient information in the drug distribution management database, or obtaining a negative patient enrollment status if no match between the patient information in the first patient enrollment verification request and the patient information in the drug distribution management database is identified; and (iv) sending, to the second electronic device, the first healthcare setting enrollment status and the first patient enrollment status, wherein receipt of a positive first healthcare setting enrollment status and a positive first patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule; (b) authorizing, by the one or more processors, a plurality of doses of the drug at a single treatment dose level according to an oral immunotherapy dosing schedule, comprising: (i) receiving, by the one or more processors and from the second electronic device, a second healthcare setting enrollment verification request comprising healthcare setting information for a healthcare setting and a second patient enrollment verification request comprising patient information for a patient; (ii) generating, by the one or more processors, a second healthcare setting enrollment status, comprising (1) accessing the drug distribution management database, (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; (iii) generating, by the one or more processors, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare setting enrollment status and the second patient enrollment status, wherein receipt of a positive second healthcare setting enrollment status and a positive second patient enrollment status by the pharmacy authorizes the pharmacy to provide to the healthcare setting or the patient a plurality of doses of the drug at a single treatment dose level according to an oral immunotherapy dosing schedule; (c) repeating step (b) at a different treatment dose level, wherein the pharmacy is not authorized to simultaneously provide the drug at more than one treatment dose level for the patient; (d) authorizing, by the one or more processors, the provision of an oral immunotherapy treatment kit comprising a plurality of doses of a drug for treating a food allergy at different up-dosing levels to a healthcare setting, comprising: (i) receiving, by the one or more processors and from a third electronic device associated with a distributor enrolled in the drug distribution management database, a third healthcare setting enrollment verification request comprising healthcare setting information for a healthcare setting; (ii) generating, by the one or more processors, a third healthcare setting enrollment status, comprising (1) accessing the drug distribution management database, (2) comparing the healthcare setting information in the third healthcare setting enrollment verification request to enrolled healthcare setting information in the drug distribution management database, and (3) obtaining a positive healthcare setting enrollment status by identifying a match between the healthcare setting information in the third healthcare setting enrollment verification request and the healthcare setting information in the drug distribution management database, or obtaining a negative healthcare setting enrollment status if no match between the healthcare setting information in the third healthcare setting enrollment verification request and the healthcare setting information in the drug distribution management database is identified; and (iii) sending, to the third electronic device, the third healthcare setting enrollment status, wherein receipt of a positive third healthcare setting enrollment status authorizes the distributor to provide the oral immunotherapy treatment kit to the healthcare setting. In some embodiments, the one or more programs further including instructions for: receiving, by the one or more processors, a pharmacy enrollment request comprising pharmacy information for the pharmacy; and adding, by the one or more processors, an entry comprising the pharmacy information into the drug distribution management database to enroll the pharmacy in the drug distribution management database. In some embodiments, the one or more programs further including instructions for: receiving, by the one or more processors, a distributor enrollment request comprising distributor information for the distributor; and adding, by the one or more processors, an entry comprising the distributor information into the drug distribution management database to enroll the distributor in the drug distribution management database. In some embodiments, the one or more programs further including instructions for: receiving, by the one or more processors, a patient enrollment request comprising patient information for the patient; and adding, by the one or more processors, an entry comprising the patient information into the drug distribution management database to enroll the patient in the drug distribution management database. In some embodiments, the one or more programs further including instructions for: receiving, by the one or more processors, a healthcare setting enrollment request comprising healthcare setting information for the healthcare setting; and adding, by the one or more processors, an entry comprising the healthcare setting information into the drug distribution management database to enroll the healthcare setting in the drug distribution management database.

In some embodiments, an electronic device comprises: one or more processors; a memory configured to store a drug distribution management database for a drug comprising a food allergen, the drug distribution management database comprising entries for enrolled patients comprising enrolled patient information, entries for enrolled healthcare settings comprising enrolled healthcare setting information, entries for enrolled healthcare providers comprising enrolled healthcare provider information, entries for enrolled pharmacies comprising enrolled pharmacy information, and entries for enrolled distributors comprising enrolled distributor information; and one or more programs stored on the memory configured to be executed by the one or more processors, the one or more programs including instructions for: (a) authorizing, by the one or more processors, the provision of an initial dose escalation kit comprising plurality of different doses of the drug according to an initial dose escalation schedule, comprising: (i) receiving, by the one or more processors and from a second electronic device associated with a pharmacy enrolled in the drug distribution management database, a first healthcare setting enrollment verification request comprising healthcare setting information for a healthcare setting, a first healthcare provider enrollment verification request comprising healthcare provider information, and a first patient enrollment verification request comprising patient information for a patient; (ii) generating, by the one or more processors, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the first healthcare setting enrollment verification request and the healthcare setting information in the drug distribution management database, or obtaining a negative healthcare setting enrollment status if no match between the healthcare setting information in the first healthcare setting enrollment verification request and the healthcare setting information in the drug distribution management database is identified; (iii) generating, by the one or more processors, a first patient enrollment status, comprising (1) accessing the drug distribution management database, and (2) comparing the patient information in the first patient enrollment verification request to enrolled patient information in the drug distribution management database, and (3) obtaining a positive patient enrollment status by identifying a match between the patient information in the first patient enrollment verification request and the patient information in the drug distribution management database, or obtaining a negative patient enrollment status if no match between the patient information in the first patient enrollment verification request and the patient information in the drug distribution management database is identified; and (iv) generating, by the one or more processors, a first healthcare provider enrollment status, comprising (1) accessing the drug distribution management database, (2) comparing the healthcare provider information in the first healthcare provider enrollment verification request to enrolled healthcare provider information in the drug distribution management database, and (3) obtaining a positive healthcare provider enrollment status by identifying a match between the healthcare provider information in the first healthcare provider enrollment verification request and the healthcare provider information in the drug distribution management database, or obtaining a negative healthcare provider enrollment status if no match between the healthcare provider information in the first healthcare provider enrollment verification request and the healthcare provider information in the drug distribution management database is identified; (v) sending, to the second electronic device, the first healthcare setting enrollment status, the first healthcare provider enrollment status, and the first patient enrollment status, wherein receipt of a positive first healthcare setting enrollment status, a positive first healthcare provider enrollment status, and a positive first patient enrollment status by the pharmacy authorizes the pharmacy to provide to the healthcare setting or healthcare provider an initial dose escalation kit associated with the patient, the initial dose escalation kit comprising a plurality of different doses of the drug according to an initial dose escalation schedule; (b) authorizing, by the one or more processors, a plurality of doses of the drug at a single treatment dose level according to an oral immunotherapy dosing schedule, comprising: (i) receiving, by the one or more processors and from the second electronic device, a second healthcare provider enrollment verification request comprising healthcare provider information for a healthcare provider and a second patient enrollment verification request comprising patient information for a patient; (ii) generating, by the one or more processors, a second healthcare provider enrollment status, comprising (1) accessing the drug distribution management database, (2) comparing the healthcare provider information in the second healthcare provider enrollment verification request to enrolled healthcare provider information in the drug distribution management database, and (3) obtaining a positive healthcare provider enrollment status by identifying a match between the healthcare setting information in the second healthcare provider enrollment verification request and the healthcare provider information in the drug distribution management database, or obtaining a negative healthcare provider enrollment status if no match between the healthcare provider information in the second healthcare provider enrollment verification request and the healthcare provider information in the drug distribution management database is identified; (iii) generating, by the one or more processors, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare provider enrollment status and the second patient enrollment status, wherein receipt of a positive second healthcare provider enrollment status and a positive second patient enrollment status by the pharmacy authorizes the pharmacy to provide to the healthcare setting, the healthcare provider, or the patient a plurality of doses of the drug at a single treatment dose level according to an oral immunotherapy dosing schedule; (c) repeating step (b) at a different treatment dose level, wherein the pharmacy is not authorized to simultaneously provide the drug at more than one treatment dose level for the patient; (d) authorizing, by the one or more processors, the provision of an oral immunotherapy treatment kit comprising a plurality of doses of a drug for treating a food allergy at different up-dosing levels to a healthcare setting, comprising; (i) receiving, by the one or more processors and from a third electronic device associated with a distributor enrolled in the drug distribution management database, a second healthcare setting enrollment verification request comprising healthcare setting information for a healthcare setting; (ii) generating, by the one or more processors, a second healthcare setting enrollment status, comprising (1) accessing the drug distribution management database, (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; and (iii) sending, to the third electronic device, the second healthcare setting enrollment status, wherein receipt of a positive second healthcare setting enrollment status authorizes the distributor to provide the oral immunotherapy treatment kit to the healthcare setting. In some embodiments, the one or more programs further including instructions for: receiving, by the one or more processors, a healthcare provider enrollment request comprising healthcare provider information for the healthcare provider; and adding, by the one or more processors, an entry comprising the healthcare provider information into the drug distribution management database to enroll the healthcare provider in the drug distribution management database. In some embodiments, the one or more programs further including instructions for: receiving, by the one or more processors, a pharmacy enrollment request comprising pharmacy information for the pharmacy; and adding, by the one or more processors, an entry comprising the pharmacy information into the drug distribution management database to enroll the pharmacy in the drug distribution management database. In some embodiments, the one or more programs further including instructions for: receiving, by the one or more processors, a distributor enrollment request comprising distributor information for the distributor; and adding, by the one or more processors, an entry comprising the distributor information into the drug distribution management database to enroll the distributor in the drug distribution management database. In some embodiments, the one or more programs further including instructions for: receiving, by the one or more processors, a patient enrollment request comprising patient information for the patient; and adding, by the one or more processors, an entry comprising the patient information into the drug distribution management database to enroll the patient in the drug distribution management database. In some embodiments, the one or more programs further including instructions for: receiving, by the one or more processors, a healthcare setting enrollment request comprising healthcare setting information for the healthcare setting; and adding, by the one or more processors, an entry comprising the healthcare setting information into the drug distribution management database to enroll the healthcare setting in the drug distribution management database.

In some embodiments, a non-transitory computer-readable storage medium storing one or more programs comprising instructions, which when executed by one or more processors of an electronic device, cause the electronic device to: (a) authorize, by the one or more processors, the provision of an initial dose escalation kit comprising plurality of different doses of the drug comprising a food allergen according to an initial dose escalation schedule by: (i) receiving, by the one or more processors and from a second electronic device associated with a pharmacy enrolled in the drug distribution management database, a first healthcare setting enrollment verification request comprising healthcare setting information for a healthcare setting and a first patient enrollment verification request comprising patient information for a patient; (ii) generating, by the one or more processors, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the first healthcare setting enrollment verification request and the healthcare setting information in the drug distribution management database, or obtaining a negative healthcare setting enrollment status if no match between the healthcare setting information in the first healthcare setting enrollment verification request and the healthcare setting information in the drug distribution management database is identified; (iii) generating, by the one or more processors, a first patient enrollment status, comprising (1) accessing the drug distribution management database, and (2) comparing the patient information in the first patient enrollment verification request to enrolled patient information in the drug distribution management database, and (3) obtaining a positive patient enrollment status by identifying a match between the patient information in the first patient enrollment verification request and the patient information in the drug distribution management database, or obtaining a negative patient enrollment status if no match between the patient information in the first patient enrollment verification request and the patient information in the drug distribution management database is identified; and (iv) sending, to the second electronic device, the first healthcare setting enrollment status and the first patient enrollment status, wherein receipt of a positive first healthcare setting enrollment status and a positive first patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule; (b) authorize, by the one or more processors, a plurality of doses of the drug at a single treatment dose level according to an oral immunotherapy dosing schedule by: (i) receiving, by the one or more processors and from the second electronic device, a second healthcare setting enrollment verification request comprising healthcare setting information for a healthcare setting and a second patient enrollment verification request comprising patient information for a patient; (ii) generating, by the one or more processors, a second healthcare setting enrollment status, comprising (1) accessing the drug distribution management database, (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; (iii) generating, by the one or more processors, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare setting enrollment status and the second patient enrollment status, wherein receipt of a positive second healthcare setting enrollment status and a positive second patient enrollment status by the pharmacy authorizes the pharmacy to provide to the healthcare setting or the patient a plurality of doses of the drug at a single treatment dose level according to an oral immunotherapy dosing schedule; (c) repeat step (b) at a different treatment dose level, wherein the pharmacy is not authorized to simultaneously provide the drug at more than one treatment dose level for the patient; (d) authorize, by the one or more processors, the provision of an oral immunotherapy treatment kit comprising a plurality of doses of a drug for treating a food allergy at different up-dosing levels to a healthcare setting, by: (i) receiving, by the one or more processors and from a third electronic device associated with a distributor enrolled in the drug distribution management database, a third healthcare setting enrollment verification request comprising healthcare setting information for a healthcare setting; (ii) generating, by the one or more processors, a third healthcare setting enrollment status, comprising (1) accessing the drug distribution management database, (2) comparing the healthcare setting information in the third healthcare setting enrollment verification request to enrolled healthcare setting information in the drug distribution management database, and (3) obtaining a positive healthcare setting enrollment status by identifying a match between the healthcare setting information in the third healthcare setting enrollment verification request and the healthcare setting information in the drug distribution management database, or obtaining a negative healthcare setting enrollment status if no match between the healthcare setting information in the third healthcare setting enrollment verification request and the healthcare setting information in the drug distribution management database is identified; and (iii) sending, to the third electronic device, the third healthcare setting enrollment status, wherein receipt of a positive third healthcare setting enrollment status authorizes the distributor to provide the oral immunotherapy treatment kit to the healthcare setting. In some embodiments, the instructions, which when executed by the one or more processors of the electronic device, further cause the electronic device to: receive, by the one or more processors, a pharmacy enrollment request comprising pharmacy information for the pharmacy; and add, by the one or more processors, an entry comprising the pharmacy information into the drug distribution management database to enroll the pharmacy in the drug distribution management database. In some embodiments, the instructions, which when executed by the one or more processors of the electronic device, further cause the electronic device to: receive, by the one or more processors, a distributor enrollment request comprising distributor information for the distributor; and add, by the one or more processors, an entry comprising the distributor information into the drug distribution management database to enroll the distributor in the drug distribution management database. In some embodiments, the instructions, which when executed by the one or more processors of the electronic device, further cause the electronic device to: receive, by the one or more processors, a patient enrollment request comprising patient information for the patient; and add, by the one or more processors, an entry comprising the patient information into the drug distribution management database to enroll the patient in the drug distribution management database. In some embodiments, the instructions, which when executed by the one or more processors of the electronic device, further cause the electronic device to: receive, by the one or more processors, a healthcare setting enrollment request comprising healthcare setting information for the healthcare setting; and add, by the one or more processors, an entry comprising the healthcare setting information into the drug distribution management database to enroll the healthcare setting in the drug distribution management database.

In some embodiments, a non-transitory computer-readable storage medium storing one or more programs comprising instructions, which when executed by one or more processors of an electronic device, cause the electronic device to: (a) authorize, by the one or more processors, the provision of an initial dose escalation kit comprising plurality of different doses of the drug comprising a food allergen according to an initial dose escalation schedule by; (i) receiving, by the one or more processors and from a second electronic device associated with a pharmacy enrolled in the drug distribution management database, a first healthcare setting enrollment verification request comprising healthcare setting information for a healthcare setting, a first healthcare provider enrollment verification request comprising healthcare provider information, and a first patient enrollment verification request comprising patient information for a patient; (ii) generating, by the one or more processors, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the first healthcare setting enrollment verification request and the healthcare setting information in the drug distribution management database, or obtaining a negative healthcare setting enrollment status if no match between the healthcare setting information in the first healthcare setting enrollment verification request and the healthcare setting information in the drug distribution management database is identified; (iii) generating, by the one or more processors, a first patient enrollment status, comprising (1) accessing the drug distribution management database, and (2) comparing the patient information in the first patient enrollment verification request to enrolled patient information in the drug distribution management database, and (3) obtaining a positive patient enrollment status by identifying a match between the patient information in the first patient enrollment verification request and the patient information in the drug distribution management database, or obtaining a negative patient enrollment status if no match between the patient information in the first patient enrollment verification request and the patient information in the drug distribution management database is identified; (iv) generating, by the one or more processors, a first healthcare provider enrollment status, comprising (1) accessing the drug distribution management database, (2) comparing the healthcare provider information in the first healthcare provider enrollment verification request to enrolled healthcare provider information in the drug distribution management database, and (3) obtaining a positive healthcare provider enrollment status by identifying a match between the healthcare provider information in the first healthcare provider enrollment verification request and the healthcare provider information in the drug distribution management database, or obtaining a negative healthcare provider enrollment status if no match between the healthcare provider information in the first healthcare provider enrollment verification request and the healthcare setting information in the drug distribution management database is identified; (v) sending, to the second electronic device, the first healthcare setting enrollment status, the first healthcare provider enrollment status, and the first patient enrollment status, wherein receipt of a positive first healthcare setting enrollment status, a positive first healthcare provider enrollment status, and a positive first patient enrollment status by the pharmacy authorizes the pharmacy to provide to the healthcare setting or healthcare provider an initial dose escalation kit associated with the patient, the initial dose escalation kit comprising a plurality of different doses of the drug according to an initial dose escalation schedule; (b) authorize, by the one or more processors, a plurality of doses of the drug at a single treatment dose level according to an oral immunotherapy dosing schedule by; (i) receiving, by the one or more processors and from the second electronic device, a second healthcare provider enrollment verification request comprising healthcare provider information for a healthcare provider and a second patient enrollment verification request comprising patient information for a patient; (ii) generating, by the one or more processors, a second healthcare provider enrollment status, comprising (1) accessing the drug distribution management database, (2) comparing the healthcare provider information in the second healthcare provider enrollment verification request to enrolled healthcare provider information in the drug distribution management database, and (3) obtaining a positive healthcare provider enrollment status by identifying a match between the healthcare provider information in the second healthcare provider enrollment verification request and the healthcare provider information in the drug distribution management database, or obtaining a negative healthcare provider enrollment status if no match between the healthcare provider information in the second healthcare provider enrollment verification request and the healthcare provider information in the drug distribution management database is identified; (iii) generating, by the one or more processors, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare provider enrollment status and the second patient enrollment status, wherein receipt of a positive second healthcare provider enrollment status and a positive second patient enrollment status by the pharmacy authorizes the pharmacy to provide to the healthcare setting, the healthcare provider, or the patient a plurality of doses of the drug at a single treatment dose level according to an oral immunotherapy dosing schedule; (c) repeat step (b) at a different treatment dose level, wherein the pharmacy is not authorized to simultaneously provide the drug at more than one treatment dose level for the patient; (d) authorize, by the one or more processors, the provision of an oral immunotherapy treatment kit comprising a plurality of doses of a drug for treating a food allergy at different up-dosing levels to a healthcare setting, by: (i) receiving, by the one or more processors and from a third electronic device associated with a distributor enrolled in the drug distribution management database, a second healthcare setting enrollment verification request comprising healthcare setting information for a healthcare setting; (ii) generating, by the one or more processors, a second healthcare setting enrollment status, comprising (1) accessing the drug distribution management database, (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; and (iii) sending, to the third electronic device, the second healthcare setting enrollment status, wherein receipt of a positive third healthcare setting enrollment status authorizes the distributor to provide the oral immunotherapy treatment kit to the healthcare setting. In some embodiments, the instructions, which when executed by the one or more processors of the electronic device, further cause the electronic device to: receive, by the one or more processors, a healthcare provider enrollment request comprising healthcare provider information for the healthcare provider; and add, by the one or more processors, an entry comprising the healthcare provider information into the drug distribution management database to enroll the healthcare provider in the drug distribution management database. In some embodiments, the instructions, which when executed by the one or more processors of the electronic device, further cause the electronic device to: receive, by the one or more processors, a pharmacy enrollment request comprising pharmacy information for the pharmacy; and add, by the one or more processors, an entry comprising the pharmacy information into the drug distribution management database to enroll the pharmacy in the drug distribution management database. In some embodiments, the instructions, which when executed by the one or more processors of the electronic device, further cause the electronic device to: receive, by the one or more processors, a distributor enrollment request comprising distributor information for the distributor; and add, by the one or more processors, an entry comprising the distributor information into the drug distribution management database to enroll the distributor in the drug distribution management database. In some embodiments, the instructions, which when executed by the one or more processors of the electronic device, further cause the electronic device to: receive, by the one or more processors, a patient enrollment request comprising patient information for the patient; and add, by the one or more processors, an entry comprising the patient information into the drug distribution management database to enroll the patient in the drug distribution management database. In some embodiments, the instructions, which when executed by the one or more processors of the electronic device, further cause the electronic device to: receive, by the one or more processors, a healthcare setting enrollment request comprising healthcare setting information for the healthcare setting; and add, by the one or more processors, an entry comprising the healthcare setting information into the drug distribution management database to enroll the healthcare setting in the drug distribution management database.

In some embodiments, a method of treating a patient for a food allergy by oral immunotherapy, comprises (a) enrolling the patient or verifying enrollment of the patient in a drug distribution management database for a drug comprising a food allergen; (b) administering to the enrolled patient, in a healthcare setting enrolled in the drug distribution management database, a dose of the drug at a first up-dosing level according to an up-dosing schedule; (c) monitoring the enrolled patient, in the enrolled healthcare setting, for an allergic response to the drug after administering the dose of the drug in step (b); (d) providing a first prescription requesting a pharmacy enrolled in the drug distribution management database release to the enrolled patient or the enrolled healthcare setting a plurality of doses of the drug at the first up-dosing level; (e) administering to the enrolled patient, in the enrolled healthcare setting, a dose of the drug at a subsequent up-dosing level according to the up-dosing schedule; (f) monitoring the enrolled patient, in the enrolled healthcare setting, for an allergic response to the drug after administering the dose of the drug in step (e); and (g) providing a subsequent prescription requesting the enrolled pharmacy release to the enrolled patient or the enrolled healthcare setting a plurality of doses of the drug at the subsequent up-dosing level. In some embodiments, the dose of (b) or (e) is administered under the supervision of a healthcare provider enrolled in the drug distribution management database. In some embodiments, the monitoring of (c) or (f) is under the supervision of a healthcare provider enrolled in the drug distribution management database. In some embodiments, the method comprises obtaining the dose of the drug administered to the enrolled patient in step (b) or step (e) from an oral immunotherapy treatment kit comprising a plurality of doses of the drug at different up-dosing levels. In some embodiments, the oral immunotherapy treatment kit is received from a distributor enrolled in the drug distribution management database. In some embodiments, the method comprises submitting a request to the enrolled distributor to provide the oral immunotherapy treatment kit to the enrolled healthcare setting, wherein providing the oral immunotherapy treatment kit to the enrolled healthcare setting is conditioned on the enrolled distributor verifying enrollment of the healthcare setting. In some embodiments, the method comprises, prior to step (b), administering to the enrolled patient, in the enrolled healthcare setting, a plurality of different doses of the drug according to an initial dose escalation schedule. In some embodiments, the plurality of different doses of the drug according to the initial dose escalation schedule are administered under the supervision of a healthcare provider enrolled in the drug distribution management database. In some embodiments, the method comprises monitoring the enrolled patient, in the enrolled healthcare setting, for an allergic response to the drug after administering each different dose of the drug according to the initial dose escalation schedule. In some embodiments, monitoring after administering each different dose of the drug according to the initial dose escalation schedule is under the supervision of a healthcare provider enrolled in the drug distribution management database. In some embodiments, the plurality of different doses of the drug according to the initial dose escalation schedule is obtained from an initial dose escalation kit assigned to the enrolled patient. In some embodiments, the method comprises receiving the initial dose escalation kit from the enrolled pharmacy. In some embodiments, the method comprises submitting a request to the enrolled pharmacy to provide the initial dose escalation kit to the enrolled healthcare setting, wherein providing the initial dose escalation kit to the enrolled healthcare setting is conditioned on the enrolled pharmacy verifying (1) enrollment of the patient in the drug distribution management database and (2) enrollment of the healthcare setting. In some embodiments, providing the initial dose escalation kit to the enrolled healthcare setting is further conditioned on the enrolled pharmacy verifying enrollment of a healthcare provider in the drug distribution management database, wherein the healthcare provider is associated with the enrolled patient. In some embodiments, the invention provides a drug comprising a food allergen for use in treating a patient for a food allergy by oral immunotherapy, wherein the drug is administered to the patient according to a method as described above.

In some embodiments, method of initiating treatment of a patient for a food allergy by oral immunotherapy, comprises: (a) enrolling the patient or verifying enrollment of the patient in a drug distribution management database for a drug comprising a food allergen; (b) administering to the enrolled patient, in the enrolled healthcare setting, a plurality of different doses of the drug according to an initial dose escalation schedule; and (c) monitoring the enrolled patient, in the enrolled healthcare setting, for an allergic response to the drug after administering each different dose of the drug according to the initial dose escalation schedule in step (b). In some embodiments, the plurality of different doses of step (b) are administered under the supervision of a healthcare provider enrolled in the drug distribution management database. In some embodiments, the monitoring of step (c) is under the supervision of a healthcare provider enrolled in the drug distribution management database. In some embodiments, the plurality of different doses of the drug according to the initial dose escalation schedule are obtained from an initial dose escalation kit assigned to the enrolled patient. In some embodiments, the method comprises receiving the initial dose escalation kit from a pharmacy enrolled in the drug distribution management database. In some embodiments, the method comprises submitting a request to the enrolled pharmacy to provide the initial dose escalation kit to the enrolled healthcare setting, wherein providing the initial dose escalation kit to the enrolled healthcare setting is conditioned on the enrolled pharmacy verifying (1) enrollment of the patient in the drug distribution management database and (2) enrollment of the healthcare setting. In some embodiments, providing the initial dose escalation kit to the enrolled healthcare setting is further conditioned on the enrolled pharmacy verifying enrollment of a healthcare provider in the drug distribution management database, wherein the healthcare provider is associated with the enrolled patient. In some embodiments, the invention provides a drug comprising a food allergen for use in treating a patient for a food allergy by oral immunotherapy, wherein the drug is administered to the patient according to a method as described above.

In some embodiments, a method of continuing treatment of a patient for a food allergy by oral immunotherapy, comprising (a) enrolling the patient or verifying enrollment of the patient in a drug distribution management database for a drug comprising a food allergen; (b) administering to the patient, in the enrolled healthcare setting and after enrolling the patient or verifying enrollment of the patient in step (a), a dose of the drug at a dose level higher than any than any previous dose level administered to the patient; and (c) monitoring the patient, in the enrolled healthcare setting, for an allergic response to the drug after administering the drug in step (b). In some embodiments, the dose of step (b) is administered under the supervision of a healthcare provider enrolled in the drug distribution management database. In some embodiments, the monitoring of step (c) is under the supervision of a healthcare provider enrolled in the drug distribution management database. In some embodiments, the dose of the drug is obtained from an oral immunotherapy dosing kit comprising a plurality of doses of the drug at different dose levels. In some embodiments, doses of the drug obtained from the oral immunotherapy dosing kit are administered only to patients enrolled in the drug distribution management database. In some embodiments, doses of the drug obtained from the oral immunotherapy dosing kit are not used to initiate treatment of the patient. In some embodiments, the oral immunotherapy treatment kit is received from a distributor enrolled in the drug distribution management database. In some embodiments, the method comprises submitting a request to the enrolled distributor to provide the oral immunotherapy treatment kit to the enrolled healthcare setting, wherein providing the oral immunotherapy treatment kit to the enrolled healthcare setting is conditioned on the enrolled distributor verifying enrollment of the healthcare setting. In some embodiments, the invention provides a drug comprising a food allergen for use in treating a patient for a food allergy by oral immunotherapy, wherein the drug is administered to the patient according to a method as described above.

In some embodiments of any one of the treatment methods described above, the method comprises submitting an enrollment request for the healthcare setting, and receiving an enrollment verification for the healthcare setting. In some embodiments, the enrollment request affirms that the healthcare setting is equipped to identify and treat a systemic allergic reaction. In some embodiments, the enrollment request affirms that the healthcare setting will monitor the patient for an allergic response to the drug after administering the drug to the patient. In some embodiments, the enrollment request affirms that the healthcare setting will, for every patient to be treated with the drug by the healthcare setting, enroll the patient in or verify enrollment of the patient in the drug distribution management database. In some embodiments, the enrollment request affirms that the healthcare setting will only use an initial dose escalation kit for initiating treatment of a patient uniquely associated with the initial dose escalation kit.

In some embodiments of any one of the treatment methods described above, the method comprises generating a record for each dose of the drug administered to the enrolled patient in the enrolled healthcare setting, wherein the record identifies the administration of the drug and the monitoring of the enrolled patient following administration of the drug.

In some embodiments of any one of the treatment methods described above, the method comprises recommending to the patient that the patient have an anaphylaxis-response treatment immediately available to the patient during the course of treatment. In some embodiments, the anaphylaxis response treatment comprises epinephrine. In some embodiments, the method comprises generating a record confirming that the recommendation that the patient have an anaphylaxis-response treatment immediately available to the patient during the course of treatment has been made to the patient.

In some embodiments of any one of the treatment methods described above, the method comprises recommending to the patient that the patient avoid dietary consumption of the food during the course of treatment. In some embodiments, the method comprises generating a record confirming that the recommendation that the patient avoid dietary consumption of the food during the course of treatment has been made to the patient.

In some embodiments of any one of the treatment methods described above, the method comprises providing a patient status to a drug distribution management database manager upon discontinuation of treatment by the patient or transfer of care of the patient to a subsequent healthcare setting.

In some embodiments, the invention provides a drug comprising a food allergen for use in treating a patient for a food allergy by oral immunotherapy, wherein the drug is administered to the patient according to a method as described above.

In some embodiments, a method of providing a drug comprising a food allergen to a healthcare setting or a patient, comprises (a) receiving, at a pharmacy enrolled in a drug distribution management database for the drug, a request to provide to a healthcare setting an initial dose escalation kit associated with an identified patient, the initial dose escalation kit comprising a plurality of different doses of the drug according to an initial dose escalation schedule; (b) verifying enrollment of the identified patient in the drug distribution management database; (c) verifying enrollment of the healthcare setting in the drug distribution management database; (d) providing, to the healthcare setting, the initial dose escalation kit associated with the identified patient; (e) receiving, at the enrolled pharmacy, a prescription issued by the healthcare setting to provide a plurality of doses of the drug at an up-dosing level according to an up-dosing schedule or a plurality of doses of the drug at a maintenance level; (f) repeating steps (b) and (c) after step (e); and (g) providing, to the patient or the healthcare setting, the plurality of doses of the drug at the up-dosing level or the plurality of doses of the drug at the maintenance level.

In some embodiments, a method of providing a drug comprising a food allergen to a healthcare setting, comprises (a) receiving, at a pharmacy enrolled in a drug distribution management database for the drug, a request to provide to a healthcare setting an initial dose escalation kit associated with an identified patient, the initial dose escalation kit comprising a plurality of different doses of the drug according to an initial dose escalation schedule; (b) verifying enrollment of the identified patient in a drug distribution management database for the drug; (c) verifying enrollment of the healthcare setting in the drug distribution management database; and (d) providing, to the healthcare setting, the initial dose escalation kit associated with the identified patient.

In some embodiments, a method of providing a drug comprising a food allergen to a patient or healthcare setting, comprises (a) receiving, at a pharmacy enrolled in a drug distribution management database for the drug, a prescription issued by the healthcare setting to provide a plurality of doses of the drug at an up-dosing level according to an up-dosing schedule or a plurality of doses of the drug at a maintenance level; (b) verifying enrollment of the identified patient in a drug distribution management database for the drug; (c) verifying enrollment of the healthcare setting in the drug distribution management database; and (d) providing, to the patient or the healthcare setting, the plurality of doses of the drug at the up-dosing level or the plurality of doses of the drug at the maintenance level.

In some embodiments of the method of providing the drug described above, the method comprises submitting an enrollment request for the pharmacy, and receiving an enrollment verification for the pharmacy.

In some embodiments of the method of providing the drug described above, the method comprises generating a record for the patient that associates a treatment level of the provided plurality of doses with the identified patient, wherein the treatment level indicates an initial dose escalation level, the up-dosing level, or a maintenance dose level.

In some embodiments, a method of providing an oral immunotherapy treatment kit comprising a plurality of doses of a drug comprising a food allergen at different levels, comprises (a) receiving, at a distributor enrolled in a drug distribution management database for the drug, a request to provide the oral immunotherapy treatment kit to a healthcare setting; (b) verifying enrollment of the healthcare setting in the drug distribution management database for the drug; and (c) providing the oral immunotherapy treatment kit to the healthcare setting only if the healthcare setting is enrolled in the drug distribution management database. In some embodiments, the method comprises submitting an enrollment request for the distributor, and receiving an enrollment verification for the distributor. In some embodiments, the method comprises generating a record identifying the healthcare setting that has been provided the oral immunotherapy treatment kit. In some embodiments, the request to provide the oral immunotherapy treatment kit to the healthcare setting is made by the healthcare setting.

In some embodiments, a method of treating a patient for a food allergy by oral immunotherapy comprises: (a) enrolling the patient or verifying enrollment of the patient in a drug distribution management database for a drug comprising a food allergen; (b) administering to the enrolled patient a dose of the drug at a first up-dosing level according to an up-dosing schedule, wherein the dose is administered under the supervision of a healthcare provider enrolled in the drug distribution management database; (c) monitoring the enrolled patient, under the supervision of the enrolled healthcare provider, for an allergic response to the drug after administering the dose of the drug in step (b); (d) providing a first prescription requesting a pharmacy enrolled in the drug distribution management database release to the enrolled patient or the enrolled healthcare provider a plurality of doses of the drug at the first up-dosing level; (e) administering to the enrolled patient a dose of the drug at a subsequent up-dosing level according to the up-dosing schedule, wherein the dose is administered under the supervision of the healthcare provider; (f) monitoring the enrolled patient for an allergic response to the drug after administering the dose of the drug in step (e); and (g) providing a subsequent prescription requesting the enrolled pharmacy release to the enrolled patient, the enrolled healthcare provider, or a healthcare setting enrolled in the drug distribution management database a plurality of doses of the drug at the subsequent up-dosing level. In some embodiments, the method further comprises counseling the enrolled patient, wherein counseling is performed under the supervision of the enrolled healthcare provider. In some embodiments, the drug administered to the enrolled patient in step (b) or step (e) is administered in a healthcare setting enrolled in the drug distribution management database. In some embodiments, the method further comprises obtaining the dose of the drug administered to the enrolled patient in step (b) or step (e) from an oral immunotherapy treatment kit comprising a plurality of doses of the drug at different up-dosing levels. In some embodiments, the oral immunotherapy treatment kit is received from a distributor enrolled in the drug distribution management database. In some embodiments, the method further comprises submitting a request to the enrolled distributor to provide the oral immunotherapy treatment kit to the enrolled healthcare provider or enrolled healthcare setting, wherein providing the oral immunotherapy treatment kit to the enrolled healthcare provider or enrolled healthcare setting is conditioned on the enrolled distributor verifying enrollment of the healthcare provider if the oral immunotherapy treatment kit is to be provided to the healthcare provider, or verifying enrollment of the healthcare setting if the oral immunotherapy treatment kit is to be provided to the healthcare setting. In some embodiments, the method further comprises, prior to step (b), administering to the enrolled patient a plurality of different doses of the drug according to an initial dose escalation schedule, wherein the administration is under the supervision of the enrolled healthcare provider. In some embodiments, the method further comprises monitoring the enrolled patient for an allergic response to the drug after administering each different dose of the drug according to the initial dose escalation schedule. In some embodiments, the plurality of different doses of the drug according to the initial dose escalation schedule is obtained from an initial dose escalation kit assigned to the enrolled patient. In some embodiments, the method further comprises receiving the initial dose escalation kit from the enrolled pharmacy. In some embodiments, the method further comprises submitting a request to the enrolled pharmacy to provide the initial dose escalation kit to the enrolled healthcare provider, wherein providing the initial dose escalation kit to the enrolled healthcare provider is conditioned on the enrolled pharmacy verifying (1) enrollment of the patient in the drug distribution management database and (2) enrollment of the healthcare provider. In some embodiments, providing the initial dose escalation kit to the enrolled healthcare provider is further conditioned on the enrolled pharmacy verifying enrollment of a healthcare setting in the drug distribution management database, wherein the initial dose escalation kit is to be administered to the enrolled patient in the enrolled healthcare setting. In some embodiments, the invention provides a drug comprising a food allergen for use in treating a patient for a food allergy by oral immunotherapy, wherein the drug is administered to the patient according to a method as described above.

In some embodiments, there is a method of initiating treatment of a patient for a food allergy by oral immunotherapy comprising: (a) enrolling the patient or verifying enrollment of the patient in a drug distribution management database for a drug comprising a food allergen; (b) administering to the enrolled patient a plurality of different doses of the drug according to an initial dose escalation schedule, wherein the administration is under the supervision of a healthcare provider enrolled in the drug distribution management database; and (c) monitoring the enrolled patient for an allergic response to the drug after administering each different dose of the drug according to the initial dose escalation schedule in step (b). In some embodiments, the administrations of step (b) are in a healthcare setting enrolled in the drug distribution management database. In some embodiments, the monitoring of step (c) is in a healthcare setting enrolled in the drug distribution management database. In some embodiments, the plurality of different doses of the drug according to the initial dose escalation schedule are obtained from an initial dose escalation kit assigned to the enrolled patient. In some embodiments, the method further comprises receiving the initial dose escalation kit from a pharmacy enrolled in the drug distribution management database. In some embodiments, the method further comprises submitting a request to the enrolled pharmacy to provide the initial dose escalation kit to the enrolled healthcare provider or a healthcare setting enrolled in the drug distribution management database, wherein providing the initial dose escalation kit is conditioned on verifying enrollment of the patient in the drug distribution management database and: i) verifying enrollment of the healthcare provider in the drug distribution management database if the initial dose escalation kit is to be provided to the healthcare provider, or ii) verifying enrollment of the healthcare setting in the drug distribution management database if the initial dose escalation kit is to be provided to the healthcare setting. In some embodiments, the invention provides a drug comprising a food allergen for use in treating a patient for a food allergy by oral immunotherapy, wherein the drug is administered to the patient according to a method as described above.

In some embodiments, a method for authorizing the provision of a drug comprising a food allergen to a healthcare setting comprises: (a) verifying enrollment of the healthcare setting in a drug distribution management database; (b) verifying enrollment of a patient in the drug distribution management database; (c) authorizing, after enrollment of the healthcare setting and the patient in the drug distribution management database has been verified, a pharmacy to provide to the healthcare setting an initial dose escalation kit associated with the patient, the initial dose escalation kit comprising a plurality of different doses of the drug according to an initial dose escalation schedule. In some embodiments, the method further comprises verifying enrollment of a healthcare provider in the drug distribution management database, wherein step (c) occurs after enrollment of the healthcare provider in the drug distribution management database has been verified.

In some embodiments, a method for authorizing the provision of a drug comprising a food allergen to a healthcare setting comprises: (a) receiving a healthcare setting enrollment verification request and a patient enrollment verification request; (b) generating a healthcare setting enrollment status indicating whether the healthcare setting associated with the healthcare setting enrollment verification request is enrolled in a drug distribution management database; (c) generating a patient enrollment status indicating whether a patient associated with the patient enrollment verification request is enrolled in the drug distribution management database; and (d) sending the healthcare setting enrollment status and the patient enrollment status to a pharmacy enrolled in the drug distribution management database to authorize the pharmacy to provide to the healthcare setting an initial dose escalation kit associated with the patient, the initial dose escalation kit comprising a plurality of different doses of the drug according to an initial dose escalation schedule. In some embodiments, the method further comprises: receiving a healthcare provider enrollment verification request; generating a healthcare provider enrollment status indicating whether a healthcare provider associated with the healthcare provider enrollment verification request is enrolled in the drug distribution management database; and sending the healthcare provider enrollment status to the pharmacy enrolled in the drug distribution management database to authorize the pharmacy to provide to the healthcare setting an initial dose escalation kit associated with the patient.

In some embodiments, a method of authorizing the provisional of a drug comprising a food allergen to a healthcare setting or a patient comprises: (a) verifying enrollment of a healthcare provider in a drug distribution management database; (b) verifying enrollment of a patient in the drug distribution management database; (c) authorizing, after enrollment of the healthcare provider and the patient in the drug distribution management database has been verified, a pharmacy to provide to the healthcare setting or the patient a plurality of doses of the drug at a single treatment dose level according to an oral immunotherapy dosing schedule. In some embodiments, the method further comprises verifying enrollment of a healthcare setting in a drug distribution management database, wherein step (c) occurs after enrollment of the healthcare setting in the drug distribution management database has been verified.

In some embodiments, a method for authorizing the provision of a drug comprising a food allergen to a healthcare setting or a patient comprises: (a) receiving a healthcare provider enrollment verification request and a patient enrollment verification request; (b) generating a healthcare provider enrollment status indicating whether a healthcare provider associated with the healthcare setting enrollment verification request is enrolled in a drug distribution management database; (c) generating a patient enrollment status indicating whether the patient associated with the patient enrollment verification request is enrolled in the drug distribution management database; and (d) sending the healthcare provider enrollment status and the patient enrollment status to a pharmacy enrolled in the drug distribution management database to authorize the pharmacy to provide to the healthcare setting or the patient a plurality of doses of the drug at a single treatment dose level according to an oral immunotherapy dosing schedule. In some embodiments, the method further comprises repeating steps (a)-(d) at a plurality of different treatment dose levels, wherein the pharmacy is not authorized to simultaneously provide the drug at more than one treatment dose level for the patient.

In some embodiments, a method for authorizing the provision of a drug comprising a food allergen to a pharmacy comprises: (a) verifying enrollment of the pharmacy in a drug distribution management database; (b) authorizing, after enrollment of the pharmacy in the drug distribution management database has been verified, a distributor to provide the drug to the pharmacy.

In some embodiments, a method for authorizing the provision of a drug comprising a food allergen to a pharmacy comprises: (a) receiving a pharmacy enrollment verification request; (b) generating a pharmacy enrollment status indicating whether the pharmacy associated with the pharmacy enrollment verification request is enrolled in a drug distribution management database; (c) sending the pharmacy enrollment status to a distributor enrolled in the drug distribution management database to authorize the distributor to provide the drug to the pharmacy.

In some embodiments, a method for authorizing the distribution of an oral immunotherapy treatment kit comprising a plurality of doses of a drug comprising a food allergen at different dosing levels to a healthcare setting comprises: (a) verifying enrollment of the healthcare setting in a drug distribution management database; (b) authorizing, after enrollment of the healthcare setting in the drug distribution management database has been verified, a distributor to provide the oral immunotherapy treatment kit to the healthcare setting.

In some embodiments, a method for authorizing the distribution of an oral immunotherapy treatment kit comprising a plurality of doses of a drug comprising a food allergen at different dosing levels to a healthcare setting comprises: (a) receiving a healthcare setting enrollment verification request; (b) generating a healthcare setting enrollment status indicating whether the healthcare setting associated with the pharmacy enrollment verification request is enrolled in a drug distribution management database; (c) sending the healthcare setting enrollment status to a distributor enrolled in the drug distribution management database to authorize the distributor to provide the oral immunotherapy treatment kit to the healthcare setting.

In some embodiments, a method for managing the distribution of a drug comprising a food allergen comprises: (a) authorizing the provision of an initial dose escalation kit comprising plurality of different doses of the drug according to an initial dose escalation schedule, comprising: (i) verifying enrollment of a healthcare setting in a drug distribution management database; (ii) verifying enrollment of a patient in a drug distribution management database; (iii) authorizing, after enrollment of the healthcare setting and the patient in the drug distribution management database has been verified, a pharmacy to provide to the healthcare setting the initial dose escalation kit associated with the patient; (b) authorizing the provision of a plurality of doses of the drug at a single treatment dose level according to an oral immunotherapy dosing schedule, comprising: (i) verifying enrollment of a healthcare provider in the drug distribution management database; (ii) verifying enrollment of a patient in the drug distribution management database; (iii) authorizing, after enrollment of the healthcare provider and the patient in the drug distribution management database has been verified, the pharmacy to provide to the healthcare setting or the patient the plurality of doses of the drug at the single treatment dose level; (c) repeating step (b) at a different treatment dose level, wherein the pharmacy is not authorized to simultaneously provide the drug at more than one treatment dose level for the patient; and (d) authorizing the provision of an oral immunotherapy treatment kit comprising a plurality of doses of a drug for treating a food allergy at different up-dosing levels to the healthcare setting, comprising: (i) verifying enrollment of the healthcare setting in the drug distribution management database; (ii) authorizing, after enrollment of the healthcare setting in the drug distribution management database has been verified, a distributor to provide the oral immunotherapy treatment kit to the healthcare setting. In some embodiments, step (a) further comprises verifying enrollment of a healthcare provider in the drug distribution management database, wherein step (a)(iii) occurs after enrollment of the healthcare provider in the drug distribution management database has been verified.

In some embodiments, a method for managing the distribution of a drug comprising a food allergen comprises: A method for managing the distribution of a drug comprising a food allergen, comprising: (a) authorizing the provision of an initial dose escalation kit comprising plurality of different doses of the drug according to an initial dose escalation schedule, comprising: (i) receiving a healthcare setting enrollment verification request and a patient enrollment verification request; (ii) generating a healthcare setting enrollment status indicating whether a healthcare setting associated with the healthcare setting enrollment verification request is enrolled in the drug distribution management database; (iii) generating a patient enrollment status indicating whether a patient associated with the patient enrollment verification request is enrolled in the drug distribution management database; and (iv) sending the healthcare setting enrollment status and the patient enrollment status to a pharmacy enrolled in the drug distribution management database to authorize the pharmacy to provide to the healthcare setting the initial dose escalation kit associated with the patient; (b) authorizing the provision of a plurality of doses of the drug at a single treatment dose level according to an oral immunotherapy dosing schedule, comprising: (i) verifying enrollment of the healthcare provider in the drug distribution management database; (ii) verifying enrollment of the patient in the drug distribution management database; (iii) authorizing, after enrollment of the healthcare provider and the patient in the drug distribution management database has been verified, the pharmacy to provide to the healthcare setting or the patient a plurality of doses of the drug at a single treatment dose level; (c) repeating step (b) at a different treatment dose level, wherein the pharmacy is not authorized to simultaneously provide the drug at more than one treatment dose level for the patient; and (d) authorizing the provision of an oral immunotherapy treatment kit comprising a plurality of doses of a drug for treating a food allergy at different up-dosing levels to the healthcare setting, comprising: (i) receiving a healthcare setting enrollment verification request; (ii) generating a healthcare setting enrollment status indicating whether a healthcare setting associated with the pharmacy enrollment verification request is enrolled in a drug distribution management database; (iii) sending the healthcare setting enrollment status to a distributor enrolled in the drug distribution management database to authorize the distributor to provide the oral immunotherapy treatment kit to the healthcare setting. In some embodiments, step (a) further comprises: receiving a healthcare provider enrollment verification request; generating a healthcare provider enrollment status indicating whether a healthcare provider associated with the healthcare provider enrollment verification request is enrolled in a drug distribution management database; and sending the healthcare provider enrollment status to the pharmacy enrolled in the drug distribution management database to authorize the pharmacy to provide to the healthcare setting an initial dose escalation kit associated with the patient.

In some embodiments of the methods described above, the method further comprises: receiving a pharmacy enrollment request comprising patient information for the patient; and enrolling the pharmacy in the drug distribution management database.

In some embodiments of the methods described above, the method further comprises: receiving a distributor enrollment request comprising patient information for the patient; and enrolling the distributor in the drug distribution management database.

In some embodiments of the methods described above, the method further comprises: receiving a patient enrollment request comprising patient information for the patient; and enrolling the patient in the drug distribution management database.

In some embodiments of the methods described above, the method further comprises: receiving a healthcare setting enrollment request comprising patient information for the patient; and enrolling the healthcare setting in the drug distribution management database.

In some embodiments of the methods described above, the method further comprises: receiving a healthcare provider enrollment request comprising patient information for the patient; and enrolling the healthcare provider in the drug distribution management database.

### BRIEF DESCRIPTION OF THE DRAWINGS

FIG. 1A shows an overview of an exemplary drug distribution system for distribution of the drug containing formulated allergenic material used to treat a food allergy.

FIG. 1B shows an overview of another exemplary drug distribution system for distribution of the drug containing formulated allergenic material used to treat a food allergy.

FIG. 2 illustrates an exemplary oral immunotherapy treatment regimen for treatment of a peanut allergy using a drug containing formulated peanut flour that has been designed and successfully tested in clinical trials.

FIG. 3A illustrates an exemplary initial dose escalation kit that may include doses used in the initial dose escalation phase. FIG. 3B shows the inside portion of the initial dose escalation kit.

FIG. 4 shows an exemplary oral immunotherapy treatment kit that may be in the possession of a healthcare setting enrolled in the drug distribution management database.

FIG. 5 provides an overview of exemplary distribution controls for the drug distribution methods described herein.

FIG. 6 shows an exemplary process for providing the initial dose escalation kit containing the drug.

FIG. 7 illustrates an exemplary method for providing the oral immunotherapy treatment kit to a healthcare setting.

FIG. 8 shows an exemplary process for distributing the plurality of doses of the drug at the selected dose level is by a pharmacy.

FIG. 9 illustrates an example of a computing device that may be used for the methods or systems described herein.

FIG. 10 provides another overview of exemplary distribution controls for the drug distribution methods described herein.

### DETAILED DESCRIPTION OF THE INVENTION

Successful treatment of a food allergy in a patient by oral immunotherapy (OIT) desensitizes the patient to allergens in the food, which can decrease the risk of a systemic or severe allergic reaction, such as anaphylaxis, upon dietary consumption of the food. Completion of therapy may not result in an absolute ablation of allergic response, but generally increases the threshold amount of allergenic material needed to induce a life-threatening reaction. During the administration of a drug containing allergenic proteins for treating a food allergy in a patient by oral immunotherapy, mild or moderate allergic reactions can be expected. Occasionally, treatment can results in severe or systemic allergic reaction, such as anaphylaxis, a potentially life-threatening response to the ingestion of the allergenic composition. The oral immunotherapy treatment regimen is further complicated by numerous dosing phases and dosing levels, as further described herein, and it is important that a patient receive the correct dose at each administration to limit the allergic response risk. Unregulated distribution or administration of drugs for oral immunotherapy can have potentially fatal consequences. The processes and systems described herein are implemented for carefully regulated administration and distribution of the drug by requiring certain checkpoints in the administration or distribution process. As further described herein, systems and methods have been developed that carefully control the distribution and administration of the allergenic drug for oral immunotherapy to mitigate risk associated with allergic responses to the drug.

Distribution and administration of the drug can include multiple touchpoints between manufacturers, distributors, pharmacies, healthcare settings, prescribers (such as healthcare providers), and patients. Additionally, the drug may be distributed or administered in different formats during oral immunotherapy, such as an initial dose escalation kit, an oral immunotherapy treatment kit used in a healthcare setting, up-dosing dosage forms that are distributed or administered at different dosing levels, and maintenance dosage forms. Any one or more of these dosage forms may be used to treat the patient during the oral immunotherapy treatment regimen, and the distribution and administration of the drug must be carefully controlled to ensure the patient receives the correct amount of drug at the correct time and in the correct setting. For example, initial dose escalation, administration of the first dose of each up-dosing level, and the first maintenance dose are performed in a supervised healthcare setting. Only after a dose level is successfully administered to the patient can distribution of the drug at that level to the patient be authorized.

The methods and systems described herein allow for the enrollment of distributors, pharmacies, healthcare settings, prescribers (such as healthcare providers), and/or patients to be enrolled in a drug distribution management database for the drug. Enrollment of these entities in the drug distribution management database ensures that only authorized entities receive or distribute the drug, which limits the risk that the incorrect dose of the drug is administered to the patient. Distribution of the drug is contingent on ensuring enrollment of the distributing entity and enrollment of the receiving entity on being enrolled in the drug distribution management database.

### Definitions

As used herein, the singular forms “a,” “an,” and “the” include the plural references unless the context clearly dictates otherwise.

Reference to “about” a value or parameter herein includes (and describes) variations that are directed to that value or parameter per se. For example, description referring to “about X” includes description of “X”.

The term “desensitized” is used herein to refer to an increased reaction threshold to a food allergen by a subject as a result of an oral immunotherapy for the food allergen. Desensitization to a food allergen can be tested using methods known in the art, including an oral food challenge. Desensitization may be partial, wherein the subject tolerates an increased amount of the food allergen compared to prior to treatment, but still reacts to higher doses of the food allergen; or the desensitization may be complete, wherein the patient tolerates all tested doses of the food allergen.

The terms “effective,” “efficacy,” or “effectiveness” are used herein to refer to the ability of a therapy to induce immune modulation, such as desensitization, or sustain a desired immune state, such as a desensitized state, unless otherwise indicated.

As described herein, an action performed or carried out by a “healthcare setting” may be performed or carried out by the healthcare setting itself or a representative of the healthcare setting, such as a healthcare provider (e.g., doctor, nurse, nurse practitioner, etc.) associated with or acting on behalf of the healthcare setting. By way of example, “a prescription issued by a healthcare setting” encompasses a prescription issued by a healthcare provider associated with or acting on behalf of the healthcare setting.

The term “maintenance phase” refers to a phase of oral immunotherapy that includes administration of a food allergen (i.e., a maintenance dose) to the patient, and occurs after completion of the up-dosing phase.

The term “mild allergic adverse event” or “mild allergic reaction” refers to an observed or experienced OIT-treatment-related allergic adverse event associated with transient discomfort, but does not require immediate medical intervention such as hospitalization or epinephrine, and does not substantially interfere with daily activities.

The term “moderate allergic adverse event” or “moderate allergic reaction” refers to an observed or experienced OIT-treatment-related allergic adverse event that is associated with discomfort of a sufficient degree to interfere with daily activities and that may prompt medical intervention and/or additional observation.

The term “serious allergic adverse event” or “serious allergic reaction” refers to an observed or experienced OIT-treatment-related allergic adverse event leading to anaphylaxis that requires hospitalization and/or administration of epinephrine or other life-saving medical intervention.

The term “systemic allergic adverse event” or “systemic allergic reaction” refers to an observed or experienced OIT-treatment-related allergic adverse event that affect two or more body systems.

The term “subject” or “patient” is used synonymously herein to describe a human of any age.

A subject “tolerates” a dose when the dose is administered to the subject without any moderate or severe (i.e., serious) allergic adverse event. A subject is considered to tolerate the dose even if a mild allergic adverse event is observed or experienced.

The terms “treat,” “treating,” and “treatment” are used synonymously herein to refer to any action providing a benefit to a subject afflicted with a disease state or condition, including improvement in the condition through lessening, inhibition, suppression, or elimination of at least one symptom; delay in progression of the disease; delay in recurrence of the disease; inhibition of the disease; or partially or fully reducing a response or reaction to an allergen.

An “up-dosing phase” refers to a phase of an oral immunotherapy characterized by a series of increasing food allergen doses, beginning with administration of a dose of food allergen lower than the highest dose administered to the patient during the oral immunotherapy, and ending when the highest dose administered to the patient during the oral immunotherapy is achieved.

It is understood that aspects and variations of the invention described herein include “consisting” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The disclosures of all publications, patents, and patent applications referred to herein are each hereby incorporated by reference in their entireties. To the extent that any reference incorporated by reference conflicts with the instant disclosure, the instant disclosure shall control.

### Drug Distribution Management System Overview

Patients, healthcare settings, prescribers (such as healthcare providers), pharmacies, and distributors are enrolled in a drug distribution management database, and entities that provide the drug to another entity must ensure that the entity receiving the drug is enrolled in the drug distribution management database. The providing entity can submit an enrollment verification request, which is received by an electronic device. The electronic device then accesses the drug distribution management database and compares information provided in the enrollment verification request to information in the drug distribution management database to determine whether the queried entity is enrolled in the drug distribution management database. If the queried entity is enrolled, a positive enrollment status is transmitted to the requesting entity, which authorizes the requesting entity to provide the drug to the queried entity. Additional details of these drug distribution management methods are provided herein.

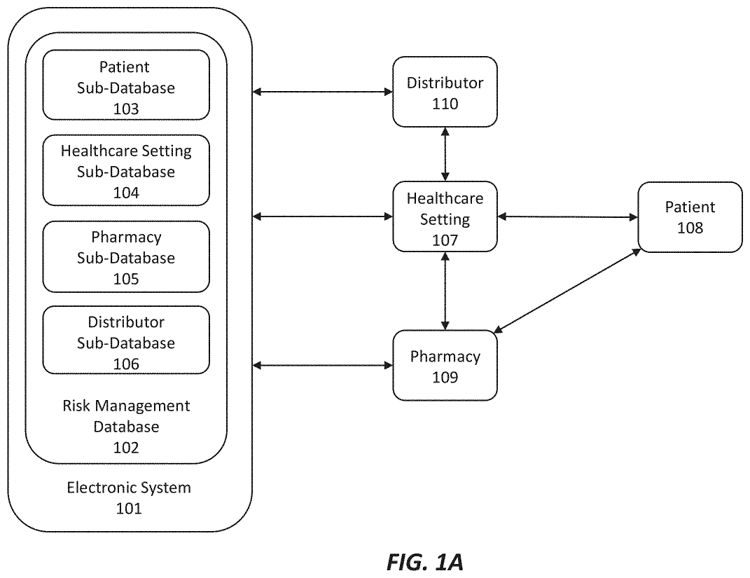

FIG. 1A shows an overview of an exemplary drug distribution management system of the drug containing formulated allergenic material (such as allergenic peanut flour) used to treat a food allergy (e.g., a peanut allergy). An electronic system 101 includes a memory that stores a drug distribution management database 102, which includes sub-databases, including an enrolled patient sub-database 103, an enrolled healthcare setting sub-database 104, an enrolled pharmacy sub-database 105, and an enrolled distributor database 106. The enrolled patient sub-database 103 includes a plurality of entries (which may be organized as a list, nodes, or other suitable format) for patients enrolled in the sub-database, with each entry containing patient information (such as one or more of a patient name, an enrollment number, an address, birthday, sex, telephone number, parent or guardian name, healthcare setting information identifying the healthcare setting treating the patient for the food allergy, and/or a confirmation that the patient and/or guardian has read and/or understood patient attestations about the use of the drug). The enrolled healthcare setting sub-database 104 includes a plurality of entries (which may be organized as a list, nodes, or other suitable format) for healthcare settings enrolled in the sub-database, with each entry containing healthcare setting information (such as one or more of a healthcare setting name, an enrollment number, the name of one or more authorized representatives of the healthcare setting, the name and/or medical license number of one or more healthcare providers associated with the healthcare setting, an address, a telephone number, a list of patients being treated with the drug by the healthcare setting, and/or a confirmation that an authorized representative has read and/or understood the healthcare setting attestations about the use of the drug). The enrolled pharmacy sub-database 105 includes a plurality of entries (which may be organized as a list, nodes, or other suitable format) for pharmacies enrolled in the sub-database, with each entry containing pharmacy information (such as one or more of the name of the pharmacy, an enrollment number, an address, a telephone number, the name of one or more authorized representatives of the pharmacy, a pharmacy license number, and/or a confirmation that an authorize representative has read and/or understood pharmacy attestations about providing the drug to another entity). The enrolled distributor sub-database 106 includes a plurality of entries (which may be organized as a list, nodes, or other suitable format) for distributors enrolled in the sub-database, with each entry containing distributor information (such as one or more of a distributor name, an enrollment number, a distributor license number, an address, a telephone number, the name of one or more authorized representatives of the distributor, and/or a confirmation that an authorized representative has read and/or understood distributor attestations about providing the drug to another entity).

A healthcare setting 107 can be enrolled in the healthcare setting sub-database 104 by submitting a healthcare setting enrollment request to a drug distribution management database manager. The healthcare setting enrollment request includes healthcare information, and the drug distribution management database manager can approve enrollment and/or generate an entry for the healthcare setting 107 in the healthcare setting sub-database 104. Once the healthcare setting 107 is enrolled in the healthcare setting sub-database may submit, on behalf of a patient 108, a patient enrollment request containing patient information to the drug distribution management database manager, which can approve enrollment and/or generate an entry for the patient 108 in the patient sub-database 103. Alternatively, the healthcare setting 107 may be authorized to generate an entry for the patient 108 in the patient sub-database 103.

A pharmacy 109 can be enrolled in the pharmacy sub-database 105 by submitting a pharmacy enrollment request to a drug distribution management database manager. The pharmacy enrollment request includes pharmacy information, and the drug distribution management database manager can approved enrollment and/or generate an entry for the pharmacy 108 in the pharmacy sub-database 105. Once a pharmacy 109 is enrolled in the pharmacy sub-database 105, the pharmacy can submit a patient enrollment verification request or a healthcare setting enrollment verification request to the electronic system 101 or the drug distribution management database manager to verify enrollment of the patient and/or healthcare setting. The pharmacy 109 enrolled in the pharmacy sub-database 105 is authorized to distribute the drug to the healthcare setting 107 (for the initial dose escalation kit and/or up-dosing doses at a selected level) or the patient 108 (for up-dosing doses at the selected level) only after enrollment of the patient 108 and the healthcare setting 107 has been verified.

A distributor 110 can be enrolled in the distributor sub-database 106 by submitting a distributor enrollment request to a drug distribution management database manager. The distributor enrollment request includes distributor information, and the drug distribution management database manager can approved enrollment and/or generate an entry for the distributor 110 in the distributor sub-database 106. Once the distributor 110 is enrolled in the distributor sub-database 106, the distributor can submit a healthcare setting enrollment verification request to the electronic system 101 or the drug distribution management database manager to verify enrollment of the healthcare setting. The distributor 110 enrolled in the distributor sub-database 110 is authorized to distribute the drug, packaged in an oral immunotherapy treatment kit, to the healthcare setting 107 only after enrollment of the healthcare setting 107 has been verified.

FIG. 1B shows an overview of another exemplary drug distribution management system of the drug containing formulated allergenic material (such as allergenic peanut flour) used to treat a food allergy (e.g., a peanut allergy). An electronic system 151 includes a memory that stores a drug distribution management database 152, which includes sub-databases, including an enrolled patient sub-database 153, an enrolled prescribed sub-database 154 (e.g., an enrolled healthcare provider sub-database), an enrolled healthcare setting sub-database 155, an enrolled pharmacy sub-database 156, and an enrolled distributor database 157. The enrolled patient sub-database 153 includes a plurality of entries (which may be organized as a list, nodes, or other suitable format) for patients enrolled in the sub-database, with each entry containing patient information (such as one or more of a patient name, an enrollment number, an address, birthday, sex, telephone number, parent or guardian name, healthcare setting information identifying the healthcare setting treating the patient for the food allergy, and/or a confirmation that the patient and/or guardian has read and/or understood patient attestations about the use of the drug). The enrolled prescriber sub-database 154 includes a plurality of entries (which may be organized as a list, nodes, or other suitable format) for prescribers (e.g., healthcare providers) enrolled in the sub-database, with each entry containing prescriber information (such as one or more of a prescriber name, an enrollment number, an identification number (e.g., a National Provider Identifier, a medical license number, etc.), an address, a telephone number, a list of patients being treated with the drug by the prescriber, the name of an associated healthcare setting, and/or confirmation that a prescriber has read and/or understood the prescriber attestations about the use of the drug). The enrolled healthcare setting sub-database 155 includes a plurality of entries (which may be organized as a list, nodes, or other suitable format) for healthcare settings enrolled in the sub-database, with each entry containing healthcare setting information (such as one or more of a healthcare setting name, an enrollment number, the name of one or more authorized representatives of the healthcare setting, the name and/or medical license number of one or more healthcare providers associated with the healthcare setting, an address, a telephone number, a list of patients being treated with the drug by the healthcare setting, and/or a confirmation that an authorized representative has read and/or understood the healthcare setting attestations about the use of the drug). The enrolled pharmacy sub-database 156 includes a plurality of entries (which may be organized as a list, nodes, or other suitable format) for pharmacies enrolled in the sub-database, with each entry containing pharmacy information (such as one or more of the name of the pharmacy, an enrollment number, an address, a telephone number, the name of one or more authorized representatives of the pharmacy, a pharmacy license number, and/or a confirmation that an authorize representative has read and/or understood pharmacy attestations about providing the drug to another entity). The enrolled distributor sub-database 157 includes a plurality of entries (which may be organized as a list, nodes, or other suitable format) for distributors enrolled in the sub-database, with each entry containing distributor information (such as one or more of a distributor name, an enrollment number, a distributor license number, an address, a telephone number, the name of one or more authorized representatives of the distributor, and/or a confirmation that an authorized representative has read and/or understood distributor attestations about providing the drug to another entity).

A prescriber 159 can be enrolled in the prescriber sub-database 154 by submitting a prescriber enrollment request to a drug distribution management database manager. The prescriber enrollment request includes prescriber information, and the drug distribution management database manager can approve enrollment and/or generate an entry for the prescriber 159 in the prescriber sub-database 154.

A healthcare setting 158 can be enrolled in the healthcare setting sub-database 155 by submitting a healthcare setting enrollment request to a drug distribution management database manager. The healthcare setting enrollment request includes healthcare setting information, and the drug distribution management database manager can approve enrollment and/or generate an entry for the healthcare setting 158 in the healthcare setting sub-database 155. Once the healthcare setting 158 is enrolled in the healthcare setting sub-database may submit, on behalf of a patient 160, a patient enrollment request containing patient information to the drug distribution management database manager, which can approve enrollment and/or generate an entry for the patient 160 in the patient sub-database 153. Alternatively, the healthcare setting 158 may be authorized to generate an entry for the patient 160 in the patient sub-database 153.

A pharmacy 161 can be enrolled in the pharmacy sub-database 156 by submitting a pharmacy enrollment request to a drug distribution management database manager. The pharmacy enrollment request includes pharmacy information, and the drug distribution management database manager can approved enrollment and/or generate an entry for the pharmacy 161 in the pharmacy sub-database 156. Once a pharmacy 161 is enrolled in the pharmacy sub-database 156, the pharmacy can submit a patient enrollment verification request, a prescriber enrollment verification request, and/or a healthcare setting enrollment verification request to the electronic system 151 for the drug distribution management database manager to verify enrollment of the patient, prescriber, and/or healthcare setting. In some embodiments, the pharmacy 161 enrolled in the pharmacy sub-database 156 is authorized to distribute the drug to the healthcare setting 158 or the prescriber 159 (for the initial dose escalation kit and/or up-dosing doses at a selected level) or the patient 160 (for up-dosing doses at the selected level) only after enrollment of the patient 160, the prescriber 159, and the healthcare setting 158 has been verified. In some embodiments, the pharmacy 161 enrolled in the pharmacy sub-database 156 is authorized to distribute the drug to the healthcare setting 158 or the prescriber 159 (for the initial dose escalation kit and/or up-dosing doses at a selected level) or the patient 160 (for up-dosing doses at the selected level) only after enrollment of the patient 160 and the prescriber 159 has been verified.

A distributor 162 can be enrolled in the distributor sub-database 157 by submitting a distributor enrollment request to a drug distribution management database manager. The distributor enrollment request includes distributor information, and the drug distribution management database manager can approved enrollment and/or generate an entry for the distributor 162 in the distributor sub-database 157. Once the distributor 162 is enrolled in the distributor sub-database 157, the distributor can submit a healthcare setting enrollment verification request to the electronic system 151 for the drug distribution management database manager to verify enrollment of the healthcare setting. The distributor 162 enrolled in the distributor sub-database 157 is authorized to distribute the drug, packaged in an oral immunotherapy treatment kit, to the healthcare setting 158 only after enrollment of the healthcare setting 158 has been verified.

### Treated Patients

The patients can be treated for a food allergy, such as a peanut allergy, using the oral immunotherapy methods described in further detail herein. Briefly, treatment generally includes an initial dose escalation phase (which, in some embodiments, is omitted), an up-dosing phase that includes periodic escalation of the drug dose as a plurality of different dose levels, and a maintenance phase. The patient is treated under the supervision of a healthcare provider associated with a healthcare setting. The drug distribution management systems and methods described herein are designed to mitigate risk to the patient by ensuring the patient receives the correct amount of the drug at the correct time during the course of oral immunotherapy treatment.

Prior to treatment, the patient is enrolled in the patient sub-database of the drug distribution management database. A patient enrollment request is submitted to the drug distribution system manager, for example by a healthcare setting or authorized representative of the healthcare setting, such as a healthcare provider (e.g., prescriber). The patient enrollment request may be a form that is electronically transmitted (for example, through a secure network, internet, IP protocol, a web interface, mobile application (e.g., a mobile phone or tablet application), electronic mail, electronic records transfer, faxed, or any other suitable electronic transmission). The patient enrollment request includes patient information, such as the name of the patient, the birthdate of the patient, the sex of the patient, contact information for the patient (such as address, telephone number, email address, etc.), a parent or guardian name, and/or parent or guardian contact information. The patient enrollment request may also include a confirmation (which may be indicated, for example, by a signature or initial) that the patient and/or the guardian has read and/or understood patient attestations. Such patient attestations may include, but are not limited to, any one or more of the following: (1) that the patient has been instructed to have immediate access to and/or carry an anaphylaxis-response treatment (such as epinephrine, albuterol, diphenhydramine, etc.) during the course of treatment for the food allergy, (2) the patient has been instructed to avoid dietary consumption of the food associated with the food allergy for which the patient is receiving treatment, (3) the patient has been instructed about the signs and/or symptoms of an allergic reaction, (4) the patient agrees to remain at the healthcare setting for a monitoring period (e.g., an hour) after receiving a dose of the drug at the healthcare setting to be monitored for an allergic response, (5) the patient agrees to report an allergic reaction to the healthcare setting or authorized representative of the healthcare setting, (6) the patient agrees to inform the healthcare setting or authorize representative of the healthcare setting if additional anaphylaxis-response treatment medication is needed, and/or (7) the patient agrees to have immediate access to and/or carry an anaphylaxis-response treatment during the course of treatment for the food allergy. The patient information may also include information about the healthcare setting that is treating the patient for the food allergy, such as the name of the healthcare setting, a registration number (such as a national provider identifier, an enrollment number, or other alphanumeric identifier), and/or contact information for the healthcare setting (e.g., an address, a telephone number, a fax number, and/or an email address).

The healthcare setting treating the patient can transmit the patient enrollment request to the drug distribution management system manager. Once received by the drug distribution management system manager, an entry for the patient can be generated in the enrolled patient sub-database of the drug distribution management database, which includes the patient information. Once enrolled in the patient sub-database, the drug distribution management system manager can send a confirmation of enrollment to the healthcare setting and/or patient.

In some embodiments, the prescriber (such as the healthcare provider), which may be associated with the healthcare setting, treating the patient can transmit the patient enrollment request to the drug distribution management system manager. Once received by the drug distribution management system manager, an entry for the patient can be generated in the enrolled patient sub-database of the drug distribution management database, which includes the patient information. Once enrolled in the patient sub-database, the drug distribution management system manager can send a confirmation of enrollment to the prescriber (such as the healthcare provider) and/or patient.

In some embodiments, the patient information in the patient sub-database of the drug distribution management database is updated to indicate a dose level for the drug being used to treat the patient. The dose level may be updated by a pharmacy enrolled in the drug distribution management database or by a healthcare setting enrolled in the drug distribution management database. For example, if the pharmacy provides an initial dose escalation kit to a healthcare setting (or a prescriber, such as a healthcare provider) for a particular patient, the patient information for that patient may be updated to indicate that the patient is being treated with the initial dose escalation kit. If the pharmacy dispenses a plurality of doses of a drug at a selected dose level, for example as prescribed by a healthcare setting or healthcare provider, the patient information can be updated by the pharmacy to indicate the dose level being used to treat the patient.

### Healthcare Providers

Prescribers (also referred to as healthcare providers) treat and/or supervise the treatment of patients for the food allergy, and must be enrolled in the prescriber sub-database of the drug distribution management database before initiating treatment or receiving an oral immunotherapy treatment kit or an initial dose escalation kit. Responsibilities of the healthcare provider are further described herein. In brief, the healthcare provider administers the doses (including on behalf of the healthcare setting) contained with the initial dose escalation kit (which may be received from a pharmacy and is specifically associated with the patient once the patient is enrolled in the drug distribution management database, as further described herein) to the patient (assuming the initial dose escalation kit is used in the oral immunotherapy regimen, which is optional in some embodiments). Between initial escalation doses, the healthcare provider monitors the patient (including on behalf of the healthcare setting) for an allergic reaction during a monitoring period. The healthcare provider is further responsible for administering the first dose (including on behalf of the healthcare setting) at each dose level to the patient. The healthcare provider administers (or supervises the administration of) the first dose at a given dose level and monitors (or supervises the monitoring of) the patient for an allergic response on behalf of the healthcare setting. The healthcare provider may then prescribe that dose level to the patient. To change the dose level, the patient must return to the healthcare provider at the associated healthcare setting and the healthcare provider administers a dose of the subsequent dose level and monitors the patient for an allergic response during the monitoring period. This process is repeated for each dose level during the up-dosing and maintenance phases of the oral immunotherapy.

The healthcare provider (or the healthcare setting) can transmit a healthcare provider enrollment request to the drug distribution management database manager, and, if approved, the drug distribution management database manager can generate an entry in the prescriber sub-database of the drug distribution management database that includes prescriber (i.e., healthcare provider) information to enroll the healthcare provider in the drug distribution management database. The healthcare provider enrollment request may be a form that is electronically transmitted (for example, through a secure network, a web interface, electronic mail, electronic records transfer, faxed, or any other suitable electronics transmission). Healthcare provider information can include one or more of a healthcare provider name, an identification number (e.g., a National Provider Identifier, a license number, etc.), contact information for the healthcare provider (e.g., an address, a telephone number, a fax number, and/or an email address), information for one or more healthcare settings that the healthcare provider is associated with (such as healthcare setting address, telephone number, fax number, name, etc.), and/or confirmation (such as a signature) of the healthcare provider attestations. The healthcare provider attestations may include, but are not limited to, one or more of (1) a confirmation that the healthcare provider will, before treatment initiation and/or before writing a prescription to a patient, will enroll each patient (or confirm the enrollment) of a patient in the drug distribution management database, (2) a confirmation that the healthcare provider will, before treatment initiation and/or before writing a prescription to a patient, will provide a copy of a completed patient enrollment form to the patient, (3) a confirmation that the healthcare provider will, before treatment initiation and/or before writing a prescription to a patient, will counsel the patient on the need to have injectable epinephrine available for immediate uses, the need for monitoring during the initial dose escalation (if part of the oral immunotherapy), the need for monitoring the first dose of each up-dosing level, the need for continued peanut avoidance in the diet, and how to recognize the signs and symptoms of anaphylaxis, (4) a confirmation that the healthcare provider will, before treatment initiation and/or before writing a prescription to a patient, will assess the patient's supply of injectable epinephrine and provide a prescription for epinephrine if necessary, (5) a confirmation that the healthcare provider will, during treatment but before dispensing the first dose of each up-dosing level, will assess the patient's tolerability of the previous dosing level and appropriateness of continuing the up-dosing according to the oral immunotherapy schedule, and (6) a confirmation that, at all times, the healthcare provider will report treatment discontinuation or transfer of care of the patient to the healthcare management database manager.

### Healthcare Settings

Healthcare settings oversee patient treatment of the food allergy, and must be enrolled in the healthcare setting sub-database of the drug distribution management database before initiating treatment or receiving an oral immunotherapy treatment kit or an initial dose escalation kit. Responsibilities of the healthcare setting for treating the patient are further described herein. In brief, the healthcare setting administers the doses contained with the initial dose escalation kit (which may be received from a pharmacy and is specifically associated with the patient once the patient is enrolled in the drug distribution management database, as further described herein) to the patient (assuming the initial dose escalation kit is used in the oral immunotherapy regimen, which is optional in some embodiments). Between initial escalation doses, the healthcare setting monitors the patient for an allergic reaction during a monitoring period. The healthcare setting is further responsible for administering the first dose at each dose level to the patient. As further described herein, the oral immunotherapy treatment kit at the healthcare setting includes a plurality of doses at each dose level used during the oral immunotherapy. The healthcare setting (or authorized representative) administers the first dose at a given dose level and monitors the patient for an allergic response. The healthcare setting may then prescribe that dose level to the patient. To change the dose level, the patient must return to the healthcare setting and the healthcare setting administers a dose of the subsequent dose level and monitors the patient for an allergic response during the monitoring period. This process is repeated for each dose level during the up-dosing and maintenance phases of the oral immunotherapy.

The healthcare setting can transmit a healthcare setting enrollment request to the drug distribution management database manager, and, if approved, the drug distribution management database manager can generate an entry in the healthcare setting sub-database of the drug distribution management database that includes healthcare setting information to enroll the healthcare setting in the drug distribution management database. The healthcare setting enrollment request may be a form that is electronically transmitted (for example, through a secure network, a web interface, electronic mail, electronic records transfer, faxed, or any other suitable electronic transmission). Healthcare setting information can include one or more of a healthcare setting name, an identification number (e.g., a national provider identifier, a Drug Enforcement Agency (DEA) number, etc.), contact information for the healthcare setting (e.g., an address, a telephone number, a fax number, and/or an email address, etc.), a name of an authorized representative, and/or confirmation (such as a signature) of the healthcare setting attestations. The healthcare setting attestations may include, but are not limited to, one or more of (1) a confirmation that an authorized representative and/or treating healthcare providers have reviewed educational information about using the drug to treat food allergy, (2) a confirmation that a patient administered a dose of the drug at the healthcare setting will be monitored for an allergic response during a monitoring period, (3) a confirmation that the healthcare setting is equipped to treat a patient for an allergic response (such as a systemic allergic response and/or anaphylaxis), (4) a confirmation that the healthcare setting will establish a practice to have patients enrolled in the drug distribution management database and/or verify enrollment of patients in the drug distribution management database, (5) a confirmation that an initial dose escalation kit received by the healthcare setting will only be used for the patient specifically associated with the initial dose escalation kit, (6) a confirmation that the healthcare setting will have a process to ensure a patient being treated for the food allergy by the healthcare setting has access to an anaphylaxis-response treatment (such as epinephrine, albuterol, diphenhydramine, etc.) during the course of treatment for the food allergy, (7) a confirmation that the healthcare setting will instruct the patient being treated for the food allergy to avoid dietary consumption of the food associated with the food allergy being treated, (8) a confirmation that the healthcare setting will instruct the patient how to recognize the signs and/or symptoms of an allergic response, (9) a confirmation that the healthcare setting will inform the patient and/or parent or guardian of the patient being treated that the patient will need to be monitored for an allergic response during a monitoring period after administration of the drug in the healthcare setting, (10) a confirmation that the healthcare setting will not distribute, transfer, loan, or sell the drug, (11) a confirmation that the healthcare setting will not distribute the initial dose escalation kit for use outside of the healthcare setting, (12) a confirmation that that doses from the oral immunotherapy kit will not be administered a patient not enrolled in drug distribution management system, and/or (13) a confirmation that the healthcare setting will only initiate treatment using an initial dose escalation kit.

### Pharmacies

Pharmacies enrolled in the pharmacy sub-database of the drug distribution management database distribute (1) the initial dose escalation kit associated with a specific patient enrolled in the patient sub-database of the drug distribution management database to a healthcare setting enrolled in the healthcare setting sub-databases of the drug distribution management database and/or (2) doses of the drug at a prescribed dose level to a patient enrolled in the patient sub-database of the drug distribution management database or a healthcare setting enrolled in the healthcare setting sub-database. In some embodiments, prior to distributing the drug, the pharmacy is required to verify enrollment of the patient and the healthcare setting in the drug distribution management database. In some embodiments, prior to distributing the drug, the pharmacy is required to verify enrollment of the patient and the healthcare provider (and/or the healthcare setting that the healthcare provider is associated with) in the drug distribution management database. In some embodiments, prior to distributing the drug, the pharmacy is required to verify enrollment of the patient, the healthcare setting, and the healthcare provider in the drug distribution management database.

A pharmacy can be enrolled in the pharmacy sub-database by transmitting a pharmacy enrollment request to the drug distribution management database manager. The pharmacy enrollment request may be a form that is electronically transmitted (for example, through a secure network, a web interface, electronic mail, electronic records transfer, faxed, or any other suitable electronic transmission). Once the drug distribution management database manager receives the enrollment request, the drug distribution management database manager may authorize enrollment of the pharmacy and/or generate an entry for the pharmacy in the pharmacy sub-database that includes information about the pharmacy. The pharmacy enrollment request includes pharmacy information, such as the pharmacy name, an identification number (such as a National Provider Identifier, a National Council for Prescription Drug Program ID, a Drug Enforcement Agency (DEA) number, etc.), contact information for the pharmacy (such as an address, a telephone number, a fax number, and/or an email address), a name or identifying information for an authorized representative of the pharmacy, and/or confirmation (such as a signature) of pharmacy attestations. The pharmacy attestations may include, for example, but not limited to, one or more of (1) a confirmation that the authorized representative has reviewed an overview of the drug distribution management system, (2) a confirmation that an initial dose escalation kit will be associated with a specific patient enrolled in the patient sub-database of the drug distribution management database, (3) a confirmation that the initial dose escalation kit will be provided only to a healthcare setting enrolled in the healthcare setting sub-database, (4) a confirmation that the pharmacy will establish a process to ensure that only a single dose level of the drug will be distributed to the patient at any given time, (5) a confirmation that the pharmacy will verify enrollment of the patient and the healthcare setting in the drug distribution management database before distributing the drug (either as the initial dose escalation kit or the up-dosing or maintenance level) to the healthcare setting or patient, and/or (6) a confirmation that the pharmacy will maintain a record for the patient that indicates the dose level of the drug being administered to the patient.

The pharmacy can transmit a healthcare setting enrollment verification request and a patient verification request to the drug distribution management database manager to ensure the healthcare setting and the patient are enrolled in the drug distribution management database. The pharmacy can also transmit a prescriber (such as a healthcare provider) enrollment verification request to the drug distribution management database manager to ensure the prescriber (such as a healthcare provider) is enrolled in the drug distribution management database. The prescriber enrollment verification request can include prescriber (such as healthcare provider) information, which is used to compare information in entries in the database and generate an enrollment status for the prescriber (such as a healthcare provider). The healthcare setting enrollment verification request can include healthcare setting information, and the patient enrollment verification request can include patient information, which are used to compare to information in entries in the database and generate an enrollment status for the patient and healthcare setting. In some embodiments, a positive enrollment status for the patient and healthcare setting authorizes the pharmacy, which must also be enrolled in the drug distribution management database, to distribute the drug to the patient and/or healthcare setting. In some embodiments, a positive enrollment status for the patient and the prescriber authorizes the pharmacy, which must also be enrolled in the drug distribution management database, to distribute the drug to the patient and/or healthcare setting. In some embodiments, a positive enrollment status for the patient, the prescriber, and the healthcare setting authorizes the pharmacy, which must also be enrolled in the drug distribution management database, to distribute the drug to the patient and/or healthcare setting.

### Distributors

Distributors that are enrolled in the distributor sub-database of the drug distribution management database can provide the oral immunotherapy treatment kit, which is used to provide the first dose at each dose level during the up-dosing phase of the oral immunotherapy and the first maintenance dose during the maintenance phase of the oral immunotherapy, to a healthcare setting enrolled in the healthcare setting enrolled in the healthcare setting sub-database of the drug distribution management database. The distributor is required to verify that the healthcare setting is enrolled in the drug distribution management database prior to providing the oral immunotherapy treatment kit to the healthcare setting.

A distributor can be enrolled in the distributor sub-database of the drug distribution management database by transmitting a distributor enrollment request to the drug distribution management database manager. Upon receipt of the enrollment request, the drug distribution management database manager may authorize enrollment for the distributor and/or generate an energy for the distributor in the distributor sub-database that includes information about the distributor. The distributor enrollment request may be a form that is electronically transmitted (for example, through a secure network, a web interface, electronic mail, electronic records transfer, faxed, or any other suitable electronic transmission). Distributor information that is included in the enrollment request may include, for example, one or more of the distributor name, a distributor identifier (e.g., a National Provider Identifier, a Distributor Drug Enforcement Agency number, or some other alphanumeric identifier), contact information about the distributor (such as an address, a telephone number, a fax number, and/or an email address), and/or a confirmation (such as a signature) of distributor attestations. The distributor attestations can include, for example, one or more of (1) a confirmation that the distributor will verify enrollment of healthcare settings before providing the drug to the healthcare setting, (2) a confirmation that the distributor will verify enrollment of a pharmacy before providing the drug to the pharmacy, and/or (3) a confirmation that the distributor will maintain records that indicate provision of the drug to a healthcare setting and/or pharmacy.

Prior to providing the drug (such as an oral immunotherapy treatment kit) to a healthcare setting, the distributor is required to verify that the healthcare setting is enrolled in the healthcare setting sub-database of the drug distribution management database. The distributor transmits a healthcare setting enrollment verification request containing healthcare setting information to the drug distribution management database manager. The drug distribution management database manager can then compare the healthcare setting information included in the verification request to the healthcare setting information contained within entries in the healthcare setting sub-database, and generate an enrollment status that is transmitted to the distributor. A positive healthcare setting enrollment status received by the distributor authorizes the distributor to provide the drug (e.g., the oral immunotherapy treatment kit) to the healthcare setting.

### Oral Immunotherapy

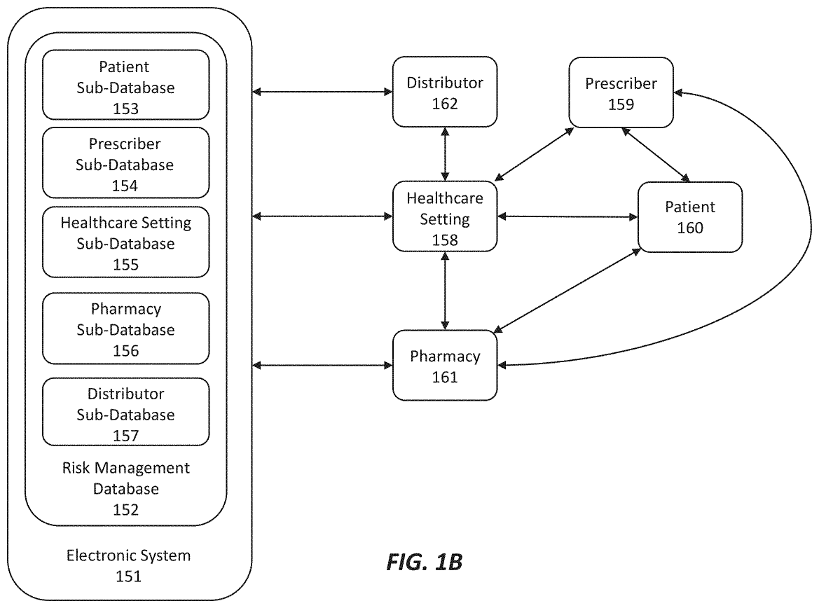

A patient having a food allergy can be treated for the food allergy by administering a series of doses of a drug containing allergenic proteins formulated with one or more excipients, to the patient during the course of an oral immunotherapy treatment regimen in accordance with a dosing schedule, thereby desensitizing the patient to the food allergy. The oral immunotherapy treatment regimen is multi-phasic, and include an initial dose escalation (IDE) phase (which, in some embodiments, may be omitted form the treatment regimen), an up-dosing phase, and a maintenance phase. FIG. 2 illustrates an exemplary oral immunotherapy treatment regimen for treatment of a peanut allergy using a drug containing formulated peanut flour that has been designed and successfully tested in clinical trials.

Oral immunotherapy can be initiated using an initial dose escalation phase, wherein the patient is administered over the course of one or two days a series of escalating doses. This series of initial doses is administered in a healthcare setting so that the patient may be monitored for an allergic reaction after ingesting the doses. A monitoring period follows the administration of each dose, and if the patient does not have a severe adverse reaction, the subsequent dose may be administered and the patient monitored. Because administration of a drug containing food allergens to a patient allergic to that food risks an allergic response, the drug distribution management system described herein is designed to ensure the initial dose escalation is administered only in the healthcare setting where the patient can be monitored. The initial dose escalation phase is discussed in further detail herein.

After the initial dose escalation phase (if included in the treatment regimen), the patient enters the up-dosing phase. During the up-dosing phase of the oral immunotherapy, the dose strength (or “level”) of the drug administered to the patient is periodically increased until a target dose level is reached. For example, during the up-dosing phase, the patient may be administered the same amount of peanut protein composition on a daily basis for a period of time (for example, 1, 2, 3, 4 or more weeks), before the dose level is increased. As increasing the amount of the drug administered to the patient increases the risk of a severe allergic response, the drug distribution management system described herein is designed to ensure the first dose of each increased level is administered in a healthcare setting so that the patient can be monitored. The up-dosing phase is discussed in further detail herein. Following the up-dosing phase, a maintenance dose is administered to the patient, which maintains the patient in a state of desensitization to the food allergen. The duration of the therapy, for example the duration of the up-dosing and/or maintenance phase may vary between patients depending on the age, health conditions, the severity of the peanut allergy, tolerability of the pharmaceutical product, and/or complicating co-morbid diseases or conditions, among other factors. The amount of the doses of the peanut protein composition administered in the up-dosing and maintenance phases can be adjusted as necessary based on the judgment of a patient's medical caregiver and/or the needs of the patient.

A patient undergoing oral immunotherapy for treatment of a food allergy generally has a known or suspected allergy for the food for which treatment is being received. Methods of diagnosing a food allergy are known in the art and include a clinical history of allergic reaction following peanut ingestion, immunological assays (such as measuring peanut-specific IgE (ps-IgE) levels), skin prick tests, and food challenges. For diagnosis of a food allergy by food challenge, the patient generally receives increasing doses of allergenic protein from the food, and an observed allergic reaction to the peanut protein during the food challenge indicates the patient has the food allergy.

A patient undergoing oral immunotherapy for treatment of a peanut allergy may be treatment naïve, having never undergone a peanut oral immunotherapy for the treatment of a food allergy, or may have previously been treated for a food allergy either by oral immunotherapy or by another treatment method. A patient being diagnosed for food allergy by diagnostic exposure to allergenic protein, such as in a food challenge, but with no other history of clinical exposure to allergenic protein, is still considered treatment naïve after the diagnostic exposure.

The patient receiving the oral immunotherapy treatment for the food allergy is a human patient. In some embodiments, the patient is about 1 year of age or older, such as between 1 year of age and about 4 years of age, about 4 years of age and about 12 years of age, between about 12 years of age and about 18 years of age, between about 18 years of age and about 26 years of age, or about 26 years of age or older.

FIG. 2 shows an exemplary oral immunotherapy dosing schedule designed for the treatment of a peanut allergy. The illustrated oral immunotherapy begins with the optional initial escalation phase, which includes oral administration of a series of escalating doses ranging from about 0.5 mg to about 6 mg of peanut protein, which are administered in the same day and spaced by about 20 to about 30 minutes. During this phase, the patient can be monitored for an adverse event or allergenic response under medical supervision, and a medical caregiver can assess the suitability of the oral immunotherapy treatment for the patient. Upon completion of the optional initial escalation phase, the patient begins the up-dosing phase of the treatment. During the up-dosing phase, the subject receives daily scheduled doses of the pharmaceutical composition comprising peanut protein. The same dose is administered on a daily basis for a period of time, before the dose is increased. According to the schedule illustrated in FIG. 2, the patient receives the same dose for two weeks before the dose is escalated. However, that two week period may be longer (for example about 2 weeks to about 4 weeks), or may be shorter (for example, about 1 week to about 2 weeks). The scheduled period of time may be predetermined, or may be adjusted based on the reaction of the patient to the pharmaceutical composition. For example, if the patient experiences no allergic adverse events or only mild allergic adverse events, the period of time may be two weeks before the dose is escalated. On the other hand, if the patient experiences a moderate or severe allergic adverse event, the oral immunotherapy dosing schedule may be adjusted such that the dose escalation is delayed or the dose may be reduced. In addition to allergenic adverse events, the dosing schedule may be adjusted for other concurring factors, such as an infection, inflammation (for example due to surgery, a traumatic injury), menses, or other concurring factors that may lead to heightened sensitivity to the peanut protein. In the example illustrated in FIG. 2, the up-dosing phase includes doses of about 3 mg, about 6 mg, about 12 mg, about 20 mg, about 40 mg, about 80 mg, about 120 mg, about 160 mg, about 200 mg, about 240 mg, and about 300 mg peanut protein, which are administered to the patient in an escalating manner. However, other suitable doses may be used during the up-dosing phase, and (as discussed above), the scheduled dose may be reduced based on the status of the patient. Following the up-dosing phase in the scheduled oral immunotherapy is a maintenance phase, which includes the regular oral administration of a maintenance dose. In some embodiments, the maintenance dose is administered on a daily basis. The maintenance dose may be the same dose as the highest dose reached during the up-dosing phase, but, in some embodiments, it may be higher or lower than the highest dose level. The maintenance phase is administered to the patient to maintain the state of desensitization.

To administer the drug comprising the formulated allergenic material, the composition is orally ingested. Although the pharmaceutical composition may be packaged in an edible dosage form (e.g., a capsule), the pharmaceutical composition is usually removed from the package to allow sufficient oral contact. For example, the pharmaceutical composition may be mixed with a food vehicle, such as oatmeal, pudding, applesauce, yogurt, or other semi-solid food and eaten.

### Initial Escalation Phase

The oral immunotherapy can include an initial dose escalation phase before the up-dosing phase, wherein the patient is administered over the course of one or two days a series of escalating doses. The initial dose escalation phase is distinguished from the up-dosing phase by a lower dose range, shorter intervals between dose escalations, and, typically, closer monitoring by the patient's medical caregiver. For example, a two day initial escalation may comprise a series of doses from about 0.5 mg to about 6 mg peanut protein, such as individual doses of about 0.5 mg, about 1 mg, about 1.5 mg, about 3 mg, and about 6 mg peanut protein. The highest tolerated dose of the initial escalation phase, or a dose lower than the highest tolerated dose in the initial escalation phase, may be the first dose of the up-dosing phase. If a patient does not tolerate at least a certain dose in the initial dose escalation phase, the patient may be excluded from the oral immunotherapy as unsuitable for treatment at that time. For example, if a patient suffers a serious allergic adverse event after administration of the 0.5 mg, 1 mg, or 1.5 mg peanut protein dose, the patient may not be allowed to proceed to the up-dosing phase. The purposes of the initial escalation phase include calibrating the doses of the up-dosing phase (e.g., the initial dose of the up-dosing phase), and ensuring the suitability of the patient for safely entering an up-dosing phase.

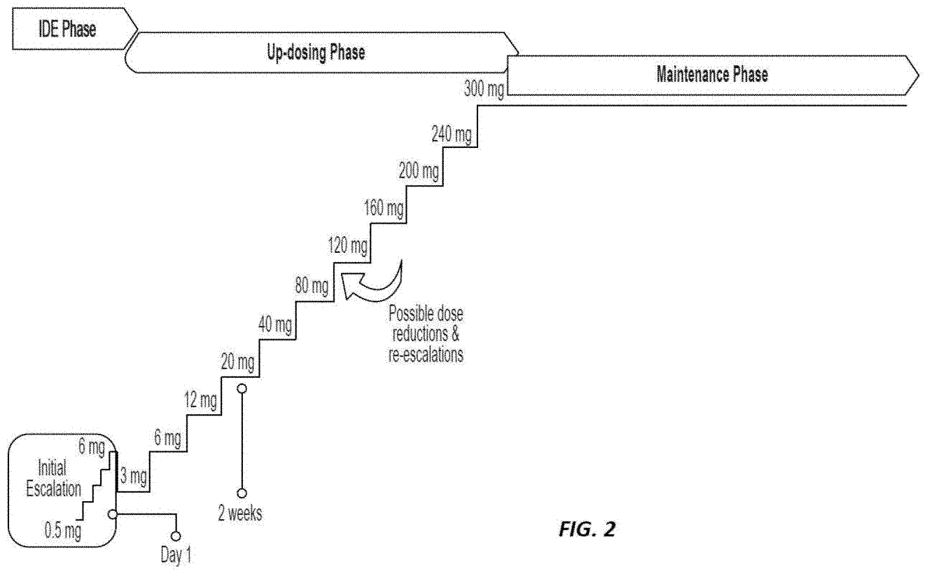

The doses for the initial dose escalation phase can be contained within an initial dose escalation kit. FIG. 3A and 3B illustrate an exemplary initial dose escalation kit that may include doses used in the initial dose escalation phase. The initial dose escalation kit is distributed by a pharmacy enrolled in the drug distribution management database to a healthcare setting enrolled in the drug distribution management database to be used only for the initial dose escalation phase of a particular patient, which must also be enrolled in the drug distribution management database associated with that initial dose escalation kit. Referring to FIG. 3A, and in one embodiment, the initial dose escalation kit 300 can be a single use (non-reusable) bi-fold folio made of cardstock or other material. A front portion 302 of the initial escalation kit 300 can include identifying information 304 that includes, but is not limited to, product and/or company information 306 and space 308 for pharmacy prescription label and/or patient information (e.g., name of the patient, or other patient identification). FIG. 3B is a front view of the initial dose escalation kit 300 of FIG. 3A in an open configuration. As illustrated, the initial dose escalation kit 300 comprises content information 310 on a first side 312 of an internal portion 314 of the kit 300. The content information 310 may include, for example, dose preparation instructions 316, dosage administration instructions 318, storage instructions 320, ingredients 322 and company/product identification information 324 among other information. A second side 326 of the internal portion 314 comprises a series of dose units 328 from a first lowest dose 330 to a maximum dose 332 that are releasably retained within the folio material. For example, the dose units 328 may be retained within individual foil packs attached to or integrated with the second side 326 of the kit 300. During an initial escalation phase, a patient can be prescribed an initial escalation kit 300 wherein each of the dose units 328 are administered to the patient within a healthcare setting in pre-determined or physician-determined time intervals (e.g., according to an OIT regimen). For example, during the course of an initial escalation phase, the patient can be administered the first lowest dose 330 up to the maximum dose 332 as tolerated.

The initial escalation phase is administered under medical supervision (such as under the supervision of a healthcare provider enrolled in the drug distribution management database) in a healthcare setting enrolled in the drug distribution management database. The patient is usually closely monitored by a medical caregiver (such as a healthcare provider), who can provide interventions such as epinephrine, albuterol, and diphenhydramine in the event of an allergic adverse reaction that necessitates intervention. The initial escalation phase of the oral immunotherapy, if present, includes administration of a plurality of small doses of the peanut protein composition to the patient. The small doses can be spaced by a period of time, such as about 10 minutes to about 60 minutes, and can include 1, 2, 3, 4, or 5 or more doses.

In one example, the initial escalation phase comprises doses between about 0.5 mg and about 6 mg peanut protein, such as about 0.5 mg to about 1.5 mg peanut protein, about 1.5 mg to about 3 mg peanut protein, or about 3 mg to about 6 mg peanut protein. In a non-limiting example, the initial escalation phase comprises an incremental escalation over one day from about 0.5 mg peanut protein to a maximum of about 6 mg peanut protein in a single day, with single doses of about 0.5 mg, about 1 mg, about 1.5 mg, about 3 mg, and about 6 mg of peanut protein, wherein tolerance of the 3 mg or 6 mg peanut protein dose indicates the patient can safely proceed to an up-dosing phase of an oral immunotherapy.

### Up-Dosing Phase

The up-dosing phase includes orally administering to the patient a series of escalating doses of the pharmaceutical composition on a daily basis, wherein a given dose is administered to the patient for at least a predetermined period of time before the dose is escalated to reach the maximum dose administered to the patient during the course of the oral immunotherapy. The length of time of the up-dosing phase can be adapted according the needs of an individual patient, although is generally completed in about 22 to about 40 weeks. For some patients, the up-dosing phase may last as long as 2 years or more. The up-dosing phase may be extended, for example, if a patient experiences allergic adverse events after beginning a higher dose in the dosing series.

The pharmaceutical composition administered to the patient during the up-dosing phase is administered at pre-set escalating dose levels. The patient slowly escalates the dose levels under medical direction (such as under the direction of a healthcare provider) until a maximum dose is reached, generally the same dose as the maintenance dose. The same dose level (i.e., the same dose of peanut protein) is administered to the patient for a period of time as the patient becomes desensitized to that amount before the dose level is increased. In some embodiments, the up-dosing phase comprises a series of escalating doses of the pharmaceutical composition at about 5 or more different dose levels (for example 5 to 15 different dose levels, such as 5, 6, 7, 8, 9, 10, 11, 12, 13, 14, or 15 different dose levels). In a non-limiting preferred embodiment, the up-dosing phase includes 11 different dose levels. For example, the up-dosing phase may include dose levels of about 3 mg, about 6 mg, about 12 mg, about 20 mg, about 40 mg, about 80 mg, about 120 mg, about 160 mg, about 200 mg, about 240 mg, and about 300 mg peanut protein.

The up-dosing phase of a peanut OIT typically involves incrementally increasing the administered peanut protein dose after a period of time (e.g., approximately every 1-4 weeks). A particular dose in the series is repeatedly (e.g., daily) administered to the patient until advancing to the next dose in the series. In some instances, such as when the patient does not tolerate a particular dose in the series or the patient experiences one or more allergic adverse events, the dose is decreased or the dose in the series is repeated for a period of time prior to advancing to the next dose in the series. The rate of up-dosing (e.g., the length of time an individual dose in the series is administered or the size of the dose increment between doses in the series) may be adjusted based on one or more observed allergic adverse events.

The doses administered to the patient during the up-dosing phase are generally administered on a daily basis for a period of time, such as about 1 week to about 4 weeks, such as about 2 weeks. After the completion of a particular dose in the series for a period of time, treatment can be advanced to a higher dose in the series. In some embodiments, the up-dosing phase of the treatment comprises a series of between 5 and 15 different dose levels. If a patient tolerates a particular dose level during the up-dosing phase for a period of time, the patient can advance to the next dose level in the series of the up-dosing phase. If a patient does not tolerate a particular dose level during the up-dosing phase for a period of time, the patient may repeat the current dose level in the series. Alternatively, if a patient does not tolerate a particular dose level during the up-dosing phase for a period of time, the patient may return to an earlier dose level in the series. The duration of the up-dosing phase therefore depends on the specific responses of the patient. The patient may repeat doses in the series as many times as necessary to achieve the highest dose in the series. The up-dosing phase ends when the highest dose is tolerated for two weeks.

The dose levels of the drug for oral immunotherapy used to treat peanut allergy, for example, are administered during the up-dosing phase are generally between about 0.5 mg and about 1000 mg of peanut protein, such as about 0.5 mg to about 10 mg peanut protein, about 10 mg to about 100 mg peanut protein, about 100 mg to about 300 mg peanut protein, about 300 mg to about 500 mg peanut protein, or about 500 mg to about 1000 mg peanut protein. In a non-limiting exemplary embodiment, the dose levels of the up-dosing phase include doses of about 3 mg peanut protein, about 6 mg peanut protein, about 12 mg peanut protein, about 20 mg peanut protein, about 40 mg peanut protein, about 80 mg peanut protein, about 120 mg peanut protein, about 160 mg peanut protein, about 200 mg peanut protein, about 240 mg peanut protein, and about 300 mg peanut protein, wherein each dose level is administered on a daily basis for about 1 week to about 4 weeks (such as about 2 weeks) before escalating to the next highest dose.

The doses administered to the patient during the up-dosing phase are adjusted periodically, such as between once every week and once every six weeks. In some embodiments, the up-dosing phase comprises weekly dose adjustment, dose adjustment every two weeks, dose adjustment every third week, dose adjust every fourth week, dose adjustment every fifth week, dose adjustment every sixth week, or adjustment as needed based on the judgment of the patient's medical caregiver. The dose may be increased to the next scheduled dose in the series, lowered to a previous in the series in response to an allergic adverse event, maintained for an additional period of time at the current dose in the series, increased to a higher dose in the series based on the judgment of the patient's medical caregiver, or decreased to a lower dose in the series based on the judgment of the patient's medical caregiver. In some embodiments, the up-dosing phase is adjusted at any time based on the judgment of the patient's medical caregiver that the patient did not tolerate the current dose in the series.

The first dose of any dose level is administered to the patient in a healthcare setting enrolled in the drug distribution management database. The healthcare setting has possession of an oral immunotherapy treatment kit (which may be distributed to the healthcare setting by a distributor enrolled in the drug distribution management database after verifying the healthcare setting is enrolled in the drug distribution management database) that includes a plurality of doses at different dose levels. The healthcare provider can retrieve a dose of the drug at the prescribed dose level for administration to the patient in the healthcare setting. Once the dose has been administered to the patient, the patient is monitored within the healthcare setting for an adverse reaction. After monitoring the patient, the healthcare provider can prescribe to the patient additional doses of the drug at that dose level, and those additional doses at the prescribed dose level can be distributed to the patient or the healthcare setting by a pharmacy enrolled in the drug distribution management database. In some embodiments, the prescription for that dose level is provided only if the patient does not experience a clinically significant allergic response to the drug. In some embodiments, the prescription for that dose level is provided only if the patient does not experience a moderate or severe allergic response to the drug. In some embodiments, the prescription for that dose level is provided only if the patient does not experience a severe allergic response to the drug. In some embodiments, the prescription for that dose level is provided only if the patient does not experience a systemic allergic response to the drug. The patient (or parent, guardian, or other healthcare provider of the patient) may then self-medicate at the prescribed dose level for the prescribed period of time and until the patient returns to the healthcare setting. Based on the patient's tolerance of the dose level, the healthcare provider may then administer, in the healthcare setting, the drug at a higher dose level, a lower dose level, or the same dose level before monitoring the patient for an allergic response in the healthcare setting.

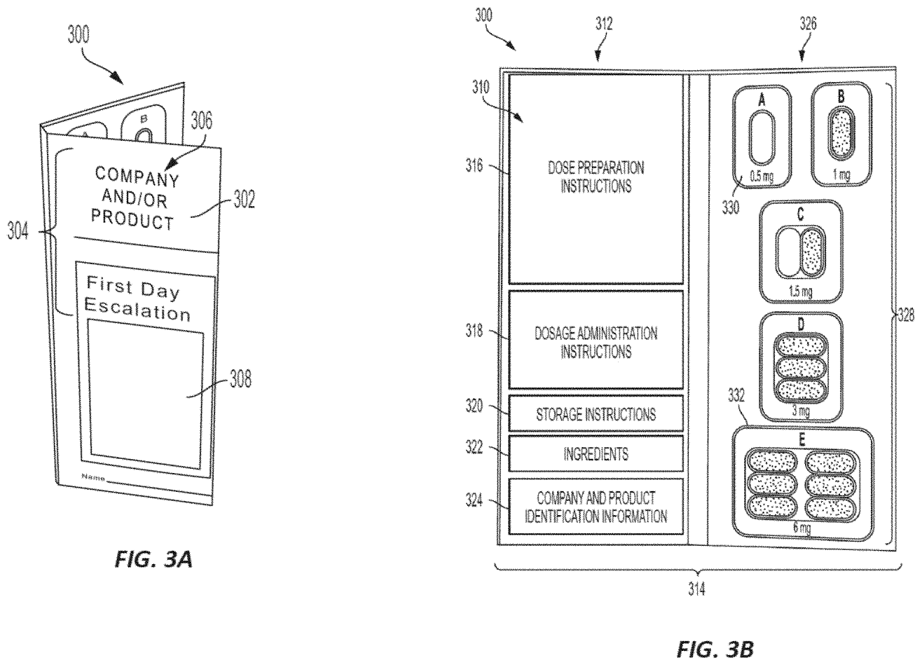

FIG. 4 shows an exemplary oral immunotherapy treatment kit that may be in the possession of a healthcare setting enrolled in the drug distribution management database. The oral immunotherapy treatment kit 400, for example, may be used to store, organize, identify and/or select a proper dosage level during OIT administration. The illustrated oral immunotherapy treatment kit 400 includes a housing 402 having a base portion 412 with an internal cavity 414 that retains a plurality of dose containers 420a and 420b. Each dose container holds a plurality of single dose packages 430a and 430b. The single dose packages 430a and 430b each contain a single dose of the drug at the indicate dose level. The single dose packages within a given dosage container contain identical dosages. For example, dosage container 420a contains a plurality of single dose packages 430a with each single dose package 430a providing a first dose level (e.g., 3 mg), and dosage container 420b contains a plurality of single dose packages 430b with each single dose package 430b providing a second dose level (e.g., 6 mg), wherein the first dose level and the second dose level are different. During normal use, the unit dose dispensing system 400 comprising the housing 402, dosage containers 420 and single dose packages 430 are maintained at a physician's office or medical clinic, and facilitates efficiency and dosage accuracy during in-office patient OIT treatment.

The up-dosing phase proceeds until the patient achieves the final dose in the up-dosing series. In some embodiments, the up-dosing phase is about 1 month to about 6 months, such as about 1 month to about 3 months, or about 3 months to about 6 months. In some embodiments, the up-dosing phase is about 6 months to about 2 years, such as about 6 months to about 1 year, about 1 year to about 18 months, or about 18 months to about 2 years. In a non-limiting exemplary embodiment, the up-dosing phase continues for 22 weeks to 2 years, depending on the number of dose reductions and re-escalations and dose level repeats. In any of the described embodiments, the up-dosing phase terminates when the patient tolerates the scheduled dose of the final dose in the series of the up-dosing phase for 2 weeks, thereby beginning the maintenance phase.

Each dose of the series of the up-dosing phase may be scheduled to last about 1 week, about 2 weeks, about 3 weeks, about 4 weeks, or values and ranges therebetween. Based on the observation of an allergic adverse event, a patient's caregiver may repeat the patient's current dose in up-dosing series. A particular portion with a particular dose may be repeated as many times as necessary, such as once, two times, three times, or four times, or more, to adequately desensitize a patient to that dose, such as when the patient no longer experiences a moderate or serious allergic adverse event upon accidental (or deliberate) exposure to the food allergen.

### Maintenance Phase

The maintenance phase of the oral immunotherapy begins after the termination of the up-dosing phase. In any of the methods described herein, the maintenance phase comprises orally administering to the patient a plurality of maintenance doses of the pharmaceutical composition. The maintenance phase may be initiated in the healthcare setting by administering to the patient a drug at the maintenance dose level (which may be contained, for example, in the oral immunotherapy treatment kit possessed by the healthcare setting). After administering the maintenance dose to the patient, the patient is monitored for an allergic response before the healthcare provider prescribes the maintenance dose level to the patient. A pharmacy enrolled in the drug distribution management database can then distribute the a drug doses at the maintenance dose level to the patient enrolled in the drug distribution management database or a healthcare setting enrolled in the drug distribution management database.

The duration of the maintenance phase is generally about 20 weeks or longer, and may be for the entire life of the patient. For example, in some embodiments, the maintenance phase is about 20 weeks to about 24 weeks, about 24 weeks to about 28 weeks, about 28 weeks to about 32 weeks, about 32 weeks to about 36 weeks, about 36 weeks to about 40 weeks, about 40 weeks to about 44 weeks, about 44 weeks to about 48 weeks, about 48 weeks to about 52 weeks, about 52 weeks to about 60 weeks, about 60 weeks to about 72 weeks, about 72 weeks to about 80 weeks, or more than about 80 weeks, such as the life of the patient.

The dose of peanut protein administered to the patient during the maintenance phase is between about 200 mg and about 1000 mg peanut protein, unless the maintenance phase dose has been reduced for example according to the methods described herein when the patient misses one or more consecutive doses. For example, in some embodiments, a dose during the maintenance phase is between about 200 mg and about 300 mg peanut protein, about 300 mg and about 500 mg peanut protein, about 500 mg and about 1000 mg peanut protein, or values and ranges therebetween, unless the maintenance phase dose has been reduced for example according to the methods described herein when the patient misses one or more consecutive doses. The scheduled maintenance phase dose can be at the same dose or a higher dose as the dose administered during the final sub-phase of the up-dosing phase. In a non-limiting exemplary embodiment, a maintenance phase dose administered to the patient during the maintenance phase is about 300 mg peanut protein. In some embodiments, the maintenance phase dose is administered daily.

### Drug Compositions

The drug used to treat the food allergy is specific to the food allergy being treated, and includes food allergens (such as allergenic proteins) that are ordinarily found in that food. For example, a drug used to treat a peanut allergy can include allergenic peanut proteins (e.g., Ara h 1, Ara h2, Ara h 6, etc.). In some embodiments, the drug used to treat peanut allergy comprises peanut flour formulated with one or more excipients. In another example, a drug used to treat an egg allergy includes allergenic egg proteins (e.g., ovalbumin, ovomucoid, lysozyme, etc.). In some embodiments, the drug used to treat egg allergy comprises dried egg white protein powder formulated with one or more excipients.

The drug may be contained within an openable package (such as a sachet, or an openable capsule (or “sprinkle capsule”)) in an amount in accordance with the dose level for the individual package. The package may be opened and the drug contents of the package are poured into a semi-solid food (e.g., yogurt, applesauce, oatmeal, pudding, etc.) and mixed. The food mixed with the drug is then consumed by the patient. In some embodiments, the drug is formulated as a free-flowing powder, which allows the drug to be easily poured from the package.

Exemplary drug compositions are described in U.S. Pat. No. 9,198,869; U.S. Pat. No. 9,492,535; U.S. Pat. No. 10,086,068; and U.S. Pat. No. 10,449,118; WO 2016/033094; and WO 2018/132733, each of which is incorporated herein by reference for all purposes.

In some embodiments, the drug composition is peanut (Arachis hypogaea) allergen powder.

In some embodiments, the invention provides a drug comprising a food allergen for use in treating a patient for a food allergy by oral immunotherapy, wherein the drug is administered or provided to the patient according to any of the methods as described herein.

### Treatment of a Food Allergy

The drug used to treat food allergy contains allergenic proteins that induce an immune response in a patient allergic to the food. The immediate induced immune response is generally mild or not clinically apparent, although over time the patient become desensitized to the allergenic proteins due to a change in the immune system during the course of treatment. Occasionally, however, a clinically significant (and even severe or systemic) allergic response may occur after treating the patient with the drug. Thus, the distribution and administration controls described herein are used to minimize the risk to the patient.

Prior to treatment, the patient must be enrolled in the drug distribution management database. In some embodiments, the healthcare setting overseeing treatment of the patient must enroll the patient in the drug distribution management database or otherwise verify enrollment of the patient in the drug distribution management database for the drug used to treat the food allergy. In some embodiments, the prescriber (such as the healthcare provider) overseeing treatment of the patient enrolls the patient in the drug distribution management database or otherwise verify enrollment of the patient in the drug distribution management database for the drug used to treat the food allergy. Once the patient is enrolled in (or enrollment is otherwise verified) the drug distribution management database, the healthcare setting (which must also be enrolled in the drug distribution management database) and/or the healthcare provider (who must also be enrolled in the drug distribution management database) submits a request for an initial dose escalation kit. The initial dose escalation kit is received by the healthcare setting from a pharmacy, which must also be enrolled in the drug distribution management database. In some embodiments, the pharmacy must verify enrollment of the patient and the healthcare setting in the drug distribution management database before providing the initial dose escalation kit to the healthcare setting. In some embodiments, the pharmacy verifies enrollment of the patient and the healthcare provider in the drug distribution management database before providing the initial dose escalation kit to the healthcare setting. In some embodiments, the pharmacy verifies enrollment of the patient, the healthcare provider, and the healthcare setting in the drug distribution management database before providing the initial dose escalation kit to the healthcare setting. The initial dose escalation kit is specific for the patient being treated. The initial dose escalation kit includes a plurality of escalating doses, which are administered to the patient in the healthcare setting. The doses are administered to the patient one at a time, and the patient is monitored in the healthcare setting for an allergic response during a monitoring period.

After initiating treatment using the initial dose escalation kit, the up-dosing phase may begin. The drug is administered to the patient at an initial dose level, and the dose level is increased periodically depending on patient response. Although the general trajectory during the up-dosing phase is upwards, there may be times when the healthcare provider treats the patient with a lower dose level or the same dose level after the dosing period for a level is complete.

The first dose of the drug at any dose level is administered to the patient in the healthcare setting, and the patient is monitored, in the healthcare setting, for an allergic response to the drug. If the patient tolerates the drug at that dose level, the treating healthcare provider can prescribe a plurality of doses at that dose level to the patient. The plurality of doses at that drug dose level can be obtained from a pharmacy, which may be enrolled in the drug distribution management database. In some embodiments, the pharmacy must verify enrollment of the patient and the healthcare setting in the drug distribution management database. In some embodiments, the pharmacy verifies enrollment of the patient and the healthcare provider in the drug distribution management database. In some embodiments, the pharmacy verifies enrollment of the patient, the healthcare provider, and the healthcare setting in the drug distribution management database. After the plurality of doses of the drug are administered to the patient, the patient can return to the healthcare setting. At the healthcare setting, a dose at the subsequent dose level is administered to the patient, and the patient is monitored for an allergic reaction to the drug. The healthcare setting (and/or the healthcare provider) may then provide a subsequent prescription for the subsequent level of the drug, which can be obtained from a pharmacy enrolled in the drug distribution management database after the pharmacy verifies, in various embodiments, that the healthcare setting and the patient; the healthcare provider and the patient; or the healthcare setting, the healthcare provider, and the patient are enrolled in the drug distribution management database. In some embodiments, the prescription for that dose level is provided only if the patient does not experience a clinically significant allergic response to the drug. In some embodiments, the prescription for that dose level is provided only if the patient does not experience a moderate or severe allergic response to the drug. In some embodiments, the prescription for that dose level is provided only if the patient does not experience a severe allergic response to the drug. In some embodiments, the prescription for that dose level is provided only if the patient does not experience a systemic allergic response to the drug. This process is repeated through the up-dosing phase and through the maintenance level dosing.

In some embodiments, a method of treating a patient for a food allergy by oral immunotherapy includes (a) enrolling the patient or verifying enrollment of the patient in a drug distribution management database for a drug used to treat the food allergy; (b) administering to the enrolled patient, in a healthcare setting enrolled in the drug distribution management database, a dose of the drug at a first up-dosing level according to an up-dosing schedule; (c) monitoring the enrolled patient, in the enrolled healthcare setting, for an allergic response to the drug after administering the dose of the drug in step (b); and (d) providing a first prescription requesting a pharmacy enrolled in the drug distribution management database release to the enrolled patient or the enrolled healthcare setting a plurality of doses of the drug at the first up-dosing level. In some embodiments, the administration of a dose of the drug at a first up-dosing level according to an up-dosing schedule is under the supervision of a healthcare provider enrolled in the drug distribution management database. In some embodiments, monitoring the enrolled patient is under the supervision of a healthcare provider enrolled in the drug distribution management database.

In some embodiments, a method of treating a patient for a food allergy by oral immunotherapy includes (a) enrolling the patient or verifying enrollment of the patient in a drug distribution management database for a drug used to treat the food allergy; (b) administering to the enrolled patient, in a healthcare setting enrolled in the drug distribution management database, a dose of the drug at a first up-dosing level according to an up-dosing schedule; (c) monitoring the enrolled patient, in the enrolled healthcare setting, for an allergic response to the drug after administering the dose of the drug in step (b); (d) providing a first prescription requesting a pharmacy enrolled in the drug distribution management database release to the enrolled patient or the enrolled healthcare setting a plurality of doses of the drug at the first up-dosing level; (e) administering to the enrolled patient, in the enrolled healthcare setting, a dose of the drug at a subsequent up-dosing level according to the up-dosing schedule; (f) monitoring the enrolled patient, in the enrolled healthcare setting, for an allergic response to the drug after administering the dose of the drug in step (e); and (g) providing a subsequent prescription requesting the enrolled pharmacy release to the enrolled patient or the enrolled healthcare setting a plurality of doses of the drug at the subsequent up-dosing level. In some embodiments, administering to the enrolled patient the dose of the drug at a first up-dosing level according to an up-dosing schedule is under the supervision of a healthcare provider enrolled in the drug distribution management database. In some embodiments, administering to the enrolled patient a dose of the drug at a subsequent up-dosing level according to the up-dosing schedule is under the supervision of a healthcare provider enrolled in the drug distribution management database. In some embodiments, the first prescription and/or the subsequent prescription is provided by a healthcare provider enrolled in the drug distribution management database.

In some embodiments, a method of treating a patient for a food allergy by oral immunotherapy includes (a) enrolling the patient or verifying enrollment of the patient in a drug distribution management database for a drug used to treat the food allergy; (b) administering to the enrolled patient, in a healthcare setting enrolled in the drug distribution management database, a plurality of doses of the drug from an initial dose escalation kit; (c) monitoring the enrolled patient, in the enrolled healthcare setting, for an allergic response to the drug after administering the each dose of the drug in step (b); (d)) administering to the enrolled patient, in a healthcare setting enrolled in the drug distribution management database, a dose of the drug at a first up-dosing level according to an up-dosing schedule; (e) monitoring the enrolled patient, in the enrolled healthcare setting, for an allergic response to the drug after administering the dose of the drug in step (d); (f) providing a first prescription requesting a pharmacy enrolled in the drug distribution management database release to the enrolled patient or the enrolled healthcare setting a plurality of doses of the drug at the first up-dosing level; (g) administering to the enrolled patient, in the enrolled healthcare setting, a dose of the drug at a subsequent up-dosing level according to the up-dosing schedule; (h) monitoring the enrolled patient, in the enrolled healthcare setting, for an allergic response to the drug after administering the dose of the drug in step (g); and (i) providing a subsequent prescription requesting the enrolled pharmacy release to the enrolled patient or the enrolled healthcare setting a plurality of doses of the drug at the subsequent up-dosing level. In some embodiments, administering to the enrolled patient the plurality of doses of the drug from an initial dose escalation kit is under the supervision of a healthcare provider enrolled in the drug distribution management database. In some embodiments, administering to the enrolled patient a dose of the drug at a first up-dosing level according to an up-dosing schedule is under the supervision of a healthcare provider enrolled in the drug distribution management database. In some embodiments, a healthcare provider enrolled in the drug distribution management database provides the first prescription. In some embodiments, administering to the enrolled patient a dose of the drug at a subsequent up-dosing level according to the up-dosing schedule is under the supervision of a healthcare provider enrolled in the drug distribution management database. In some embodiments, the subsequent prescription is provided by a healthcare provider enrolled in the drug distribution management database.

In some embodiments, a method of treating a patient for a food allergy by oral immunotherapy includes (a) enrolling the patient or verifying enrollment of the patient in a drug distribution management database for a drug used to treat the food allergy; (b) administering to the enrolled patient a dose of the drug at a first up-dosing level according to an up-dosing schedule, wherein the dose is administered under the supervision of a healthcare provider enrolled in the drug distribution management database; (c) monitoring the enrolled patient, under the supervision of the enrolled healthcare provider, for an allergic response to the drug after administering the dose of the drug in step (b); (d) providing a first prescription requesting a pharmacy enrolled in the drug distribution management database release to the enrolled patient, the enrolled healthcare provider, or an enrolled healthcare setting a plurality of doses of the drug at the first up-dosing level; (e) administering to the enrolled patient a dose of the drug at a subsequent up-dosing level according to the up-dosing schedule, wherein the dose is administered under the supervision of the healthcare provider; (f) monitoring the enrolled patient for an allergic response to the drug after administering the dose of the drug in step (e); and (g) providing a subsequent prescription requesting the enrolled pharmacy release to the enrolled patient, the enrolled healthcare provider, or a healthcare setting enrolled in the drug distribution management database a plurality of doses of the drug at the subsequent up-dosing level.

### Drug Distribution

As discussed herein, the distribution and administration of the drug is tightly controlled to minimize risk to the patient and to ensure the patient receives the correct amount of the drug at the proper phase of treatment. These controls include controls over the distribution and/or administration of the drug by the various involved entities such as the distributor, the pharmacy, the healthcare setting, and the patient.

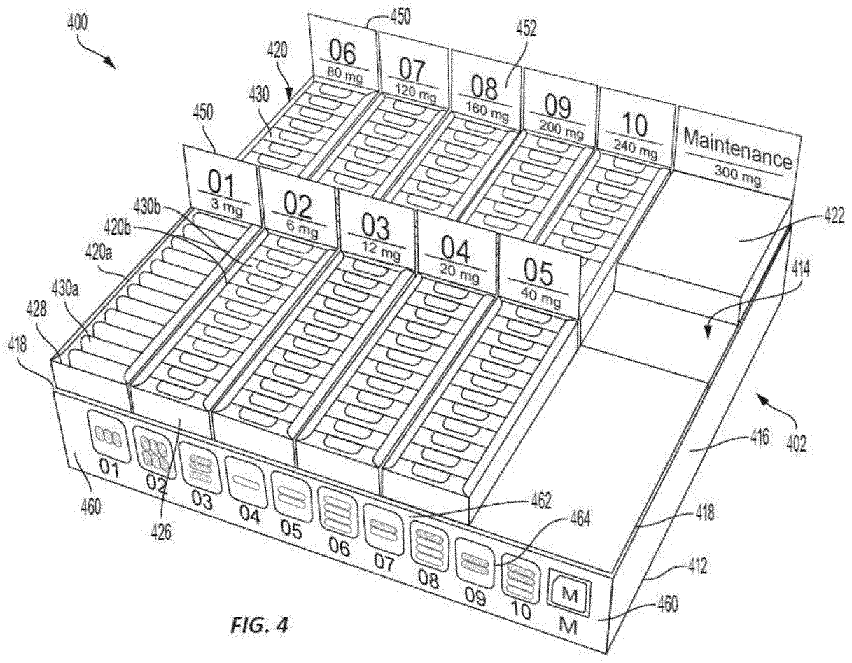

FIG. 5 provides an overview of exemplary distribution controls for the drug distribution methods described herein. FIG. 5 shows the roles of the prescriber 501, the patient (or parent or guardian of the patient) 502, the healthcare setting 503, the pharmacy 504, the distributor 505, and the drug distribution management database manager 506. The prescriber 501 manages the treatment of the patient 502 for the food allergy, and is preferably associated with the healthcare setting 503. An electronic system 507 (which includes a memory that stores the drug distribution management database 508 and one or more processors 509 configured to access the memory and execute one or more programs that accesses the information in the drug distribution management database 508) is used to manage authorization for drug distribution. The drug distribution management database 508 includes an enrolled patient sub-database 510, an enrolled healthcare setting sub-database 511, an enrolled pharmacy sub-database 512, and an enrolled distributor sub-database 513. The prescriber 501 provides a prescription or request 514 that the pharmacy 504 provide an initial dose escalation kit to a healthcare setting 503 for a specific patient 502. The initial dose escalation kit is specific to the patient 502, and should not be used to initiate treatment for a different patient. At 515, the pharmacy receives the prescription or request 514, and verifies that the patient 502 is enrolled in the patient sub-database 510 and that the healthcare setting 503 is enrolled in the healthcare setting sub-database 511 by sending a verification request to the drug distribution management database manager 506. In some embodiments, verification occurs automatically; for example, the verification request may be received by a computer system communicatively connected to the drug distribution management database, which is automatically accessed to compare the information in the verification request to the information in the database. If a match between the information in the verification request and the information in the database is determined, an enrollment status may be automatically generated. In some embodiments, the enrollment status is automatically transmitted to the pharmacy in response to receiving the request. Once a positive enrollment verification has been received by the pharmacy 504, the pharmacy 504 provides the patient-specific initial dose escalation kit to the healthcare setting 503 (as indicated at 516). Before or after receipt of the initial dose escalation kit, but prior to initiating treatment of the patient, the healthcare setting 503 verifies enrollment of the patient in the patient sub-database 510, at step 517. Verification of patient 502 enrollment in the patient sub-database can be performed by reviewing records that a patient enrollment request has been transmitted to the drug distribution management database manager 506 and that an enrollment confirmation has been received from the drug distribution management database manager 506, or by sending an enrollment verification request to the drug distribution management database manager 506 of the patient 502. Upon receipt of the enrollment verification request, the drug distribution management database manager 506 can compare patient information in the request to patient information in the patient sub-database 510 to verify enrollment of the patient 502, and the drug distribution management database manager can then transmit a positive enrollment verification for the patient 502 to the healthcare setting 503. In some embodiments, verification occurs automatically; for example, the verification request may be received by a computer system communicatively connected to the drug distribution management database, which is automatically accessed to compare the information in the verification request to the information in the database. If a match between the information in the verification request and the information in the database is determined, an enrollment status may be automatically generated. In some embodiments, the enrollment status is automatically transmitted to the healthcare setting in response to receiving the request. Once the healthcare setting 503 has confirmed enrollment of the patient 502 in the patient sub-database 510, the drug can be administered to the patient 502 from the initial dose escalation kit in accordance with an initial dose escalation schedule, at step 518.

Food allergy treatment using the drug by oral immunotherapy during the up-dosing phase includes two sub-steps at each dosing level. The first sub-step includes administration of a first dose of the drug at the selected dosing level to the patient 502 in the healthcare setting 503. The second sub-step includes administration of a plurality of doses (for example, one dose per day) over a period of time of the drug at the same dose level, which the patient 502 may receive from a pharmacy 504 and administer in an unsupervised setting (i.e., at a location other than in the healthcare setting). The healthcare setting 503 can retrieve the first dose of each dosing level for administration to the patient 502 from an oral immunotherapy treatment kit. At step 519, a prescriber 501 requests, from a distributor 505, the oral immunotherapy treatment kit be provided to the healthcare setting 503. The distributor 505 transmits an enrollment verification request containing healthcare setting information to the drug distribution management database manager. The drug distribution management database manager compares the information contained within the enrollment verification request to healthcare setting information in the healthcare setting sub-database 511. If the healthcare setting 503 is enrolled in the healthcare setting sub-database, the drug distribution management database manager 506 can transmit a positive enrollment verification to the distributor 505, which authorizes the distributor to provide the oral immunotherapy treatment kit to the healthcare setting 503, as indicated at 521. In some embodiments, verification occurs automatically; for example, the verification request may be received by a computer system communicatively connected to the drug distribution management database, which is automatically accessed to compare the information in the verification request to the information in the database. If a match between the information in the verification request and the information in the database is determined, an enrollment status may be automatically generated. In some embodiments, the enrollment status is automatically transmitted to the healthcare setting in response to receiving the request. The healthcare setting 503 can then use doses of the drug in the oral immunotherapy treatment kit to administer the first dose of the drug at the selected level to the patient 502, as shown at 522. The patient 502 is monitored at the healthcare setting 503 before the prescriber 501 provides a prescription or request 523 for a pharmacy 504 to provide a plurality of doses of the drug at the same dose level for the patient 502. The pharmacy 504 receives the prescription or request 523, and may provide the plurality of doses of the drug to the healthcare setting 503 (at 524) or the patient 502 (at 525) only after verifying enrollment of the healthcare setting 503 in the healthcare setting sub-database 511 and the patient 502 in the patient sub-database 510, as indicated at 526. The pharmacy can submit a healthcare setting enrollment request containing healthcare setting information and a patient enrollment verification request containing patient information to the drug distribution management database manager 506. The drug distribution management database manager 506 can compare the received information to the information in the drug distribution management database 508, and can provide an enrollment verification to the pharmacy 504, which authorizes the pharmacy 504 to provide the drug to the patient 502 or healthcare setting 503. In some embodiments, verification occurs automatically; for example, the verification request may be received by a computer system communicatively connected to the drug distribution management database, which is automatically accessed to compare the information in the verification request to the information in the database. If a match between the information in the verification request and the information in the database is determined, an enrollment status may be automatically generated. In some embodiments, the enrollment status is automatically transmitted to the pharmacy in response to receiving the request. The pharmacy 504 should only provide a single dose level of the drug for any given patient 502 at a time.

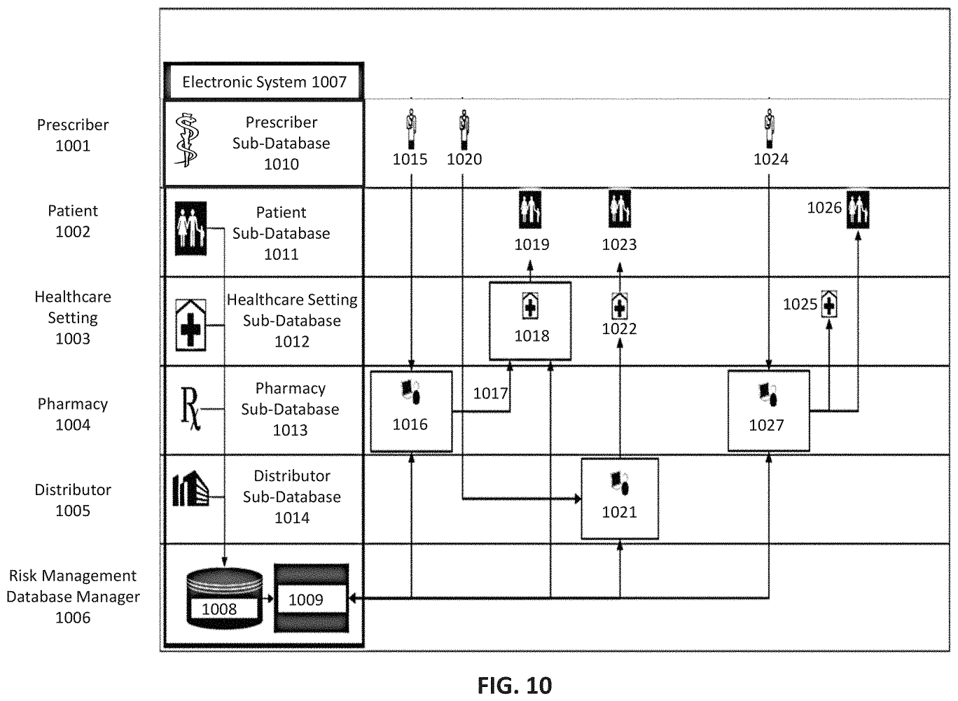

FIG. 10 provides an additional overview of exemplary distribution controls for the drug distribution methods described herein. FIG. 10 shows the roles of the prescriber (also termed synonymously herein as the healthcare provider) 1001, the patient (or parent or guardian of the patient) 1002, the healthcare setting 1003, the pharmacy 1004, the distributor 1005, and the drug distribution management database manager 1006. The prescriber 1001 manages the treatment of the patient 1002 for the food allergy, and is preferably associated with the healthcare setting 1003. An electronic system 1007 (which includes a memory that stores the drug distribution management database 1008 and one or more processors 1009 configured to access the memory and execute one or more programs that accesses the information in the drug distribution management database 1008) is used to manage authorization for drug distribution. The drug distribution management database 1008 includes an enrolled prescriber sub-database 1010, an enrolled patient sub-database 1011, an enrolled healthcare setting sub-database 1012, an enrolled pharmacy sub-database 1013, and an enrolled distributor sub-database 1014. The prescriber 1001 provides a prescription or request 1015 that the pharmacy 1004 provide an initial dose escalation kit to a healthcare setting 1003 for a specific patient 1002. The initial dose escalation kit is specific to the patient 1002, and should not be used to initiate treatment for a different patient. At 1016, the pharmacy receives the prescription or request 1015, and verifies that the patient 1002 is enrolled in the patient sub-database 1011, that the prescriber 1001 is enrolled in the prescriber sub-database 1010, and, in some embodiments, that the healthcare setting 1003 is enrolled in the healthcare setting sub-database 1012 by sending a verification request to the drug distribution management database manager 1006. In some embodiments, verification occurs automatically; for example, the verification request may be received by a computer system communicatively connected to the drug distribution management database, which is automatically accessed to compare the information in the verification request to the information in the database. If a match between the information in the verification request and the information in the database is determined, an enrollment status may be automatically generated. In some embodiments, the enrollment status is automatically transmitted to the pharmacy in response to receiving the request. Once a positive enrollment verification has been received by the pharmacy 1004, the pharmacy 1004 provides the patient-specific initial dose escalation kit to the healthcare setting 1003 (as indicated at 1017). Before or after receipt of the initial dose escalation kit, but prior to initiating treatment of the patient, the healthcare setting 1003 verifies enrollment of the patient in the patient sub-database 1011, at step 1018. Verification of patient 1002 enrollment in the patient sub-database can be performed by reviewing records that a patient enrollment request has been transmitted to the drug distribution management database manager 1006 and that an enrollment confirmation has been received from the drug distribution management database manager 1006, or by sending an enrollment verification request to the drug distribution management database manager 1006 of the patient 1002. Upon receipt of the enrollment verification request, the drug distribution management database manager 1006 can compare patient information in the request to patient information in the patient sub-database 1011 to verify enrollment of the patient 1002, and the drug distribution management database manager can then transmit a positive enrollment verification for the patient 1002 to the healthcare setting 1003. In some embodiments, verification occurs automatically; for example, the verification request may be received by a computer system communicatively connected to the drug distribution management database, which is automatically accessed to compare the information in the verification request to the information in the database. If a match between the information in the verification request and the information in the database is determined, an enrollment status may be automatically generated. In some embodiments, the enrollment status is automatically transmitted to the healthcare setting in response to receiving the request. Once the healthcare setting 1003 has confirmed enrollment of the patient 1002 in the patient sub-database 1011, the drug can be administered to the patient 1002 from the initial dose escalation kit in accordance with an initial dose escalation schedule, at step 1019.

Food allergy treatment using the drug by oral immunotherapy during the up-dosing phase includes two sub-steps at each dosing level. The first sub-step includes administration of a first dose of the drug at the selected dosing level to the patient 1002 in the healthcare setting 1003 by the prescriber 1001. The second sub-step includes administration of a plurality of doses (for example, one dose per day) over a period of time of the drug at the same dose level, which the patient 1002 may receive from a pharmacy 1004 and administer in an unsupervised setting (i.e., at a location other than in the healthcare setting). The healthcare setting 1003 can retrieve the first dose of each dosing level for administration to the patient 1002 from an oral immunotherapy treatment kit. At step 1020, a prescriber 1001 requests, from a distributor 1005, the oral immunotherapy treatment kit be provided to the healthcare setting 1003. The distributor 1005 at step 1021 receives the request 1020 and transmits an enrollment verification request containing healthcare setting information to the drug distribution management database manager. The drug distribution management database manager compares the information contained within the enrollment verification request to healthcare setting information in the healthcare setting sub-database 1012. If the healthcare setting 1003 is enrolled in the healthcare setting sub-database, the drug distribution management database manager 1006 can transmit a positive enrollment verification to the distributor 1005, which authorizes the distributor to provide the oral immunotherapy treatment kit to the healthcare setting 1003, as indicated at 1022. In some embodiments, verification occurs automatically; for example, the verification request may be received by a computer system communicatively connected to the drug distribution management database, which is automatically accessed to compare the information in the verification request to the information in the database. If a match between the information in the verification request and the information in the database is determined, an enrollment status may be automatically generated. In some embodiments, the enrollment status is automatically transmitted to the healthcare setting in response to receiving the request. The healthcare setting 1003 (such as by the prescriber 1001) then use doses of the drug in the oral immunotherapy treatment kit to administer the first dose of the drug at the selected level to the patient 1002, as shown at 1023. The patient 1002 is monitored at the healthcare setting 1003 (such as by the prescriber 1001) before the prescriber 1001 provides a prescription or request 1024 for a pharmacy 1004 to provide a plurality of doses of the drug at the same dose level for the patient 1002. The pharmacy 1004 receives the prescription or request 1024, and may provide the plurality of doses of the drug to the healthcare setting 1003 (at 1025) or the patient 1002 (at 1026) only after verifying enrollment of the patient 1002 in the patient sub-database 1011 and: i) the healthcare setting 1003 in the healthcare setting sub-database 1012 and the prescriber 1001 in the prescribe sub-database 1010; or ii) the prescriber 1001 in the prescriber sub-database 1010, as indicated at 1027. The pharmacy can submit a healthcare setting enrollment request containing healthcare setting information, a prescriber enrollment request containing prescriber information, and/or a patient enrollment verification request containing patient information to the drug distribution management database manager 1006. The drug distribution management database manager 1006 can compare the received information to the information in the drug distribution management database 1008, and can provide an enrollment verification to the pharmacy 1004, which authorizes the pharmacy 1004 to provide the drug to the patient 1002 or healthcare setting 1003. In some embodiments, verification occurs automatically; for example, the verification request may be received by a computer system communicatively connected to the drug distribution management database, which is automatically accessed to compare the information in the verification request to the information in the database. If a match between the information in the verification request and the information in the database is determined, an enrollment status may be automatically generated. In some embodiments, the enrollment status is automatically transmitted to the pharmacy in response to receiving the request. The pharmacy 1004 should only provide a single dose level of the drug for any given patient 1002 at a time.

Additional embodiments of the drug distribution and treatment methods are described below. Although the following discussion includes headings, it is understood that the headings are provided for organizational purposes only and various aspects of the described methods can be combined with aspects of other described methods.

### Initial Dose Escalation Kit

The initial dose escalation kit is provided by the pharmacy to the healthcare setting so that the healthcare setting can initiate treatment (such as by the prescriber/healthcare provider) of the patient for the food allergy. Each of these entities (the pharmacy, the healthcare provider, the healthcare setting, and the patient) must be enrolled in the in the drug distribution management database before the initial dose escalation kit is provided by the pharmacy to the healthcare setting. Further, the initial dose escalation kit is provided for a specific patient associated with the initial dose escalation kit, and the healthcare setting (and/or the healthcare provider) should not use the initial dose escalation kit associated with one patient for initiating treatment of another patient.

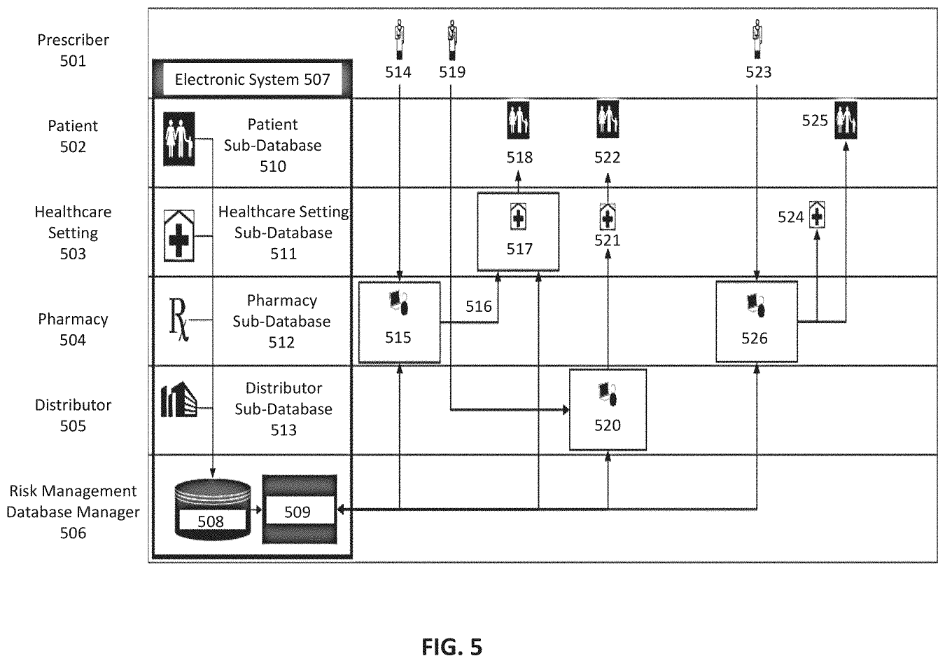

An exemplary process, which may be a computer-implemented process, for providing the initial dose escalation kit containing the drug is shown in FIG. 6. At step 602, the pharmacy, which must be enrolled in the drug distribution management database, receives a request to provide to the healthcare setting an initial dose escalation kit associated with an identified patient. A healthcare provider (which may be associated with the healthcare setting) can generate a prescription for the initial dose escalation kit. The prescription may be provided to the patient to provide to the pharmacy, or may be provided directly to the pharmacy. The prescription should include patient information for the patient to be treated, healthcare setting information for the healthcare setting that will treat the patient, and an indication that the initial dose escalation kit should be provided.

Once the pharmacy receives the prescription or request for the initial dose escalation kit at step 602, the pharmacy must be authorized to distribute the initial dose escalation kit prior to providing the initial dose escalation kit to the healthcare setting. To obtain the authorization, the pharmacy must verify that the patient identified in the request and the healthcare setting identified in the request are each enrolled in the drug distribution management database, at step 604. In a further embodiment, the pharmacy must verify that the patient's prescriber (such as a healthcare provider) is also enrolled in the drug distribution management database at step 604. The pharmacy can transmit an enrollment verification request containing patient information (i.e., a patient enrollment verification request) and an enrollment verification request containing healthcare setting information (i.e., a healthcare setting enrollment verification request) to a drug distribution management database manager. The pharmacy may also transmit an enrollment verification request containing prescriber information (i.e., a prescriber enrollment verification request, also termed a healthcare provider enrollment verification request). The enrollment verification requests may be transmitted, for example, by phone, fax, email, or another suitable electronic device.

By way of example, a computer implemented method of verifying enrollment of the patient and the healthcare setting in the drug distribution management database can include transmitting, at an electronic device associated with the pharmacy enrolled in the drug distribution management database for the drug, to a second electronic device communicatively coupled to the drug distribution management database, the healthcare setting enrollment verification request and the patient enrollment verification request. The pharmacy must be enrolled in the drug distribution management system, and may have unique credentials that allows the pharmacy to make the request. The second electronic device communicatively coupled to the drug distribution management database can receive the enrollment verification requests from the first electronic device associated with the pharmacy, and can generate an enrollment status for each of the healthcare setting and the patient. For example, the second electronic device can access the drug distribution management database, compare the healthcare setting information in the healthcare setting enrollment verification request to the healthcare setting information in the drug distribution management database, and obtain a positive healthcare setting enrollment status by identifying a match between the healthcare setting information in the healthcare setting enrollment verification request and the healthcare setting information in the drug distribution management database (or, alternatively, obtaining a negative healthcare setting enrollment status by failing to identify a match between the healthcare setting information in the healthcare setting enrollment verification request and the healthcare setting information in the drug distribution management database). Similarly, the second electronic device can access the drug distribution management database, compare the patient information in the patient enrollment verification request to the patient information in the drug distribution management database, and obtain a positive patient enrollment status by identifying a match between the patient information in the patient enrollment verification request and the patient information in the drug distribution management database (or, alternatively, obtaining a negative patient enrollment status by failing to identify a match between the patient information in the patient enrollment verification request and the patient information in the drug distribution management database). The second electronic device can then send, to the electronic device associated with the pharmacy, the healthcare setting enrollment status and the patient enrollment status, wherein receipt of a positive healthcare setting enrollment status and a positive patient enrollment status authorizes the pharmacy to distribute the initial dose escalation kit. In some embodiments, verification occurs automatically; for example, the verification request may be received by a computer system communicatively connected to the drug distribution management database, which is automatically accessed to compare the information in the verification request to the information in the database. If a match between the information in the verification request and the information in the database is determined, an enrollment status may be automatically generated. In some embodiments, the enrollment status is automatically transmitted to the pharmacy in response to receiving the request.

By way of example, a computer implemented method of verifying enrollment of the patient, the healthcare provider, and the healthcare setting in the drug distribution management database can include transmitting, at an electronic device associated with the pharmacy enrolled in the drug distribution management database for the drug, to a second electronic device communicatively coupled to the drug distribution management database, the healthcare setting enrollment verification request, the healthcare provider enrollment verification request, and the patient enrollment verification request. The pharmacy must be enrolled in the drug distribution management system, and may have unique credentials that allows the pharmacy to make the request. The second electronic device communicatively coupled to the drug distribution management database can receive the enrollment verification requests from the first electronic device associated with the pharmacy, and can generate an enrollment status for each of the healthcare setting, the healthcare provider, and the patient. For example, the second electronic device can access the drug distribution management database, compare the healthcare setting information in the healthcare setting enrollment verification request to the healthcare setting information in the drug distribution management database, and obtain a positive healthcare setting enrollment status by identifying a match between the healthcare setting information in the healthcare setting enrollment verification request and the healthcare setting information in the drug distribution management database (or, alternatively, obtaining a negative healthcare setting enrollment status by failing to identify a match between the healthcare setting information in the healthcare setting enrollment verification request and the healthcare setting information in the drug distribution management database). Similarly, the second electronic device can access the drug distribution management database, compare the patient information in the patient enrollment verification request to the patient information in the drug distribution management database, and obtain a positive patient enrollment status by identifying a match between the patient information in the patient enrollment verification request and the patient information in the drug distribution management database (or, alternatively, obtaining a negative patient enrollment status by failing to identify a match between the patient information in the patient enrollment verification request and the patient information in the drug distribution management database). Further, the second electronic device can access the drug distribution management database, compare the healthcare provider information in the healthcare provider enrollment verification request to the healthcare provider information in the drug distribution management database, and obtain a positive healthcare provider enrollment status by identifying a match between the healthcare provider information in the healthcare provider enrollment verification request and the healthcare provider information in the drug distribution management database (or, alternatively, obtaining a negative healthcare provider enrollment status by failing to identify a match between the healthcare provider information in the healthcare provider enrollment verification request and the healthcare provider information in the drug distribution management database). The second electronic device can then send, to the electronic device associated with the pharmacy, the healthcare setting enrollment status, the healthcare provider status, and the patient enrollment status, wherein receipt of a positive healthcare setting enrollment status, a positive healthcare provider enrollment status, and a positive patient enrollment status authorizes the pharmacy to distribute the initial dose escalation kit. In some embodiments, verification occurs automatically; for example, the verification request may be received by a computer system communicatively connected to the drug distribution management database, which is automatically accessed to compare the information in the verification request to the information in the database. If a match between the information in the verification request and the information in the database is determined, an enrollment status may be automatically generated. In some embodiments, the enrollment status is automatically transmitted to the pharmacy in response to receiving the request.

Referring to step 606 of FIG. 6, once the pharmacy verifies enrollment of the healthcare setting and the patient in the drug distribution management database (for example, by receiving a positive enrollment status for each of the healthcare setting and the patient), the pharmacy can provide an initial dose escalation kit to the healthcare setting, wherein the initial dose escalation kit is associated with the patient for which enrollment in the drug distribution management database was verified. In a further embodiment, once the pharmacy verifies enrollment of the healthcare setting, the healthcare provider, and the patient in the drug distribution management database (for example, by receiving a positive enrollment status for each of the healthcare setting, healthcare provider, and the patient), the pharmacy can provide, at step 606, an initial dose escalation kit to the healthcare setting, wherein the initial dose escalation kit is associated with the patient for which enrollment in the drug distribution management database was verified. The initial dose escalation kit is specific for the patient, and should not be used to administer the drug to any other patient. In some embodiments, the pharmacy generates a label with a patient identifier (which may be some or all of the information used to verify patient enrollment in the drug distribution management database), and affixes the label to the initial dose escalation kit. The label is a clear indication that the initial dose escalation kit is associated with the patient, and that the labeled initial dose escalation kit should not be used to treat any other patient. The healthcare setting can further verify that the correct initial dose escalation kit is being used with the correct patient by comparing the patient identifier on the initial dose escalation kit with patient information for that patient.

Once the healthcare setting receives the initial dose escalation kit, the healthcare setting can initiate treatment (such as by the prescriber/healthcare provider) of the patient for the food allergy by administering the doses of the drug in the initial dose escalation kit in accordance with the oral immunotherapy dosing schedule (that is, in accordance with the initial dose escalation phase schedule). The initial dose escalation kit includes a plurality of escalating doses, and the patient should be monitored (such as by the prescriber/healthcare provider) for an allergic event during a monitoring period (e.g., an hour or more) after the administration of each dose in the initial dose escalation kit.

In some embodiments, a method of providing a drug used to treat a food allergy by oral immunotherapy to a patient includes (a) receiving, at a pharmacy enrolled in a drug distribution management database for the drug, a request to provide to a healthcare setting an initial dose escalation kit associated with an identified patient, the initial dose escalation kit comprising a plurality of different doses of the drug according to an initial dose escalation schedule; (b) verifying enrollment of the identified patient in a drug distribution management database for the drug; (c) verifying enrollment of the healthcare setting in the drug distribution management database; and (d) providing, to the healthcare setting, the initial dose escalation kit associated with the identified patient.

In some embodiments, a method of providing a drug used to treat a food allergy by oral immunotherapy to a patient includes (a) receiving, at a pharmacy enrolled in a drug distribution management database for the drug, a request to provide to a healthcare setting an initial dose escalation kit associated with an identified patient, the initial dose escalation kit comprising a plurality of different doses of the drug according to an initial dose escalation schedule; (b) verifying enrollment of the identified patient in a drug distribution management database for the drug; (c) verifying enrollment of the healthcare setting in the drug distribution management database; (d) verifying enrollment of the healthcare provider in the drug distribution management database; and (e) providing, to the healthcare setting, the initial dose escalation kit associated with the identified patient.

### Oral Immunotherapy Treatment Kit

As discussed herein, the oral immunotherapy treatment kit includes a plurality of doses of the drug at each of the different dose levels used in accordance with the oral immunotherapy schedule during the up-dosing phase. The first dose of each new dosing level administered to the patient originates from the oral immunotherapy treatment kit.

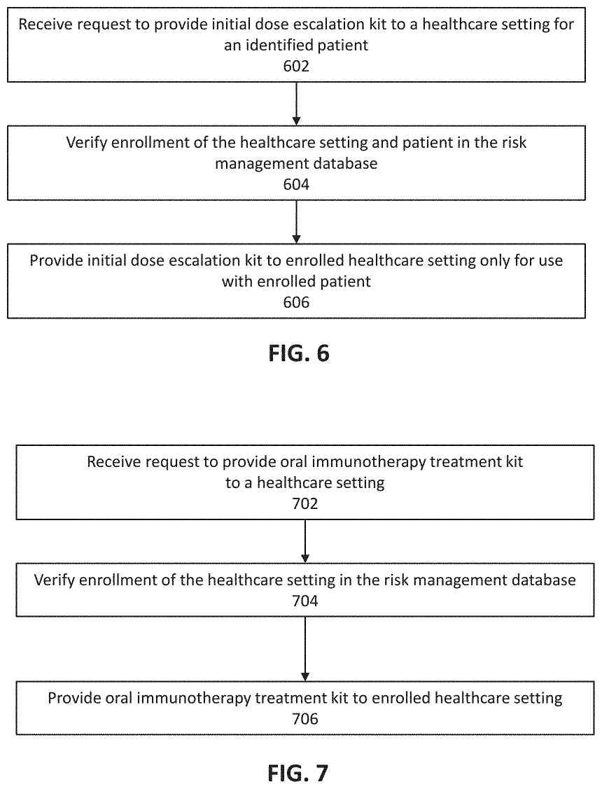

The healthcare setting receives the oral immunotherapy treatment kit from a distributor enrolled in the drug distribution management database only if the healthcare setting is enrolled in the drug distribution management database. FIG. 7 illustrates an exemplary method for providing the oral immunotherapy treatment kit to a healthcare setting. At step 702, the distributor receives a request from a healthcare setting to provide the oral immunotherapy treatment kit. At step 704, the distributor verifies enrollment of the healthcare setting in the drug distribution management database for the drug. The distributor can transmit an enrollment verification request containing healthcare setting information (i.e., a healthcare setting enrollment verification request) to a drug distribution management database manager. The enrollment verification requests may be transmitted, for example, by phone, fax, email, or other suitable electronic device.

By way of example, a computer-implement method of verifying enrollment of the healthcare setting in the drug distribution management database can include transmitting, at an electronic device associated with the distributor enrolled in the drug distribution management database for the drug, to a second electronic device communicatively coupled to the drug distribution management database, the healthcare setting enrollment verification request. The distributor must be enrolled in the drug distribution management system, and may have unique credentials that allows the distributor to make the request. The second electronic device communicatively coupled to the drug distribution management database can receive the enrollment verification requests from the first electronic device associated with the distributor, and can generate an enrollment status for the healthcare setting. For example, the second electronic device can access the drug distribution management database, compare the healthcare setting information in the healthcare setting enrollment verification request to the healthcare setting information in the drug distribution management database, and obtain a positive healthcare setting enrollment status by identifying a match between the healthcare setting information in the healthcare setting enrollment verification request and the healthcare setting information in the drug distribution management database (or, alternatively, obtaining a negative healthcare setting enrollment status by failing to identify a match between the healthcare setting information in the healthcare setting enrollment verification request and the healthcare setting information in the drug distribution management database). The second electronic device can then send, to the electronic device associated with the distributor, the healthcare setting enrollment status, wherein receipt of a positive healthcare setting enrollment status authorizes the distributor to provide the oral immunotherapy treatment kit to the healthcare setting. In some embodiments, verification occurs automatically; for example, the verification request may be received by a computer system communicatively connected to the drug distribution management database, which is automatically accessed to compare the information in the verification request to the information in the database. If a match between the information in the verification request and the information in the database is determined, an enrollment status may be automatically generated. In some embodiments, the enrollment status is automatically transmitted to the distributor in response to receiving the request.

Referring to step 706 of FIG. 7, once the distributor verifies enrollment of the healthcare setting in the drug distribution management database (for example, by receiving a positive enrollment status for each of the healthcare setting), the distributor can provide the oral immunotherapy treatment kit to the healthcare setting.

Once the healthcare setting receives the oral immunotherapy treatment kit, the healthcare setting can administer the first dose of each up-dosing level to the patient to start treatment at that dosing level, in accordance with an oral immunotherapy dosing schedule. The patient should be monitored for an allergic event during a monitoring period (e.g., an hour or more) after the administration of the dose.

In some embodiments, a method of providing an oral immunotherapy treatment kit comprising a plurality of doses of a drug for treating a food allergy at different up-dosing levels, includes (a) receiving, at a distributor enrolled in a drug distribution management database for the drug, a request from a healthcare setting for the oral immunotherapy treatment kit; (b) verifying enrollment of the healthcare setting in the drug distribution management database for the drug; and (c) providing the oral immunotherapy treatment kit to the healthcare setting only if the healthcare setting is enrolled in the drug distribution management database.

### Up-Dosing and Maintenance Doses

The patient being treated for the food allergy by oral immunotherapy receives a plurality of doses of the drug at each dose level (i.e., during the up-dosing phase or the maintenance level during the maintenance phase) for administration outside of the healthcare setting. The patient may only receive doses for a single dose level at a time, and the first dose of each dose level is administered to the patient in the healthcare setting (such as by the prescriber/healthcare provider).

The plurality of doses at each level are provided by a pharmacy to the patient (or parent or guardian of the patient) or to the healthcare setting, which may provide the doses to the patient (or parent or guardian of the patient). The healthcare provider (i.e., the prescriber) or healthcare setting can generate a prescription or request for the drug at the selected dose level for the patient. In some embodiments, the prescription for that dose level is provided only if the patient does not experience a clinically significant allergic response to the drug. In some embodiments, the prescription for that dose level is provided only if the patient does not experience a moderate or severe allergic response to the drug. In some embodiments, the prescription for that dose level is provided only if the patient does not experience a severe allergic response to the drug. In some embodiments, the prescription for that dose level is provided only if the patient does not experience a systemic allergic response to the drug.

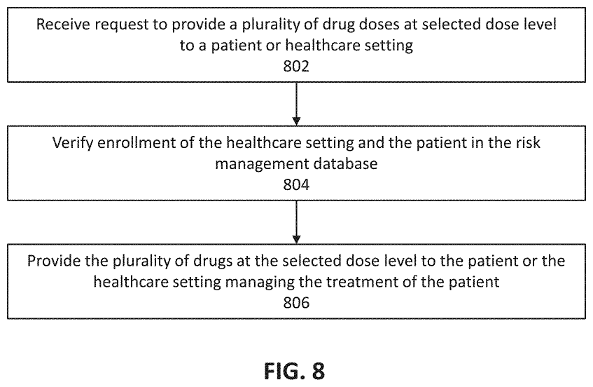

FIG. 8 shows an exemplary process for distributing the plurality of doses of the drug at the selected dose level is by a pharmacy, which is enrolled in the drug distribution management database. At step 802, the pharmacy, which must be enrolled in the drug distribution management database, receives a prescription or request to provide to the healthcare setting or patient a plurality of doses of the drug at the dose level indicated on the prescription or request. The prescription or request may further indicate how many doses of the drug should be provided, or an administration frequency (e.g., daily) and duration (e.g., two weeks) for administering the drug. A healthcare provider (which may be associated with the healthcare setting) can generate the request or prescription. The prescription or request may be provided to the patient to provide to the pharmacy, or may be provided directly to the pharmacy. In some embodiments, the prescription or request should include patient information for the patient to be treated and healthcare setting information for the healthcare setting. In some embodiments, the prescription or request should include patient information for the patient to be treated and healthcare provider information for the healthcare provider.

Once the pharmacy receives the prescription or request, the pharmacy must be authorized to provide the plurality of doses at the selected drug level to the patient or the healthcare setting. In some embodiments, to obtain the authorization, the pharmacy must verify that the patient identified in the request and the healthcare setting identified in the request are each enrolled in the drug distribution management database, at step 804. The pharmacy can transmit an enrollment verification request containing patient information (i.e., a patient enrollment verification request) and an enrollment verification request containing healthcare setting information (i.e., a healthcare setting enrollment verification request) to a drug distribution management database manager. The enrollment verification requests may be transmitted, for example, by phone, fax, email, or another suitable electronic device. In some embodiments, to obtain the authorization, the pharmacy must verify that the patient identified in the request and the healthcare provider identified in the request are each enrolled in the drug distribution management database. The pharmacy can transmit an enrollment verification request containing patient information (i.e., a patient enrollment verification request) and an enrollment verification request containing healthcare provider information (i.e., a healthcare provider enrollment verification request) to a drug distribution management database manager. The enrollment verification requests may be transmitted, for example, by phone, fax, email, or another suitable electronic device.

By way of example, a computer implemented method of verifying enrollment of the patient and the healthcare setting in the drug distribution management database can include transmitting, at an electronic device associated with the pharmacy enrolled in the drug distribution management database for the drug, to a second electronic device communicatively coupled to the drug distribution management database, the healthcare setting enrollment verification request and the patient enrollment verification request. The pharmacy must be enrolled in the drug distribution management system, and may have unique credentials that allows the pharmacy to make the request. The second electronic device communicatively coupled to the drug distribution management database can receive the enrollment verification requests from the first electronic device associated with the pharmacy, and can generate an enrollment status for each of the healthcare setting and the patient. For example, the second electronic device can access the drug distribution management database, compare the healthcare setting information in the healthcare setting enrollment verification request to the healthcare setting information in the drug distribution management database, and obtain a positive healthcare setting enrollment status by identifying a match between the healthcare setting information in the healthcare setting enrollment verification request and the healthcare setting information in the drug distribution management database (or, alternatively, obtaining a negative healthcare setting enrollment status by failing to identify a match between the healthcare setting information in the healthcare setting enrollment verification request and the healthcare setting information in the drug distribution management database). Similarly, the second electronic device can access the drug distribution management database, compare the patient information in the patient enrollment verification request to the patient information in the drug distribution management database, and obtain a positive patient enrollment status by identifying a match between the patient information in the patient enrollment verification request and the patient information in the drug distribution management database (or, alternatively, obtaining a negative patient enrollment status by failing to identify a match between the patient information in the patient enrollment verification request and the patient information in the drug distribution management database). The second electronic device can then send, to the electronic device associated with the pharmacy, the healthcare setting enrollment status and the patient enrollment status, wherein receipt of a positive healthcare setting enrollment status and a positive patient enrollment status authorizes the pharmacy to distribute the initial dose escalation kit. In some embodiments, verification occurs automatically; for example, the verification request may be received by a computer system communicatively connected to the drug distribution management database, which is automatically accessed to compare the information in the verification request to the information in the database. If a match between the information in the verification request and the information in the database is determined, an enrollment status may be automatically generated. In some embodiments, the enrollment status is automatically transmitted to the pharmacy in response to receiving the request.

By way of example, a computer implemented method of verifying enrollment of the patient and the healthcare provider in the drug distribution management database can include transmitting, at an electronic device associated with the pharmacy enrolled in the drug distribution management database for the drug, to a second electronic device communicatively coupled to the drug distribution management database, the healthcare provider enrollment verification request and the patient enrollment verification request. The pharmacy must be enrolled in the drug distribution management system, and may have unique credentials that allows the pharmacy to make the request. The second electronic device communicatively coupled to the drug distribution management database can receive the enrollment verification requests from the first electronic device associated with the pharmacy, and can generate an enrollment status for each of the healthcare setting and the patient. For example, the second electronic device can access the drug distribution management database, compare the healthcare provider information in the healthcare provider enrollment verification request to the healthcare provider information in the drug distribution management database, and obtain a positive healthcare provider enrollment status by identifying a match between the healthcare provider information in the healthcare provider enrollment verification request and the healthcare provider information in the drug distribution management database (or, alternatively, obtaining a negative healthcare provider enrollment status by failing to identify a match between the healthcare provider information in the healthcare provider enrollment verification request and the healthcare provider information in the drug distribution management database). Similarly, the second electronic device can access the drug distribution management database, compare the patient information in the patient enrollment verification request to the patient information in the drug distribution management database, and obtain a positive patient enrollment status by identifying a match between the patient information in the patient enrollment verification request and the patient information in the drug distribution management database (or, alternatively, obtaining a negative patient enrollment status by failing to identify a match between the patient information in the patient enrollment verification request and the patient information in the drug distribution management database). The second electronic device can then send, to the electronic device associated with the pharmacy, the healthcare provider enrollment status and the patient enrollment status, wherein receipt of a positive healthcare provider enrollment status and a positive patient enrollment status authorizes the pharmacy to distribute the initial dose escalation kit. In some embodiments, verification occurs automatically; for example, the verification request may be received by a computer system communicatively connected to the drug distribution management database, which is automatically accessed to compare the information in the verification request to the information in the database. If a match between the information in the verification request and the information in the database is determined, an enrollment status may be automatically generated. In some embodiments, the enrollment status is automatically transmitted to the pharmacy in response to receiving the request.

Referring to step 806 of FIG. 8, once the pharmacy verifies enrollment of the healthcare setting and the patient in the drug distribution management database (for example, by receiving a positive enrollment status for each of the healthcare setting and the patient), the pharmacy can provide the plurality of doses at the indicated dose level to the patient or the healthcare setting. In a further embodiment, once the pharmacy verifies enrollment of the healthcare provider and the patient in the drug distribution management database (for example, by receiving a positive enrollment status for each of the healthcare provider and the patient), the pharmacy can provide the plurality of doses at the indicated dose level to the patient or the healthcare setting. However, the pharmacy is not authorized to provide to the patient or the healthcare setting for use by the patient a plurality of doses at different dose levels. The provided plurality of doses is specific for the patient, and should not be used to administer the drug to any other patient. In some embodiments, the pharmacy generates a label with a patient identifier (which may be some or all of the information used to verify patient enrollment in the drug distribution management database), and affixes the label to a container containing the plurality of doses. The label is a clear indication that the doses are associate with the patient.

This process of the pharmacy receiving authorization to distribute the plurality of doses at the selected dose level is preferably repeated each time the plurality of doses at the selected dose level is provided to the patient (or the healthcare setting to be used to treat the patient). In some embodiments, if the prescriber or healthcare setting recommends or prescribes a different dose level (or a repeat of the same dose level), the pharmacy must again verify enrollment of the patient and the healthcare setting in the drug distribution management database before authorization to provide the drug is given. In some embodiments, if the prescriber or healthcare setting recommends or prescribes a different dose level (or a repeat of the same dose level), the pharmacy must again verify enrollment of the patient and the healthcare provider in the drug distribution management database before authorization to provide the drug is given.

In some embodiments, a method of providing a drug used to treat a food allergy by oral immunotherapy to a patient includes (a) receiving, at a pharmacy enrolled in a drug distribution management database for the drug, a prescription authorized by the healthcare setting to provide a plurality of doses of the drug at an up-dosing level according to an up-dosing schedule or a plurality of doses of the drug at a maintenance level; (b) verifying enrollment of the identified patient in a drug distribution management database for the drug; (c) verifying enrollment of the healthcare setting in the drug distribution management database; and (d) providing, to the patient or the healthcare setting, the plurality of doses of the drug at the up-dosing level or the plurality of doses of the drug at the maintenance level.

### Electronic Devices and Systems

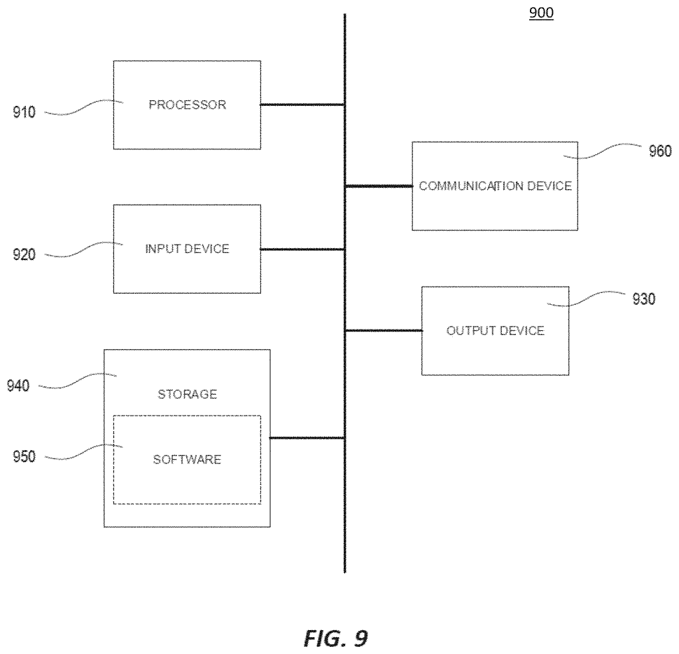

The methods described herein may be implemented on computer systems or other electronic devices. FIG. 9 illustrates an example of a computing device in accordance with one embodiment. Device 900 can be a host computer connected to a network. Device 900 can be a client computer or a server. As shown in FIG. 9, device 900 can be any suitable type of microprocessor-based device, such as a personal computer, workstation, server or handheld computing device (portable electronic device) such as a phone or tablet. The device can include, for example, one or more of processor 910, input device 920, output device 930, storage or memory 940, and communication device 960. Input device 920 and output device 930 can generally correspond to those described above, and can either be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage, such as an electrical, magnetic or optical memory including a RAM, cache, hard drive, or removable storage disk. Communication device 960 can include any suitable device capable of transmitting and receiving signals over a network, such as a network interface chip or device. The components of the computer can be connected in any suitable manner, such as via a physical bus or wirelessly.

Software 950, which can be stored in storage 940 and executed by processor 910, can include, for example, the programming that embodies the functionality of the present disclosure (e.g., as embodied in the devices as described above).

Software 950 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 940, that can contain or store programming for use by or in connection with an instruction execution system, apparatus, or device.

Software 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 900 may be connected to a network, which can be any suitable type of interconnected communication system. The network can implement any suitable communications protocol and can be secured by any suitable security protocol. For example, any of the databases described herein can be password protected, optionally using a two-factor identification protocol. The password may be required, for example, to access, submit, upload, or alter information in a database The network can comprise network links of any suitable arrangement that can implement the transmission and reception of network signals, such as wireless network connections, T1 or T3 lines, cable networks, DSL, or telephone lines.

Device 900 can implement any operating system suitable for operating on the network. Software 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, or a mobile application (such as a mobile phone or tablet application), for example.

Steps for any of the computer-implemented methods described herein, or any of the methods provided by program instructions described herein, may be automatic, which can help facilitate efficient drug distribution management by speeding up the process and minimizing the possibility of human error when performing steps. The speed of verifying enrollment is substantially beneficial for the drug distribution method because the dose level of the drug administered to patients according to the oral immunotherapy regimen changes several times during treatment, and the patients need to quickly have access the “next” dose level to maintain the regular dosing (e.g., daily dosing). Further, because the drug distribution system helps ensure each patient is being provided with the correct dose level of the drug, and that no patient is receiving the drug that should not receive the drug, it is important to minimize human error. By way of example, in some embodiments, one or more verification steps may occur automatically. For example, the verification request may be received by a computer system communicatively connected to the drug distribution management database, which is automatically accessed to compare the information in the verification request to the information in the database. If a match between the information in the verification request and the information in the database is determined, an enrollment status may be automatically generated. In some embodiments, the enrollment status is automatically transmitted to the entity transmitting the verification request in response to the computer system receiving the verification request.

In some embodiments, a computer-implemented method for authorizing the provision of a drug to a healthcare setting for treatment of a food allergy by oral immunotherapy includes (a) receiving, at a first electronic device communicatively coupled to a drug distribution management database for the drug and from a second electronic device associated with a pharmacy enrolled in the drug distribution management database, a healthcare setting enrollment verification request comprising healthcare setting information for a healthcare setting and a patient enrollment verification request comprising patient information for a patient; (b) generating, at the first electronic device, a healthcare setting enrollment status, comprising (i) accessing the drug distribution management database, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generating, at the first electronic device, a patient enrollment status, comprising (i) accessing the drug distribution management database, (ii) comparing the patient information in the patient enrollment verification request to enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule.

In some embodiments, a computer-implemented method for authorizing the provision of a drug to a healthcare setting for treatment of a food allergy by oral immunotherapy includes (a) receiving, at a first electronic device communicatively coupled to a drug distribution management database for the drug and from a second electronic device associated with a pharmacy enrolled in the drug distribution management database, a healthcare setting enrollment verification request comprising healthcare setting information for a healthcare setting, a healthcare provider enrollment verification request comprising healthcare provider information for a healthcare provider, and a patient enrollment verification request comprising patient information for a patient; (b) generating, at the first electronic device, a healthcare setting enrollment status, comprising (i) accessing the drug distribution management database, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generating, at the first electronic device, a patient enrollment status, comprising (i) accessing the drug distribution management database, (ii) comparing the patient information in the patient enrollment verification request to enrolled patient information in the drug distribution management database, and (iii) obtaining a positive patient enrollment status by identifying a match between the patient information in the healthcare setting enrollment verification request and the patient information in the drug distribution management database, or obtaining a negative patient enrollment status if no match between the patient information in the patient enrollment verification request and the patient information in the drug distribution management database is identified; (d) generating, at the first electronic device, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; and (e) sending, to the second electronic device, the healthcare setting enrollment status, healthcare provider enrollment status, and the patient enrollment status, wherein receipt of a positive healthcare setting enrollment status, a positive healthcare provider enrollment status, and a positive patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule.

In some embodiments, there is a computer-implemented method for authorizing the provision of a drug to a healthcare setting for the treatment of a food allergy by oral immunotherapy includes (a) receiving, at a first electronic device communicatively coupled to a drug distribution management database for the drug and from a second electronic device associated with a pharmacy enrolled in the drug distribution management database, a healthcare setting enrollment verification request comprising healthcare setting information for a healthcare setting and a patient enrollment verification request comprising patient information for a patient; (b) generating, at the first electronic device, a healthcare setting enrollment status, comprising (i) accessing the drug distribution management database, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generating, at the first electronic device, a patient enrollment status, comprising (i) accessing the drug distribution management database, (ii) comparing the patient information in the patient enrollment verification request to enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting or the patient a plurality of doses of the drug at a single treatment dose level according to an oral immunotherapy dosing schedule.

In some embodiments, there is a computer-implemented method for authorizing the provision of a drug to a healthcare setting for the treatment of a food allergy by oral immunotherapy includes (a) receiving, at a first electronic device communicatively coupled to a drug distribution management database for the drug and from a second electronic device associated with a pharmacy enrolled in the drug distribution management database, a healthcare provider enrollment verification request comprising healthcare provider information for a healthcare provider and a patient enrollment verification request comprising patient information for a patient; (b) generating, at the first electronic device, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; (c) generating, at the first electronic device, a patient enrollment status, comprising (i) accessing the drug distribution management database, (ii) comparing the patient information in the patient enrollment verification request to enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare provider enrollment status and the patient enrollment status, wherein receipt of a positive healthcare provider enrollment status and a positive patient enrollment status by the pharmacy authorizes the pharmacy to provide to the healthcare setting, the healthcare provider, or the patient a plurality of doses of the drug at a single treatment dose level according to an oral immunotherapy dosing schedule.

In some embodiments, a computer-implemented method for authorizing the provision of a drug to a healthcare setting for the treatment of a food allergy by oral immunotherapy includes: (a) receiving, at a first electronic device communicatively coupled to a drug distribution management database for the drug and from a second electronic device associated with a distributor enrolled in the drug distribution management database, a pharmacy enrollment verification request comprising pharmacy information for a pharmacy; (b) generating, at the first electronic device, a pharmacy enrollment status, comprising (i) accessing the drug distribution management database, (ii) comparing the pharmacy information in the pharmacy enrollment verification request to enrolled pharmacy information in the drug distribution management database, and (iii) obtaining a positive pharmacy enrollment status by identifying a match between the pharmacy information in the pharmacy enrollment verification request and the pharmacy information in the drug distribution management database, or obtaining a negative pharmacy enrollment status if no match between the pharmacy information in the pharmacy enrollment verification request and the pharmacy information in the drug distribution management database is identified; and (c) sending, to the second electronic device, the pharmacy enrollment status, wherein receipt of a positive pharmacy enrollment status authorizes the distributor to provide the drug to the pharmacy.

In some embodiments, a computer-implemented method for authorizing the distribution of an oral immunotherapy treatment kit comprising a plurality of doses of a drug for treating a food allergy at different up-dosing levels to a healthcare setting includes (a) receiving, at a first electronic device communicatively coupled to a drug distribution management database for the drug and from a second electronic device associated with a distributor enrolled in the drug distribution management database, a healthcare setting enrollment verification request comprising healthcare setting information for a healthcare setting; (b) generating, at the first electronic device, a healthcare setting enrollment status, comprising (i) accessing the drug distribution management database, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; and (c) sending, to the second electronic device, the healthcare setting enrollment status, wherein receipt of a positive healthcare setting enrollment status authorizes the distributor to provide the oral immunotherapy treatment kit to the healthcare setting.

In some embodiments, a computer-implemented method for managing risk associated with a drug used for treating a food allergy by oral immunotherapy includes (a) authorizing the provision of an initial dose escalation kit comprising plurality of different doses of the drug according to an initial dose escalation schedule, comprising: (i) receiving, at a first electronic device communicatively coupled to a drug distribution management database for the drug and from a second electronic device associated with a pharmacy enrolled in the drug distribution management database, a first healthcare setting enrollment verification request comprising healthcare setting information for a healthcare setting and a first patient enrollment verification request comprising patient information for a patient; (ii) generating, at the first electronic device, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the first healthcare setting enrollment verification request and the healthcare setting information in the drug distribution management database, or obtaining a negative healthcare setting enrollment status if no match between the healthcare setting information in the first healthcare setting enrollment verification request and the healthcare setting information in the drug distribution management database is identified; (iii) generating, at the first electronic device, a first patient enrollment status, comprising (1) accessing the drug distribution management database, (2) comparing the patient information in the first patient enrollment verification request to enrolled patient information in the drug distribution management database, and (3) obtaining a positive patient enrollment status by identifying a match between the patient information in the first patient enrollment verification request and the patient information in the drug distribution management database, or obtaining a negative patient enrollment status if no match between the patient information in the first patient enrollment verification request and the patient information in the drug distribution management database is identified; and (iv) sending, to the second electronic device, the first healthcare setting enrollment status and the first patient enrollment status, wherein receipt of a positive first healthcare setting enrollment status and a positive first patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule; (b) authorizing the provision of a plurality of doses of the drug at a single treatment dose level according to an oral immunotherapy dosing schedule, comprising: (i) receiving, at the first electronic device from the second electronic device, a second healthcare setting enrollment verification request comprising healthcare setting information for a healthcare setting and a second patient enrollment verification request comprising patient information for a patient; (ii) generating, at the first electronic device, a second healthcare setting enrollment status, comprising (1) accessing the drug distribution management database, (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; (iii) generating, at the first electronic device, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare setting enrollment status and the second patient enrollment status, wherein receipt of a positive second healthcare setting enrollment status and a positive second patient enrollment status by the pharmacy authorizes the pharmacy to provide to the healthcare setting or the patient a plurality of doses of the drug at a single treatment dose level according to an oral immunotherapy dosing schedule; (c) repeating step (b) at a different treatment dose level, wherein the pharmacy is not authorized to simultaneously provide the drug at more than one treatment dose level for the patient; (d) authorizing the provision of an oral immunotherapy treatment kit comprising a plurality of doses of a drug for treating a food allergy at different up-dosing levels to a healthcare setting, comprising: (i) receiving, at the first electronic device and from a third electronic device associated with a distributor enrolled in the drug distribution management database, a third healthcare setting enrollment verification request comprising healthcare setting information for a healthcare setting; (ii) generating, at the first electronic device, a third healthcare setting enrollment status, comprising (1) accessing the drug distribution management database, and (2) comparing the healthcare setting information in the third healthcare setting enrollment verification request to enrolled healthcare setting information in the drug distribution management database, and (3) obtaining a positive healthcare setting enrollment status by identifying a match between the healthcare setting information in the third healthcare setting enrollment verification request and the healthcare setting information in the drug distribution management database, or obtaining a negative healthcare setting enrollment status if no match between the healthcare setting information in the third healthcare setting enrollment verification request and the healthcare setting information in the drug distribution management database is identified; and (iii) sending, to the third electronic device, the third healthcare setting enrollment status, wherein receipt of a positive third healthcare setting enrollment status authorizes the distributor to provide the oral immunotherapy treatment kit to the healthcare setting.

In some embodiments, a computer-implemented method for managing risk associated with a drug used for treating a food allergy by oral immunotherapy includes (a) authorizing the provision of an initial dose escalation kit comprising plurality of different doses of the drug according to an initial dose escalation schedule, comprising: (i) receiving, at a first electronic device communicatively coupled to a drug distribution management database for the drug and from a second electronic device associated with a pharmacy enrolled in the drug distribution management database, a first healthcare setting enrollment verification request comprising healthcare setting information for a healthcare setting, a first healthcare provider verification request comprising healthcare provider information for a healthcare provider, and a first patient enrollment verification request comprising patient information for a patient; (ii) generating, at the first electronic device, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the first healthcare setting enrollment verification request and the healthcare setting information in the drug distribution management database, or obtaining a negative healthcare setting enrollment status if no match between the healthcare setting information in the first healthcare setting enrollment verification request and the healthcare setting information in the drug distribution management database is identified; (iii) generating, at the first electronic device, a first patient enrollment status, comprising (1) accessing the drug distribution management database, (2) comparing the patient information in the first patient enrollment verification request to enrolled patient information in the drug distribution management database, and (3) obtaining a positive patient enrollment status by identifying a match between the patient information in the first patient enrollment verification request and the patient information in the drug distribution management database, or obtaining a negative patient enrollment status if no match between the patient information in the first patient enrollment verification request and the patient information in the drug distribution management database is identified; (iv) generating, at the first electronic device, a first healthcare provider enrollment status, comprising (1) accessing the drug distribution management database, (2) comparing the healthcare provider information in the first healthcare provider enrollment verification request to enrolled healthcare provider information in the first healthcare provider enrollment verification request to enrolled healthcare provider information in the drug distribution management database, and (3) obtaining a positive healthcare provider enrollment verification request by identifying a match between the healthcare provider information in the first healthcare provider enrollment verification request and the healthcare provider information in the drug distribution management database, or obtaining a negative healthcare provider enrollment status if no match between the healthcare provider information in the first healthcare provider enrollment verification request and the healthcare provider information in the drug distribution management database is identifying; and (v) sending, to the second electronic device, the first healthcare setting enrollment status, the first healthcare provider enrollment status, and the first patient enrollment status, wherein receipt of a positive first healthcare setting enrollment status, a positive first healthcare provider enrollment status, and a positive first patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule; (b) authorizing the provision of a plurality of doses of the drug at a single treatment dose level according to an oral immunotherapy dosing schedule, comprising: (i) receiving, at the first electronic device from the second electronic device, a second healthcare provider enrollment verification request comprising healthcare provider information for a healthcare provider and a second patient enrollment verification request comprising patient information for a patient; (ii) generating, at the first electronic device, a second healthcare provider enrollment status, comprising (1) accessing the drug distribution management database, (2) comparing the healthcare provider information in the second healthcare provider enrollment verification request to enrolled healthcare provider information in the drug distribution management database, and (3) obtaining a positive healthcare provider enrollment status by identifying a match between the healthcare provider information in the second healthcare provider enrollment verification request and the healthcare provider information in the drug distribution management database, or obtaining a negative healthcare provider enrollment status if no match between the healthcare provider information in the second healthcare provider enrollment verification request and the healthcare provider information in the drug distribution management database is identified; (iii) generating, at the first electronic device, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare provider enrollment status and the second patient enrollment status, wherein receipt of a positive second healthcare provider enrollment status and a positive second patient enrollment status by the pharmacy authorizes the pharmacy to provide to the healthcare setting or the patient a plurality of doses of the drug at a single treatment dose level according to an oral immunotherapy dosing schedule; (c) repeating step (b) at a different treatment dose level, wherein the pharmacy is not authorized to simultaneously provide the drug at more than one treatment dose level for the patient; (d) authorizing the provision of an oral immunotherapy treatment kit comprising a plurality of doses of a drug for treating a food allergy at different up-dosing levels to a healthcare setting, comprising: (i) receiving, at the first electronic device and from a third electronic device associated with a distributor enrolled in the drug distribution management database, a second healthcare setting enrollment verification request comprising healthcare setting information for a healthcare setting; (ii) generating, at the first electronic device, a second healthcare setting enrollment status, comprising (1) accessing the drug distribution management database, and (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; and (iii) sending, to the third electronic device, the second healthcare setting enrollment status, wherein receipt of a positive second healthcare setting enrollment status authorizes the distributor to provide the oral immunotherapy treatment kit to the healthcare setting.

In some embodiments, an electronic device includes one or more processors; a memory configured to store a drug distribution management database for a drug for treating a food allergy, the drug distribution management database comprising entries for enrolled patients comprising enrolled patient information, entries for enrolled healthcare settings comprising enrolled healthcare setting information, and entries for enrolled pharmacies comprising enrolled pharmacy information; and one or more programs stored on the memory configured to be executed by the one or more processors, the one or more programs including instructions for: (a) receiving, from a second electronic device associated with a pharmacy enrolled in the drug distribution management database, a healthcare setting enrollment verification request comprising healthcare setting information for a healthcare setting and a patient enrollment verification request comprising patient information for a patient; (b) generating, by the one or more processors, a healthcare setting enrollment status, comprising (i) accessing the drug distribution management database, (ii) comparing the healthcare setting information in the healthcare setting enrollment verification request to the healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generating, by the one or more processors, a patient enrollment status, comprising (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule.

In some embodiments, an electronic device includes one or more processors; a memory configured to store a drug distribution management database for a drug for treating a food allergy, the drug distribution management database comprising entries for enrolled patients comprising enrolled patient information, entries for enrolled healthcare settings comprising enrolled healthcare setting information, entries for enrolled healthcare providers comprising enrolled healthcare provider information, and entries for enrolled pharmacies comprising enrolled pharmacy information; and one or more programs stored on the memory configured to be executed by the one or more processors, the one or more programs including instructions for: (a) receiving, from a second electronic device associated with a pharmacy enrolled in the drug distribution management database, a healthcare setting enrollment verification request comprising healthcare setting information for a healthcare setting, a healthcare provider enrollment verification request comprising healthcare provider information for a healthcare provider, and a patient enrollment verification request comprising patient information for a patient; (b) generating, by the one or more processors, a healthcare setting enrollment status, comprising (i) accessing the drug distribution management database, (ii) comparing the healthcare setting information in the healthcare setting enrollment verification request to the healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generating, by the one or more processors, a healthcare provider enrollment status, comprising (i) accessing the drug distribution management database, (ii) comparing the healthcare provider information in the healthcare provider enrollment verification request to the healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; (d) generating, by the one or more processors, a patient enrollment status, comprising (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare setting enrollment status, the healthcare provider enrollment status, and the patient enrollment status, wherein receipt of a positive healthcare setting enrollment status, a positive healthcare provider enrollment status, and a positive patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule.

In some embodiments, an electronic device includes one or more processors; a memory configured to store a drug distribution management database for a drug for treating a food allergy, the drug distribution management database comprising entries for enrolled patients comprising enrolled patient information, entries for enrolled healthcare settings comprising enrolled healthcare setting information, and entries for enrolled pharmacies comprising enrolled pharmacy information; and one or more programs stored on the memory configured to be executed by the one or more processors, the one or more programs including instructions for: (a) receiving, from a second electronic device associated with a pharmacy enrolled in the drug distribution management database, a healthcare setting enrollment verification request comprising healthcare setting information for a healthcare setting and a patient enrollment verification request comprising patient information for a patient; (b) generating, by the one or more processors, a healthcare setting enrollment status, comprising (i) accessing the drug distribution management database, (ii) comparing the healthcare setting information in the healthcare setting enrollment verification request to the healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generating, by the one or more processors, a patient enrollment status, comprising (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting or the patient a plurality of doses of the drug at a single treatment dose level according to an oral immunotherapy dosing schedule.

In some embodiments, an electronic device includes one or more processors; a memory configured to store a drug distribution management database for a drug for treating a food allergy, the drug distribution management database comprising entries for enrolled patients comprising enrolled patient information, entries for enrolled healthcare providers comprising enrolled healthcare providers information, and entries for enrolled pharmacies comprising enrolled pharmacy information; and one or more programs stored on the memory configured to be executed by the one or more processors, the one or more programs including instructions for: (a) receiving, from a second electronic device associated with a pharmacy enrolled in the drug distribution management database, a healthcare provider enrollment verification request comprising healthcare provider information for a healthcare provider and a patient enrollment verification request comprising patient information for a patient; (b) generating, by the one or more processors, a healthcare provider enrollment status, comprising (i) accessing the drug distribution management database, (ii) comparing the healthcare provider information in the healthcare provider enrollment verification request to the healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; (c) generating, by the one or more processors, a patient enrollment status, comprising (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare provider enrollment status and the patient enrollment status, wherein receipt of a positive healthcare provider enrollment status and a positive patient enrollment status by the pharmacy authorizes the pharmacy to provide to the healthcare setting, the healthcare provider, or the patient a plurality of doses of the drug at a single treatment dose level according to an oral immunotherapy dosing schedule.

In some embodiments, an electronic device includes one or more processors; a memory configured to store a drug distribution management database for a drug for treating a food allergy, the drug distribution management database comprising entries for enrolled distributors comprising enrolled distributor information, and entries for enrolled pharmacies comprising enrolled pharmacy information; and one or more programs stored on the memory configured to be executed by the one or more processors, the one or more programs including instructions for: (a) receiving, by the one or more processors, a pharmacy enrollment verification request comprising pharmacy information for a pharmacy; (b) generating, by the one or more processors, a pharmacy enrollment status, comprising (i) accessing the drug distribution management database, (ii) comparing the pharmacy information in the pharmacy enrollment verification request to enrolled pharmacy information in the drug distribution management database, and (iii) obtaining a positive pharmacy enrollment status by identifying a match between the pharmacy information in the pharmacy enrollment verification request and the pharmacy information in the drug distribution management database, or obtaining a negative pharmacy enrollment status if no match between the pharmacy information in the pharmacy enrollment verification request and the pharmacy information in the drug distribution management database is identified; and (c) sending, to a second electronic device associated with the distributor, the pharmacy enrollment status, wherein receipt of a positive pharmacy enrollment status authorizes the distributor to provide the drug to the pharmacy.

In some embodiments, an electronic device includes one or more processors; a memory configured to store a drug distribution management database for a drug for treating a food allergy, the drug distribution management database comprising entries for enrolled distributors comprising enrolled distributor information, and entries for enrolled healthcare settings comprising enrolled healthcare setting information; and one or more programs stored on the memory configured to be executed by the one or more processors, the one or more programs including instructions for: (a) receiving, by the one or more processors, a healthcare setting enrollment verification request comprising healthcare setting information for a healthcare setting; (b) generating, by the one or more processors, a healthcare setting enrollment status, comprising (i) accessing the drug distribution management database, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; and (c) sending, to a second electronic device associated with the distributor, the healthcare setting enrollment status, wherein receipt of a positive healthcare setting enrollment status authorizes the distributor to provide the oral immunotherapy treatment kit to the healthcare setting.

In some embodiments, an electronic system includes one or more processors; a memory configured to store a drug distribution management database for a drug for treating a food allergy, the drug distribution management database comprising entries for enrolled patients comprising enrolled patient information, entries for enrolled healthcare settings comprising enrolled healthcare setting information, entries for enrolled pharmacies comprising enrolled pharmacy information, and entries for enrolled distributors comprising enrolled distributor information; and one or more programs stored on the memory configured to be executed by the one or more processors, the one or more programs including instructions for: (a) authorizing, by the one or more processors, the provision of an initial dose escalation kit comprising plurality of different doses of the drug according to an initial dose escalation schedule, comprising (i) receiving, by the one or more processors and from a second electronic device associated with a pharmacy enrolled in the drug distribution management database, a first healthcare setting enrollment verification request comprising healthcare setting information for a healthcare setting and a first patient enrollment verification request comprising patient information for a patient; (ii) generating, by the one or more processors, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the first healthcare setting enrollment verification request and the healthcare setting information in the drug distribution management database, or obtaining a negative healthcare setting enrollment status if no match between the healthcare setting information in the first healthcare setting enrollment verification request and the healthcare setting information in the drug distribution management database is identified; (iii) generating, by the one or more processors, a first patient enrollment status, comprising (1) accessing the drug distribution management database, and (2) comparing the patient information in the first patient enrollment verification request to enrolled patient information in the drug distribution management database, and (3) obtaining a positive patient enrollment status by identifying a match between the patient information in the first patient enrollment verification request and the patient information in the drug distribution management database, or obtaining a negative patient enrollment status if no match between the patient information in the first patient enrollment verification request and the patient information in the drug distribution management database is identified; and (iv) sending, to the second electronic device, the first healthcare setting enrollment status and the first patient enrollment status, wherein receipt of a positive first healthcare setting enrollment status and a positive first patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule; (b) authorizing, by the one or more processors, a plurality of doses of the drug at a single treatment dose level according to an oral immunotherapy dosing schedule, comprising: (i) receiving, by the one or more processors and from the second electronic device, a second healthcare setting enrollment verification request comprising healthcare setting information for a healthcare setting and a second patient enrollment verification request comprising patient information for a patient; (ii) generating, by the one or more processors, a second healthcare setting enrollment status, comprising (1) accessing the drug distribution management database, (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; (iii) generating, by the one or more processors, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare setting enrollment status and the second patient enrollment status, wherein receipt of a positive second healthcare setting enrollment status and a positive second patient enrollment status by the pharmacy authorizes the pharmacy to provide to the healthcare setting or the patient a plurality of doses of the drug at a single treatment dose level according to an oral immunotherapy dosing schedule; (c) repeating step (b) at a different treatment dose level, wherein the pharmacy is not authorized to simultaneously provide the drug at more than one treatment dose level for the patient; (d) authorizing, by the one or more processors, the provision of an oral immunotherapy treatment kit comprising a plurality of doses of a drug for treating a food allergy at different up-dosing levels to a healthcare setting, comprising: (i) receiving, by the one or more processors and from a third electronic device associated with a distributor enrolled in the drug distribution management database, a third healthcare setting enrollment verification request comprising healthcare setting information for a healthcare setting; (ii) generating, by the one or more processors, a third healthcare setting enrollment status, comprising (1) accessing the drug distribution management database, (2) comparing the healthcare setting information in the third healthcare setting enrollment verification request to enrolled healthcare setting information in the drug distribution management database, and (3) obtaining a positive healthcare setting enrollment status by identifying a match between the healthcare setting information in the third healthcare setting enrollment verification request and the healthcare setting information in the drug distribution management database, or obtaining a negative healthcare setting enrollment status if no match between the healthcare setting information in the third healthcare setting enrollment verification request and the healthcare setting information in the drug distribution management database is identified; and (iii) sending, to the third electronic device, the third healthcare setting enrollment status, wherein receipt of a positive third healthcare setting enrollment status authorizes the distributor to provide the oral immunotherapy treatment kit to the healthcare setting.

In some embodiments, an electronic system includes one or more processors; a memory configured to store a drug distribution management database for a drug for treating a food allergy, the drug distribution management database comprising entries for enrolled patients comprising enrolled patient information, entries for enrolled healthcare settings comprising enrolled healthcare setting information, entries for enrolled healthcare providers comprising enrolled healthcare provider information, entries for enrolled pharmacies comprising enrolled pharmacy information, and entries for enrolled distributors comprising enrolled distributor information; and one or more programs stored on the memory configured to be executed by the one or more processors, the one or more programs including instructions for: (a) authorizing, by the one or more processors, the provision of an initial dose escalation kit comprising plurality of different doses of the drug according to an initial dose escalation schedule, comprising (i) receiving, by the one or more processors and from a second electronic device associated with a pharmacy enrolled in the drug distribution management database, a first healthcare setting enrollment verification request comprising healthcare setting information for a healthcare setting, a first healthcare provider enrollment verification request comprising healthcare provider information for a healthcare provider, and a first patient enrollment verification request comprising patient information for a patient; (ii) generating, by the one or more processors, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the first healthcare setting enrollment verification request and the healthcare setting information in the drug distribution management database, or obtaining a negative healthcare setting enrollment status if no match between the healthcare setting information in the first healthcare setting enrollment verification request and the healthcare setting information in the drug distribution management database is identified; (iii) generating, by the one or more processors, a first healthcare provider enrollment status, comprising (1) accessing the drug distribution management database, (2) comparing the healthcare provider information in the first healthcare provider enrollment verification request to enrolled healthcare provider information in the drug distribution management database, and (3) obtaining a positive healthcare provider enrollment status by identifying a match between the healthcare provider information in the first healthcare provider enrollment verification request and the healthcare provider information in the drug distribution management database, or obtaining a negative healthcare provider enrollment status if no match between the healthcare provider information in the first healthcare provider enrollment verification request and the healthcare provider information in the drug distribution management database is identified; (iv) generating, by the one or more processors, a first patient enrollment status, comprising (1) accessing the drug distribution management database, and (2) comparing the patient information in the first patient enrollment verification request to enrolled patient information in the drug distribution management database, and (3) obtaining a positive patient enrollment status by identifying a match between the patient information in the first patient enrollment verification request and the patient information in the drug distribution management database, or obtaining a negative patient enrollment status if no match between the patient information in the first patient enrollment verification request and the patient information in the drug distribution management database is identified; and (v) sending, to the second electronic device, the first healthcare setting enrollment status, the first healthcare provider enrollment status, and the first patient enrollment status, wherein receipt of a positive first healthcare setting enrollment status, a positive first healthcare provider enrollment status, and a positive first patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule; (b) authorizing, by the one or more processors, a plurality of doses of the drug at a single treatment dose level according to an oral immunotherapy dosing schedule, comprising: (i) receiving, by the one or more processors and from the second electronic device, a second healthcare provider enrollment verification request comprising healthcare provider information for a healthcare provider and a second patient enrollment verification request comprising patient information for a patient; (ii) generating, by the one or more processors, a second healthcare provider enrollment status, comprising (1) accessing the drug distribution management database, (2) comparing the healthcare provider information in the second healthcare provider enrollment verification request to enrolled healthcare provider information in the drug distribution management database, and (3) obtaining a positive healthcare provider enrollment status by identifying a match between the healthcare provider information in the second healthcare provider enrollment verification request and the healthcare provider information in the drug distribution management database, or obtaining a negative healthcare provider enrollment status if no match between the healthcare provider information in the second healthcare provider enrollment verification request and the healthcare provider information in the drug distribution management database is identified; (iii) generating, by the one or more processors, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare provider enrollment status and the second patient enrollment status, wherein receipt of a positive second healthcare provider enrollment status and a positive second patient enrollment status by the pharmacy authorizes the pharmacy to provide to the healthcare setting, the healthcare provider, or the patient a plurality of doses of the drug at a single treatment dose level according to an oral immunotherapy dosing schedule; (c) repeating step (b) at a different treatment dose level, wherein the pharmacy is not authorized to simultaneously provide the drug at more than one treatment dose level for the patient; (d) authorizing, by the one or more processors, the provision of an oral immunotherapy treatment kit comprising a plurality of doses of a drug for treating a food allergy at different up-dosing levels to a healthcare setting, comprising: (i) receiving, by the one or more processors and from a second electronic device associated with a distributor enrolled in the drug distribution management database, a second healthcare setting enrollment verification request comprising healthcare setting information for a healthcare setting; (ii) generating, by the one or more processors, a second healthcare setting enrollment status, comprising (1) accessing the drug distribution management database, (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; and (iii) sending, to the third electronic device, the second healthcare setting enrollment status, wherein receipt of a positive second healthcare setting enrollment status authorizes the distributor to provide the oral immunotherapy treatment kit to the healthcare setting.

In some embodiments, a non-transitory computer-readable storage medium storing one or more programs comprising instructions, which when executed by one or more processors of an electronic device, cause the electronic device to: (a) receive, from a second electronic device associated with a pharmacy enrolled in a drug distribution management database for a drug comprising a food allergen, a healthcare setting enrollment verification request comprising healthcare setting information for a healthcare setting and a patient enrollment verification request comprising patient information for a patient; (b) generate, by the one or more processors, a healthcare setting enrollment status by (i) accessing the drug distribution management database, (ii) comparing the healthcare setting information in the healthcare setting enrollment verification request to the healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generate, by the one or more processors, a patient enrollment status by (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) send, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule.

In some embodiments, a non-transitory computer-readable storage medium storing one or more programs comprising instructions, which when executed by one or more processors of an electronic device, cause the electronic device to: (a) receive, from a second electronic device associated with a pharmacy enrolled in a drug distribution management database for a drug comprising a food allergen, a healthcare setting enrollment verification request comprising healthcare setting information for a healthcare setting, a healthcare provider enrollment verification request comprising healthcare provider information for a healthcare provider, and a patient enrollment verification request comprising patient information for a patient; (b) generate, by the one or more processors, a healthcare setting enrollment status by (i) accessing the drug distribution management database, (ii) comparing the healthcare setting information in the healthcare setting enrollment verification request to the healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generate, by the one or more processors, a healthcare provider enrollment status by (i) accessing the drug distribution management database, (ii) comparing the healthcare provider information in the healthcare provider enrollment verification request to the healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; (d) generate, by the one or more processors, a patient enrollment status by (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) send, to the second electronic device, the healthcare setting enrollment status, the healthcare provider enrollment status, and the patient enrollment status, wherein receipt of a positive healthcare setting enrollment status, a positive healthcare provider enrollment status, and a positive patient enrollment status by the pharmacy authorizes the pharmacy to provide to the healthcare setting or the healthcare provider an initial dose escalation kit associated with the patient, the initial dose escalation kit comprising a plurality of different doses of the drug according to an initial dose escalation schedule.

In some embodiments, a non-transitory computer-readable storage medium storing one or more programs comprising instructions, which when executed by one or more processors of an electronic device, cause the electronic device to: (a) receive, from a second electronic device associated with a pharmacy enrolled in a drug distribution management database for a drug comprising a food allergen, a healthcare setting enrollment verification request comprising healthcare setting information for a healthcare setting and a patient enrollment verification request comprising patient information for a patient; (b) generate, by the one or more processors, a healthcare setting enrollment status by (i) accessing the drug distribution management database, (ii) comparing the healthcare setting information in the healthcare setting enrollment verification request to the healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generate, by the one or more processors, a patient enrollment status by (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) send, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting or the patient a plurality of doses of the drug at a single treatment dose level according to an oral immunotherapy dosing schedule. In some embodiments, the instructions, which when executed by the one or more processors of the electronic device, further cause the electronic device to repeat steps (a)-(d) at a plurality of different treatment dose levels, wherein the pharmacy is not authorized to simultaneously provide the drug at more than one treatment dose level for the patient.

In some embodiments, a non-transitory computer-readable storage medium storing one or more programs comprising instructions, which when executed by one or more processors of an electronic device, cause the electronic device to: (a) receive, from a second electronic device associated with a pharmacy enrolled in a drug distribution management database for a drug comprising a food allergen, a healthcare provider enrollment verification request comprising healthcare provider information for a healthcare provider and a patient enrollment verification request comprising patient information for a patient; (b) generate, by the one or more processors, a healthcare provider enrollment status by (i) accessing the drug distribution management database, (ii) comparing the healthcare provider information in the healthcare provider enrollment verification request to the healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; (c) generate, by the one or more processors, a patient enrollment status by (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) send, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting, healthcare provider, or the patient a plurality of doses of the drug at a single treatment dose level according to an oral immunotherapy dosing schedule. In some embodiments, the instructions, which when executed by the one or more processors of the electronic device, further cause the electronic device to repeat steps (a)-(d) at a plurality of different treatment dose levels, wherein the pharmacy is not authorized to simultaneously provide the drug at more than one treatment dose level for the patient.

In some embodiments of any one of the storage media described above, the instructions, which when executed by the one or more processors of the electronic device, further cause the electronic device to: receive, by the one or more processors, a patient enrollment request comprising patient information for the patient; and add, by the one or more processors, an entry comprising the patient information into the drug distribution management database to enroll the patient in the drug distribution management database.

In some embodiments of any one of the storage media described above, the instructions, which when executed by the one or more processors of the electronic device, further cause the electronic device to: receive, by the one or more processors, a pharmacy enrollment request comprising pharmacy information for the pharmacy; and add, by the one or more processors, an entry comprising the pharmacy information to the drug distribution management database to enroll the pharmacy in the drug distribution management database.

In some embodiments of any one of the storage media described above, the instructions, which when executed by the one or more processors of the electronic device, further cause the electronic device to: receive, by the one or more processors, a healthcare provider enrollment request comprising healthcare provider information for the healthcare provider; and add, by the one or more processors, an entry comprising the healthcare provider information to the drug distribution management database to enroll the healthcare provider in the drug distribution management database.

In some embodiments of any one of the storage media described above, the instructions, which when executed by the one or more processors of the electronic device, further cause the electronic device to: receive, by the one or more processors, a healthcare setting enrollment request comprising healthcare setting information for the healthcare setting; and add, by the one or more processors, an entry comprising the healthcare setting information to the drug distribution management database to enroll the healthcare setting in the drug distribution management database.

In some embodiments, a non-transitory computer-readable storage medium storing one or more programs comprising instructions, which when executed by one or more processors of an electronic device, cause the electronic device to: (a) receive, by the one or more processors, a pharmacy enrollment verification request comprising pharmacy information for a pharmacy; (b) generate by the one or more processors, a pharmacy enrollment status by (i) accessing a drug distribution management database for a drug comprising a food allergen, (ii) comparing the pharmacy information in the pharmacy enrollment verification request to enrolled pharmacy information in the drug distribution management database, and (iii) obtaining a positive pharmacy enrollment status by identifying a match between the pharmacy information in the pharmacy enrollment verification request and the pharmacy information in the drug distribution management database, or obtaining a negative pharmacy enrollment status if no match between the pharmacy information in the pharmacy enrollment verification request and the pharmacy information in the drug distribution management database is identified; and (c) send, to a second electronic device associated with the distributor, the pharmacy enrollment status, wherein receipt of a positive pharmacy enrollment status authorizes the distributor to provide the drug to the pharmacy. In some embodiments, the instructions, which when executed by the one or more processors of the electronic device, further cause the electronic device to: receive, by the one or more processors, a pharmacy enrollment request comprising pharmacy information for the pharmacy; and add, by the one or more processors, an entry comprising the pharmacy information into the drug distribution management database to enroll the pharmacy in the drug distribution management database. In some embodiments, the instructions, which when executed by the one or more processors of the electronic device, further cause the electronic device to: receive, by the one or more processors, a distributor enrollment request comprising distributor information for the distributor; and add, by the one or more processors, an entry comprising the distributor information into the drug distribution management database to enroll the distributor in the drug distribution management database.

In some embodiments, a non-transitory computer-readable storage medium storing one or more programs comprising instructions, which when executed by one or more processors of an electronic device, cause the electronic device to: (a) receive, by the one or more processors, a healthcare setting enrollment verification request comprising healthcare setting information for a healthcare setting; (b) generate, by the one or more processors, a healthcare setting enrollment status by (i) accessing a drug distribution management database for a drug comprising a food allergen, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; and (c) send, to a second electronic device associated with the distributor, the healthcare setting enrollment status, wherein receipt of a positive healthcare setting enrollment status authorizes the distributor to provide the oral immunotherapy treatment kit to the healthcare setting. In some embodiments, the instructions, which when executed by the one or more processors of the electronic device, further cause the electronic device to: receive, by the one or more processors, a healthcare setting enrollment request comprising healthcare setting information for the healthcare setting; and add, by the one or more processors, an entry comprising the healthcare setting information into the drug distribution management database to enroll the healthcare setting in the drug distribution management database. In some embodiments, the instructions, which when executed by the one or more processors of the electronic device, further cause the electronic device to: receive, by the one or more processors, a distributor enrollment request comprising distributor information for the distributor; and add, by the one or more processors, an entry comprising the distributor information into the drug distribution management database to enroll the distributor in the drug distribution management database.

In some embodiments, a non-transitory computer-readable storage medium storing one or more programs comprising instructions, which when executed by one or more processors of an electronic device, cause the electronic device to: (a) authorize, by the one or more processors, the provision of an initial dose escalation kit comprising plurality of different doses of the drug comprising a food allergen according to an initial dose escalation schedule by: (i) receiving, by the one or more processors and from a second electronic device associated with a pharmacy enrolled in the drug distribution management database, a first healthcare setting enrollment verification request comprising healthcare setting information for a healthcare setting and a first patient enrollment verification request comprising patient information for a patient; (ii) generating, by the one or more processors, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the first healthcare setting enrollment verification request and the healthcare setting information in the drug distribution management database, or obtaining a negative healthcare setting enrollment status if no match between the healthcare setting information in the first healthcare setting enrollment verification request and the healthcare setting information in the drug distribution management database is identified; (iii) generating, by the one or more processors, a first patient enrollment status, comprising (1) accessing the drug distribution management database, and (2) comparing the patient information in the first patient enrollment verification request to enrolled patient information in the drug distribution management database, and (3) obtaining a positive patient enrollment status by identifying a match between the patient information in the first patient enrollment verification request and the patient information in the drug distribution management database, or obtaining a negative patient enrollment status if no match between the patient information in the first patient enrollment verification request and the patient information in the drug distribution management database is identified; and (iv) sending, to the second electronic device, the first healthcare setting enrollment status and the first patient enrollment status, wherein receipt of a positive first healthcare setting enrollment status and a positive first patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule; (b) authorize, by the one or more processors, a plurality of doses of the drug at a single treatment dose level according to an oral immunotherapy dosing schedule by: (i) receiving, by the one or more processors and from the second electronic device, a second healthcare setting enrollment verification request comprising healthcare setting information for a healthcare setting and a second patient enrollment verification request comprising patient information for a patient; (ii) generating, by the one or more processors, a second healthcare setting enrollment status, comprising (1) accessing the drug distribution management database, (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; (iii) generating, by the one or more processors, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare setting enrollment status and the second patient enrollment status, wherein receipt of a positive second healthcare setting enrollment status and a positive second patient enrollment status by the pharmacy authorizes the pharmacy to provide to the healthcare setting or the patient a plurality of doses of the drug at a single treatment dose level according to an oral immunotherapy dosing schedule; (c) repeat step (b) at a different treatment dose level, wherein the pharmacy is not authorized to simultaneously provide the drug at more than one treatment dose level for the patient; (d) authorize, by the one or more processors, the provision of an oral immunotherapy treatment kit comprising a plurality of doses of a drug for treating a food allergy at different up-dosing levels to a healthcare setting, by: (i) receiving, by the one or more processors and from a third electronic device associated with a distributor enrolled in the drug distribution management database, a third healthcare setting enrollment verification request comprising healthcare setting information for a healthcare setting; (ii) generating, by the one or more processors, a third healthcare setting enrollment status, comprising (1) accessing the drug distribution management database, (2) comparing the healthcare setting information in the third healthcare setting enrollment verification request to enrolled healthcare setting information in the drug distribution management database, and (3) obtaining a positive healthcare setting enrollment status by identifying a match between the healthcare setting information in the third healthcare setting enrollment verification request and the healthcare setting information in the drug distribution management database, or obtaining a negative healthcare setting enrollment status if no match between the healthcare setting information in the third healthcare setting enrollment verification request and the healthcare setting information in the drug distribution management database is identified; and (iii) sending, to the third electronic device, the third healthcare setting enrollment status, wherein receipt of a positive third healthcare setting enrollment status authorizes the distributor to provide the oral immunotherapy treatment kit to the healthcare setting. In some embodiments, the instructions, which when executed by the one or more processors of the electronic device, further cause the electronic device to: receive, by the one or more processors, a pharmacy enrollment request comprising pharmacy information for the pharmacy; and add, by the one or more processors, an entry comprising the pharmacy information into the drug distribution management database to enroll the pharmacy in the drug distribution management database. In some embodiments, the instructions, which when executed by the one or more processors of the electronic device, further cause the electronic device to: receive, by the one or more processors, a distributor enrollment request comprising distributor information for the distributor; and add, by the one or more processors, an entry comprising the distributor information into the drug distribution management database to enroll the distributor in the drug distribution management database. In some embodiments, the instructions, which when executed by the one or more processors of the electronic device, further cause the electronic device to: receive, by the one or more processors, a patient enrollment request comprising patient information for the patient; and add, by the one or more processors, an entry comprising the patient information into the drug distribution management database to enroll the patient in the drug distribution management database. In some embodiments, the instructions, which when executed by the one or more processors of the electronic device, further cause the electronic device to: receive, by the one or more processors, a healthcare setting enrollment request comprising healthcare setting information for the healthcare setting; and add, by the one or more processors, an entry comprising the healthcare setting information into the drug distribution management database to enroll the healthcare setting in the drug distribution management database.

In some embodiments, a non-transitory computer-readable storage medium storing one or more programs comprising instructions, which when executed by one or more processors of an electronic device, cause the electronic device to: (a) authorize, by the one or more processors, the provision of an initial dose escalation kit comprising plurality of different doses of the drug comprising a food allergen according to an initial dose escalation schedule by: (i) receiving, by the one or more processors and from a second electronic device associated with a pharmacy enrolled in the drug distribution management database, a first healthcare setting enrollment verification request comprising healthcare setting information for a healthcare setting, a first healthcare provider enrollment verification request comprising healthcare provider information for a healthcare provider, and a first patient enrollment verification request comprising patient information for a patient; (ii) generating, by the one or more processors, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the first healthcare setting enrollment verification request and the healthcare setting information in the drug distribution management database, or obtaining a negative healthcare setting enrollment status if no match between the healthcare setting information in the first healthcare setting enrollment verification request and the healthcare setting information in the drug distribution management database is identified; (iii) generating, by the one or more processors, a first healthcare provider enrollment status, comprising (1) accessing the drug distribution management database, (2) comparing the healthcare provider information in the first healthcare provider enrollment verification request to enrolled healthcare provider information in the drug distribution management database, and (3) obtaining a positive healthcare provider enrollment status by identifying a match between the healthcare provider information in the first healthcare provider enrollment verification request and the healthcare provider information in the drug distribution management database, or obtaining a negative healthcare provider enrollment status if no match between the healthcare provider information in the first healthcare provider enrollment verification request and the healthcare provider information in the drug distribution management database is identified; (iv) generating, by the one or more processors, a first patient enrollment status, comprising (1) accessing the drug distribution management database, and (2) comparing the patient information in the first patient enrollment verification request to enrolled patient information in the drug distribution management database, and (3) obtaining a positive patient enrollment status by identifying a match between the patient information in the first patient enrollment verification request and the patient information in the drug distribution management database, or obtaining a negative patient enrollment status if no match between the patient information in the first patient enrollment verification request and the patient information in the drug distribution management database is identified; and (v) sending, to the second electronic device, the first healthcare setting enrollment status, the first healthcare provider enrollment status, and the first patient enrollment status, wherein receipt of a positive first healthcare setting enrollment status, a positive healthcare provider enrollment status, and a positive first patient enrollment status by the pharmacy authorizes the pharmacy to provide to the healthcare setting or healthcare provider an initial dose escalation kit associated with the patient, the initial dose escalation kit comprising a plurality of different doses of the drug according to an initial dose escalation schedule; (b) authorize, by the one or more processors, a plurality of doses of the drug at a single treatment dose level according to an oral immunotherapy dosing schedule by: (i) receiving, by the one or more processors and from the second electronic device, a second healthcare provider enrollment verification request comprising healthcare provider information for a healthcare provider and a second patient enrollment verification request comprising patient information for a patient; (ii) generating, by the one or more processors, a second healthcare provider enrollment status, comprising (1) accessing the drug distribution management database, (2) comparing the healthcare provider information in the second healthcare provider enrollment verification request to enrolled healthcare provider information in the drug distribution management database, and (3) obtaining a positive healthcare provider enrollment status by identifying a match between the healthcare provider information in the second healthcare provider enrollment verification request and the healthcare provider information in the drug distribution management database, or obtaining a negative healthcare provider enrollment status if no match between the healthcare provider information in the second healthcare provider enrollment verification request and the healthcare provider information in the drug distribution management database is identified; (iii) generating, by the one or more processors, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare provider enrollment status and the second patient enrollment status, wherein receipt of a positive second healthcare provider enrollment status and a positive second patient enrollment status by the pharmacy authorizes the pharmacy to provide to the healthcare setting, healthcare provider, or the patient a plurality of doses of the drug at a single treatment dose level according to an oral immunotherapy dosing schedule; (c) repeat step (b) at a different treatment dose level, wherein the pharmacy is not authorized to simultaneously provide the drug at more than one treatment dose level for the patient; (d) authorize, by the one or more processors, the provision of an oral immunotherapy treatment kit comprising a plurality of doses of a drug for treating a food allergy at different up-dosing levels to a healthcare setting, by: (i) receiving, by the one or more processors and from a third electronic device associated with a distributor enrolled in the drug distribution management database, a second healthcare setting enrollment verification request comprising healthcare setting information for a healthcare setting; (ii) generating, by the one or more processors, a second healthcare setting enrollment status, comprising (1) accessing the drug distribution management database, (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; and (iii) sending, to the third electronic device, the second healthcare setting enrollment status, wherein receipt of a positive second healthcare setting enrollment status authorizes the distributor to provide the oral immunotherapy treatment kit to the healthcare setting. In some embodiments, the instructions, which when executed by the one or more processors of the electronic device, further cause the electronic device to: receive, by the one or more processors, a pharmacy enrollment request comprising pharmacy information for the pharmacy; and add, by the one or more processors, an entry comprising the pharmacy information into the drug distribution management database to enroll the pharmacy in the drug distribution management database. In some embodiments, the instructions, which when executed by the one or more processors of the electronic device, further cause the electronic device to: receive, by the one or more processors, a distributor enrollment request comprising distributor information for the distributor; and add, by the one or more processors, an entry comprising the distributor information into the drug distribution management database to enroll the distributor in the drug distribution management database. In some embodiments, the instructions, which when executed by the one or more processors of the electronic device, further cause the electronic device to: receive, by the one or more processors, a patient enrollment request comprising patient information for the patient; and add, by the one or more processors, an entry comprising the patient information into the drug distribution management database to enroll the patient in the drug distribution management database. In some embodiments, the instructions, which when executed by the one or more processors of the electronic device, further cause the electronic device to: receive, by the one or more processors, a healthcare provider enrollment request comprising healthcare provider information for the healthcare provider; and add, by the one or more processors, an entry comprising the healthcare provider information into the drug distribution management database to enroll the healthcare provider in the drug distribution management database. In some embodiments, the instructions, which when executed by the one or more processors of the electronic device, further cause the electronic device to: receive, by the one or more processors, a healthcare setting enrollment request comprising healthcare setting information for the healthcare setting; and add, by the one or more processors, an entry comprising the healthcare setting information into the drug distribution management database to enroll the healthcare setting in the drug distribution management database.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

### EXEMPLARY EMBODIMENTS

The following embodiments are exemplary and not intended to limit the scope of the invention described herein.

Embodiment 1. A computer-implemented method for authorizing the provision of a drug comprising a food allergen to a healthcare setting, comprising: (a) receiving, at a first electronic device communicatively coupled to a drug distribution management database for the drug and from a second electronic device associated with a pharmacy enrolled in the drug distribution management database, a healthcare setting enrollment verification request comprising healthcare setting information for a healthcare setting and a patient enrollment verification request comprising patient information for a patient; (b) generating, at the first electronic device, a healthcare setting enrollment status, comprising (i) accessing the drug distribution management database, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generating, at the first electronic device, a patient enrollment status, comprising (i) accessing the drug distribution management database, (ii) comparing the patient information in the patient enrollment verification request to enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule.

Embodiment 2. The method of embodiment 1, wherein step (a) further comprises receiving, at the first electronic device, from the second electronic device, a healthcare provider enrollment verification request comprising healthcare provider information for a healthcare provider; wherein the method further comprises a step of generating, at the first electronic device, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; wherein step (d) further comprises sending, to the second electronic device, the healthcare provider enrollment status, wherein receipt of each of a positive healthcare setting enrollment status, a positive patient enrollment status, and a positive healthcare provider enrollment status by the pharmacy authorizes the pharmacy to provide the healthcare setting the initial dose escalation kit associated with the patient.

Embodiment 3. A computer-implemented method for authorizing the provision of a drug comprising a food allergen to a healthcare setting or a patient, comprising: (a) receiving, at a first electronic device communicatively coupled to a drug distribution management database for the drug and from a second electronic device associated with a pharmacy enrolled in the drug distribution management database, a healthcare setting enrollment verification request comprising healthcare setting information for a healthcare setting and a patient enrollment verification request comprising patient information for a patient; (b) generating, at the first electronic device, a healthcare setting enrollment status, comprising (i) accessing the drug distribution management database, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generating, at the first electronic device, a patient enrollment status, comprising (i) accessing the drug distribution management database, (ii) comparing the patient information in the patient enrollment verification request to enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting or the patient a plurality of doses of the drug at a single treatment dose level according to an oral immunotherapy dosing schedule.

Embodiment 4. The method of embodiment 3, wherein step (a) further comprises receiving, at the first electronic device, from the second electronic device, a healthcare provider enrollment verification request comprising healthcare provider information for a healthcare provider; wherein the method further comprises generating, at the first electronic device, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; wherein step (d) further comprises sending, to the second electronic device, the healthcare provider enrollment status, wherein receipt of each of a positive healthcare setting enrollment status, a positive patient enrollment status, and a positive healthcare provider enrollment status by the pharmacy authorizes the pharmacy to provide to the healthcare setting or the patient the plurality of doses of the drug at a single treatment dose level.

Embodiment 5. A computer-implemented method for authorizing the provision of a drug comprising a food allergen to a patient, comprising: (a) receiving, at a first electronic device communicatively coupled to a drug distribution management database for the drug and from a second electronic device associated with a pharmacy enrolled in the drug distribution management database, a healthcare provider enrollment verification request comprising healthcare provider information for a healthcare provider and a patient enrollment verification request comprising patient information for a patient; (b) generating, at the first electronic device, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; (c) generating, at the first electronic device, a patient enrollment status, comprising (i) accessing the drug distribution management database, (ii) comparing the patient information in the patient enrollment verification request to enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare provider enrollment status and the patient enrollment status, wherein receipt of a positive healthcare provider enrollment status and a positive patient enrollment status by the pharmacy authorizes the pharmacy to provide to the healthcare provider or the patient a plurality of doses of the drug at a single treatment dose level according to an oral immunotherapy dosing schedule.

Embodiment 6. The method of any one of embodiments 3-5, further comprising repeating steps (a)-(d) at a plurality of different treatment dose levels, wherein the pharmacy is not authorized to simultaneously provide the drug at more than one treatment dose level for the patient.

Embodiment 7. The method of any one of embodiments 1-6, further comprising: receiving, by the first electronic device, a patient enrollment request comprising patient information for the patient; and adding, at the first electronic device, an entry comprising the patient information into the drug distribution management database to enroll the patient in the drug distribution management database.

Embodiment 8. The method of any one of embodiments 1-7, further comprising: receiving, at the first electronic device, a pharmacy enrollment request comprising pharmacy information for the pharmacy; and adding, at the first electronic device, an entry comprising the pharmacy information to the drug distribution management database to enroll the pharmacy in the drug distribution management database.

Embodiment 9. The method of any one of embodiments 1-8, further comprising: receiving, at the first electronic device, a healthcare setting enrollment request comprising healthcare setting information for the healthcare setting; and adding, at the first electronic device, an entry comprising the healthcare setting information to the drug distribution management database to enroll the healthcare setting in the drug distribution management database.

Embodiment 10. The method of any one of embodiments 2 or 4-9, further comprising: receiving, at the first electronic device, a healthcare provider enrollment request comprising healthcare provider information for the healthcare provider; and adding, at the first electronic device, an entry comprising the healthcare provider information to the drug distribution management database to enroll the healthcare provider in the drug distribution management database.

Embodiment 11. A computer program product comprising instructions which, when the program is executed by a computer, causes the computer to carry out the steps of the method of any one of embodiments 1-10.

Embodiment 12. An electronic device comprising: one or more processors; a memory configured to store a drug distribution management database for a drug comprising a food allergen, the drug distribution management database comprising entries for enrolled patients comprising enrolled patient information, entries for enrolled healthcare settings comprising enrolled healthcare setting information, and entries for enrolled pharmacies comprising enrolled pharmacy information; and one or more programs stored on the memory configured to be executed by the one or more processors, the one or more programs including instructions for: (a) receiving, from a second electronic device associated with a pharmacy enrolled in the drug distribution management database, a healthcare setting enrollment verification request comprising healthcare setting information for a healthcare setting and a patient enrollment verification request comprising patient information for a patient; (b) generating, by the one or more processors, a healthcare setting enrollment status, comprising (i) accessing the drug distribution management database, (ii) comparing the healthcare setting information in the healthcare setting enrollment verification request to the healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generating, by the one or more processors, a patient enrollment status, comprising (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule.

Embodiment 13. The electronic device of embodiment 12: wherein the drug distribution management database further comprises entries for enrolled healthcare providers comprising enrolled healthcare provider information; wherein the instructions of (a) further comprises receiving, at the first electronic device, from the second electronic device, a healthcare provider enrollment verification request comprising healthcare provider information for a healthcare provider; wherein the one or more programs further include instructions for generating, at the first electronic device, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; wherein the instructions of (d) further comprise sending, to the second electronic device, the healthcare provider enrollment status, wherein receipt of each of a positive healthcare setting enrollment status, a positive patient enrollment status, and a positive healthcare provider enrollment status by the pharmacy authorizes the pharmacy to provide the healthcare setting the initial dose escalation kit associated with the patient.

Embodiment 14. An electronic device comprising: one or more processors; a memory configured to store a drug distribution management database for a drug comprising a food allergen, the drug distribution management database comprising entries for enrolled patients comprising enrolled patient information, entries for enrolled healthcare providers comprising enrolled healthcare provider information, and entries for enrolled pharmacies comprising enrolled pharmacy information; and one or more programs stored on the memory configured to be executed by the one or more processors, the one or more programs including instructions for: (a) receiving, from a second electronic device associated with a pharmacy enrolled in the drug distribution management database, a healthcare provider enrollment verification request comprising healthcare provider information for a healthcare provider and a patient enrollment verification request comprising patient information for a patient; (b) generating, at the first electronic device, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; (c) generating, at the first electronic device, a patient enrollment status, comprising (i) accessing the drug distribution management database, (ii) comparing the patient information in the patient enrollment verification request to enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare provider enrollment status and the patient enrollment status, wherein receipt of a positive healthcare provider enrollment status and a positive patient enrollment status by the pharmacy authorizes the pharmacy to provide to the healthcare provider or the patient a plurality of doses of the drug at a single treatment dose level according to an oral immunotherapy dosing schedule.

Embodiment 15. An electronic device comprising: one or more processors; a memory configured to store a drug distribution management database for a drug comprising a food allergen, the drug distribution management database comprising entries for enrolled patients comprising enrolled patient information, entries for enrolled healthcare settings comprising enrolled healthcare setting information, and entries for enrolled pharmacies comprising enrolled pharmacy information; and one or more programs stored on the memory configured to be executed by the one or more processors, the one or more programs including instructions for: (a) receiving, from a second electronic device associated with a pharmacy enrolled in the drug distribution management database, a healthcare setting enrollment verification request comprising healthcare setting information for a healthcare setting and a patient enrollment verification request comprising patient information for a patient; (b) generating, by the one or more processors, a healthcare setting enrollment status, comprising (i) accessing the drug distribution management database, (ii) comparing the healthcare setting information in the healthcare setting enrollment verification request to the healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generating, by the one or more processors, a patient enrollment status, comprising (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) sending, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting or the patient a plurality of doses of the drug at a single treatment dose level according to an oral immunotherapy dosing schedule.

Embodiment 16. The electronic device of embodiment 15: wherein the drug distribution management database further comprises entries for enrolled healthcare providers comprising enrolled healthcare provider information; wherein the instructions of (a) further comprise receiving, from the second electronic device associated with the pharmacy enrolled in the drug distribution management database, a healthcare provider enrollment verification request comprising healthcare provider information for a healthcare provider; wherein the one or more programs further include instructions for generating, by the one or more processors, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; wherein the instructions of (d) further comprise sending, to the second electronic device, the healthcare provider enrollment status, wherein receipt of each of a positive healthcare setting enrollment status, a positive patient enrollment status, and a positive healthcare provider enrollment status by the pharmacy authorizes the pharmacy to provide to the healthcare setting, the healthcare provider, or the patient the plurality of doses of the drug at the single treatment dose level.

Embodiment 17. The electronic device of embodiment 15 or embodiment 16, the one or more programs further including instructions for repeating steps (a)-(d) at a plurality of different treatment dose levels, wherein the pharmacy is not authorized to simultaneously provide the drug at more than one treatment dose level for the patient.

Embodiment 18. The electronic device of any one of embodiments 12-17, the one or more programs further including instructions for: receiving, by the one or more processors, a patient enrollment request comprising patient information for the patient; and adding, by the one or more processors, an entry comprising the patient information into the drug distribution management database to enroll the patient in the drug distribution management database.

Embodiment 19. The electronic device of any one of embodiments 12-18, the one or more programs further including instructions for: receiving, by the one or more processors, a pharmacy enrollment request comprising pharmacy information for the pharmacy; and adding, by the one or more processors, an entry comprising the pharmacy information to the drug distribution management database to enroll the pharmacy in the drug distribution management database.

Embodiment 20. The electronic device of any one of embodiments 12-19, the one or more programs further including instructions for: receiving, by the one or more processors, a healthcare setting enrollment request comprising healthcare setting information for the healthcare setting; and adding, by the one or more processors, an entry comprising the healthcare setting information to the drug distribution management database to enroll the healthcare setting in the drug distribution management database.

Embodiment 21. The electronic device of any one of embodiments 13-14 or 16-20, the one or more programs further including instructions for: receiving, by the one or more processors, a healthcare provider enrollment request comprising healthcare provider information for the healthcare provider; and adding, by the one or more processors, an entry comprising the healthcare provider information to the drug distribution management database to enroll the healthcare provider in the drug distribution management database.

Embodiment 22. A non-transitory computer-readable storage medium storing one or more programs comprising instructions, which when executed by one or more processors of an electronic device, cause the electronic device to: (a) receive, from a second electronic device associated with a pharmacy enrolled in a drug distribution management database for a drug comprising a food allergen, a healthcare setting enrollment verification request comprising healthcare setting information for a healthcare setting and a patient enrollment verification request comprising patient information for a patient; (b) generate, by the one or more processors, a healthcare setting enrollment status by (i) accessing the drug distribution management database, (ii) comparing the healthcare setting information in the healthcare setting enrollment verification request to the healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generate, by the one or more processors, a patient enrollment status by (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) send, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule.

Embodiment 23. The storage medium of embodiment 22, wherein step (a) further comprises receiving, from the second electronic device, a healthcare provider enrollment verification request comprising healthcare provider information for a healthcare provider; wherein the instructions, which when executed by the one or more processors of the electronic device, further cause the electronic device to generate, by the one or more processors, the healthcare provider enrollment status by (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; wherein step (d) further comprises sending, to the second electronic device, the healthcare provider enrollment status, wherein receipt of each of a positive healthcare setting enrollment status, a positive patient enrollment status, and a positive healthcare provider enrollment status by the pharmacy authorizes the pharmacy to provide to the healthcare setting the initial dose escalation kit associated with the patient.

Embodiment 24. A non-transitory computer-readable storage medium storing one or more programs comprising instructions, which when executed by one or more processors of an electronic device, cause the electronic device to: (a) receive, from a second electronic device associated with a pharmacy enrolled in a drug distribution management database for a drug comprising a food allergen, a healthcare provider enrollment verification request comprising healthcare provider information for a healthcare provider and a patient enrollment verification request comprising patient information for a patient; (b) generate, by the one or more processors, a healthcare provider enrollment status by (i) accessing the drug distribution management database, (ii) comparing the healthcare provider information in the healthcare provider enrollment verification request to the healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; (c) generate, by the one or more processors, a patient enrollment status by (i) accessing the drug distribution management database, (ii) comparing the patient information in the patient enrollment verification request to the enrolled patient information in the drug distribution management database, and (iii) obtaining a positive patient enrollment status by identifying a match between the patient information in the healthcare setting enrollment verification request and the patient information in the drug distribution management database, or obtaining a negative patient enrollment status if no match between the patient information in the patient enrollment verification request and the patient information in the drug distribution management database is identified; and (d) send, to the second electronic device, the healthcare provider enrollment status and the patient enrollment status, wherein receipt of a positive healthcare provider enrollment status and a positive patient enrollment status by the pharmacy authorizes the pharmacy to provide to the healthcare provider or the patient a plurality of doses of the drug at a single treatment dose level according to an oral immunotherapy dosing schedule.

Embodiment 25. A non-transitory computer-readable storage medium storing one or more programs comprising instructions, which when executed by one or more processors of an electronic device, cause the electronic device to: (a) receive, from a second electronic device associated with a pharmacy enrolled in a drug distribution management database for a drug comprising a food allergen, a healthcare setting enrollment verification request comprising healthcare setting information for a healthcare setting and a patient enrollment verification request comprising patient information for a patient; (b) generate, by the one or more processors, a healthcare setting enrollment status by (i) accessing the drug distribution management database, (ii) comparing the healthcare setting information in the healthcare setting enrollment verification request to the healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (c) generate, by the one or more processors, a patient enrollment status by (i) accessing the drug distribution management database, (ii) comparing the patient information in the patient enrollment verification request to the enrolled patient information in the drug distribution management database, and (iii) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (d) send, to the second electronic device, the healthcare setting enrollment status and the patient enrollment status, wherein receipt of a positive healthcare setting enrollment status and a positive patient enrollment status by the pharmacy authorizes the pharmacy to provide to the healthcare setting or the patient a plurality of doses of the drug at a single treatment dose level according to an oral immunotherapy dosing schedule.

Embodiment 26. The storage medium of embodiment 25: wherein (a) further comprise receiving, from the second electronic device associated with the pharmacy enrolled in the drug distribution management database, a healthcare provider enrollment verification request comprising healthcare provider information for a healthcare provider; wherein the instructions, which when executed by the one or more processors of the electronic device, further cause the electronic device to generate, by the one or more processors, a healthcare provider enrollment status, comprising (i) accessing the drug distribution management database, (ii) comparing the healthcare provider information in the healthcare provider enrollment verification request to enrolled healthcare provider information in the drug distribution management database, and (iii) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; wherein (d) further comprises sending, to the second electronic device, the healthcare provider enrollment status, wherein receipt of each of a positive healthcare setting enrollment status, a positive patient enrollment status, and a positive healthcare provider enrollment status by the pharmacy authorizes the pharmacy to provide to the healthcare setting, healthcare provider, or the patient the plurality of doses of the drug at the single treatment dose level.

Embodiment 27. The storage medium of embodiment 25 or embodiment 26, wherein the instructions, which when executed by the one or more processors of the electronic device, further cause the electronic device to repeat steps (a)-(d) at a plurality of different treatment dose levels, wherein the pharmacy is not authorized to simultaneously provide the drug at more than one treatment dose level for the patient.

Embodiment 28. The storage medium of any one of embodiments 22-27, wherein the instructions, which when executed by the one or more processors of the electronic device, further cause the electronic device to: receive, by the one or more processors, a patient enrollment request comprising patient information for the patient; and add, by the one or more processors, an entry comprising the patient information into the drug distribution management database to enroll the patient in the drug distribution management database.

Embodiment 29. The storage medium of any one of embodiments 22-28, wherein the instructions, which when executed by the one or more processors of the electronic device, further cause the electronic device to: receive, by the one or more processors, a pharmacy enrollment request comprising pharmacy information for the pharmacy; and add, by the one or more processors, an entry comprising the pharmacy information to the drug distribution management database to enroll the pharmacy in the drug distribution management database.

Embodiment 30. The storage medium of any one of embodiments 22-29, wherein the instructions, which when executed by the one or more processors of the electronic device, further cause the electronic device to: receive, by the one or more processors, a healthcare setting enrollment request comprising healthcare setting information for the healthcare setting; and add, by the one or more processors, an entry comprising the healthcare setting information to the drug distribution management database to enroll the healthcare setting in the drug distribution management database.

Embodiment 31. The storage medium of any one of embodiments 23-24 or 26-30, wherein the instructions, which when executed by the one or more processors of the electronic device, further cause the electronic device to: receive, by the one or more processors, a healthcare provider enrollment request comprising healthcare provider information for the healthcare provider; and add, by the one or more processors, an entry comprising the healthcare provider information to the drug distribution management database to enroll the healthcare provider in the drug distribution management database.

Embodiment 32. A computer-implemented method for authorizing the provision of a drug comprising a food allergen to a pharmacy, comprising: (a) receiving, at a first electronic device communicatively coupled to a drug distribution management database for the drug and from a second electronic device associated with a distributor enrolled in the drug distribution management database, a pharmacy enrollment verification request comprising pharmacy information for a pharmacy; (b) generating, at the first electronic device, a pharmacy enrollment status, comprising (i) accessing the drug distribution management database, (ii) comparing the pharmacy information in the pharmacy enrollment verification request to enrolled pharmacy information in the drug distribution management database, and (iii) obtaining a positive pharmacy enrollment status by identifying a match between the pharmacy information in the pharmacy enrollment verification request and the pharmacy information in the drug distribution management database, or obtaining a negative pharmacy enrollment status if no match between the pharmacy information in the pharmacy enrollment verification request and the pharmacy information in the drug distribution management database is identified; and (c) sending, to the second electronic device, the pharmacy enrollment status, wherein receipt of a positive pharmacy enrollment status authorizes the distributor to provide the drug to the pharmacy.

Embodiment 33. The method of embodiment 32, further comprising: receiving, by the first electronic device, a pharmacy enrollment request comprising pharmacy information for the pharmacy; and adding, at the first electronic device, an entry comprising the pharmacy information into the drug distribution management database to enroll the pharmacy in the drug distribution management database.

Embodiment 34. The method of embodiment 32 or 33, further comprising: receiving, by the first electronic device, a distributor enrollment request comprising distributor information for the distributor; and adding, at the first electronic device, an entry comprising the distributor information into the drug distribution management database to enroll the distributor in the drug distribution management database.

Embodiment 35. A computer program product comprising instructions which, when the program is executed by a computer, causes the computer to carry out the steps of the method of any one of embodiments 32-34.

Embodiment 36. An electronic device comprising: one or more processors; a memory configured to store a drug distribution management database for a drug comprising a food allergen, the drug distribution management database comprising entries for enrolled distributors comprising enrolled distributor information, and entries for enrolled pharmacies comprising enrolled pharmacy information; and one or more programs stored on the memory configured to be executed by the one or more processors, the one or more programs including instructions for: (a) receiving, by the one or more processors, a pharmacy enrollment verification request comprising pharmacy information for a pharmacy; (b) generating, by the one or more processors, a pharmacy enrollment status, comprising (i) accessing the drug distribution management database, (ii) comparing the pharmacy information in the pharmacy enrollment verification request to enrolled pharmacy information in the drug distribution management database, and (iii) obtaining a positive pharmacy enrollment status by identifying a match between the pharmacy information in the pharmacy enrollment verification request and the pharmacy information in the drug distribution management database, or obtaining a negative pharmacy enrollment status if no match between the pharmacy information in the pharmacy enrollment verification request and the pharmacy information in the drug distribution management database is identified; and (c) sending, to a second electronic device associated with the distributor, the pharmacy enrollment status, wherein receipt of a positive pharmacy enrollment status authorizes the distributor to provide the drug to the pharmacy.

Embodiment 37. The electronic device of embodiment 36, the one or more programs further including instructions for: receiving, by the one or more processors, a pharmacy enrollment request comprising pharmacy information for the pharmacy; and adding, by the one or more processors, an entry comprising the pharmacy information into the drug distribution management database to enroll the pharmacy in the drug distribution management database.

Embodiment 38. The electronic device of embodiment 36 or 37, the one or more programs further including instructions for: receiving, by the one or more processors, a distributor enrollment request comprising distributor information for the distributor; and adding, by the one or more processors, an entry comprising the distributor information into the drug distribution management database to enroll the distributor in the drug distribution management database.

Embodiment 39. A non-transitory computer-readable storage medium storing one or more programs comprising instructions, which when executed by one or more processors of an electronic device, cause the electronic device to: (a) receive, by the one or more processors, a pharmacy enrollment verification request comprising pharmacy information for a pharmacy; (b) generate by the one or more processors, a pharmacy enrollment status by (i) accessing a drug distribution management database for a drug comprising a food allergen, (ii) comparing the pharmacy information in the pharmacy enrollment verification request to enrolled pharmacy information in the drug distribution management database, and (iii) obtaining a positive pharmacy enrollment status by identifying a match between the pharmacy information in the pharmacy enrollment verification request and the pharmacy information in the drug distribution management database, or obtaining a negative pharmacy enrollment status if no match between the pharmacy information in the pharmacy enrollment verification request and the pharmacy information in the drug distribution management database is identified; and (c) send, to a second electronic device associated with the distributor, the pharmacy enrollment status, wherein receipt of a positive pharmacy enrollment status authorizes the distributor to provide the drug to the pharmacy.

Embodiment 40. The storage medium of embodiment 39, wherein the instructions, which when executed by the one or more processors of the electronic device, further cause the electronic device to: receive, by the one or more processors, a pharmacy enrollment request comprising pharmacy information for the pharmacy; and add, by the one or more processors, an entry comprising the pharmacy information into the drug distribution management database to enroll the pharmacy in the drug distribution management database.

Embodiment 41. The storage medium of embodiment 39 or 40, wherein the instructions, which when executed by the one or more processors of the electronic device, further cause the electronic device to: receive, by the one or more processors, a distributor enrollment request comprising distributor information for the distributor; and add, by the one or more processors, an entry comprising the distributor information into the drug distribution management database to enroll the distributor in the drug distribution management database.

Embodiment 42. A computer-implemented method for authorizing the distribution of an oral immunotherapy treatment kit comprising a plurality of doses of a drug comprising a food allergen at different dosing levels to a healthcare setting, comprising: (a) receiving, at a first electronic device communicatively coupled to a drug distribution management database for the drug and from a second electronic device associated with a distributor enrolled in the drug distribution management database, a healthcare setting enrollment verification request comprising healthcare setting information for a healthcare setting; (b) generating, at the first electronic device, a healthcare setting enrollment status, comprising (i) accessing the drug distribution management database, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; and (c) sending, to the second electronic device, the healthcare setting enrollment status, wherein receipt of a positive healthcare setting enrollment status authorizes the distributor to provide the oral immunotherapy treatment kit to the healthcare setting.

Embodiment 43. The method of embodiment 42, further comprising: receiving, by the first electronic device, a healthcare setting enrollment request comprising healthcare setting information for the healthcare setting; and adding, at the first electronic device, an entry comprising the healthcare setting information into the drug distribution management database to enroll the healthcare setting in the drug distribution management database.

Embodiment 44. The method of embodiment 42 or 43, further comprising: receiving, by the first electronic device, a distributor enrollment request comprising distributor information for the distributor; and adding, at the first electronic device, an entry comprising the distributor information into the drug distribution management database to enroll the distributor in the drug distribution management database.

Embodiment 45. A computer program product comprising instructions which, when the program is executed by a computer, causes the computer to carry out the steps of the method of any one of embodiments 42-44.

Embodiment 46. An electronic device comprising: one or more processors; a memory configured to store a drug distribution management database for a drug comprising a food allergen, the drug distribution management database comprising entries for enrolled distributors comprising enrolled distributor information, and entries for enrolled healthcare settings comprising enrolled healthcare setting information; and one or more programs stored on the memory configured to be executed by the one or more processors, the one or more programs including instructions for: (a) receiving, by the one or more processors, a healthcare setting enrollment verification request comprising healthcare setting information for a healthcare setting; (b) generating, by the one or more processors, a healthcare setting enrollment status, comprising (i) accessing the drug distribution management database, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; and (c) sending, to a second electronic device associated with the distributor, the healthcare setting enrollment status, wherein receipt of a positive healthcare setting enrollment status authorizes the distributor to provide the oral immunotherapy treatment kit to the healthcare setting.

Embodiment 47. The electronic device of embodiment 46, the one or more programs further including instructions for: receiving, by the one or more processors, a healthcare setting enrollment request comprising healthcare setting information for the healthcare setting; and adding, by the one or more processors, an entry comprising the healthcare setting information into the drug distribution management database to enroll the healthcare setting in the drug distribution management database.

Embodiment 48. The electronic device of embodiment 46 or 47, the one or more programs further including instructions for: receiving, by the one or more processors, a distributor enrollment request comprising distributor information for the distributor; and adding, by the one or more processors, an entry comprising the distributor information into the drug distribution management database to enroll the distributor in the drug distribution management database.

Embodiment 49. A non-transitory computer-readable storage medium storing one or more programs comprising instructions, which when executed by one or more processors of an electronic device, cause the electronic device to: (a) receive, by the one or more processors, a healthcare setting enrollment verification request comprising healthcare setting information for a healthcare setting; (b) generate, by the one or more processors, a healthcare setting enrollment status by (i) accessing a drug distribution management database for a drug comprising a food allergen, (ii) comparing the healthcare setting information in the healthcare setting enrollment verification request to enrolled healthcare setting information in the drug distribution management database, and (iii) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; and (c) send, to a second electronic device associated with the distributor, the healthcare setting enrollment status, wherein receipt of a positive healthcare setting enrollment status authorizes the distributor to provide the oral immunotherapy treatment kit to the healthcare setting.

Embodiment 50. The storage medium of embodiment 49, wherein the instructions, which when executed by the one or more processors of the electronic device, further cause the electronic device to: receive, by the one or more processors, a healthcare setting enrollment request comprising healthcare setting information for the healthcare setting; and add, by the one or more processors, an entry comprising the healthcare setting information into the drug distribution management database to enroll the healthcare setting in the drug distribution management database.

Embodiment 51. The storage medium of embodiment 49 or 50, wherein the instructions, which when executed by the one or more processors of the electronic device, further cause the electronic device to: receive, by the one or more processors, a distributor enrollment request comprising distributor information for the distributor; and add, by the one or more processors, an entry comprising the distributor information into the drug distribution management database to enroll the distributor in the drug distribution management database.

Embodiment 52. A computer-implemented method for managing the distribution of a drug comprising a food allergen, comprising: (a) authorizing the provision of an initial dose escalation kit comprising plurality of different doses of the drug according to an initial dose escalation schedule, comprising: (i) receiving, at a first electronic device communicatively coupled to a drug distribution management database for the drug and from a second electronic device associated with a pharmacy enrolled in the drug distribution management database, a first healthcare setting enrollment verification request comprising healthcare setting information for a healthcare setting and a first patient enrollment verification request comprising patient information for a patient; (ii) generating, at the first electronic device, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (iii) generating, at the first electronic device, a first patient enrollment status, comprising (1) accessing the drug distribution management database, (2) comparing the patient information in the first patient enrollment verification request to enrolled patient information in the drug distribution management database, and (3) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (iv) sending, to the second electronic device, the first healthcare setting enrollment status and the first patient enrollment status, wherein receipt of a positive first healthcare setting enrollment status and a positive first patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule; (b) authorizing the provision of a plurality of doses of the drug at a single treatment dose level according to an oral immunotherapy dosing schedule, comprising: (i) receiving, at the first electronic device from the second electronic device, a second healthcare setting enrollment verification request comprising healthcare setting information for a healthcare setting and a second patient enrollment verification request comprising patient information for a patient; (ii) generating, at the first electronic device, a second healthcare setting enrollment status, comprising (1) accessing the drug distribution management database, (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; (iii) generating, at the first electronic device, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare setting enrollment status and the second patient enrollment status, wherein receipt of a positive second healthcare setting enrollment status and a positive second patient enrollment status by the pharmacy authorizes the pharmacy to provide to the healthcare setting or the patient a plurality of doses of the drug at a single treatment dose level according to an oral immunotherapy dosing schedule; (c) repeating step (b) at a different treatment dose level, wherein the pharmacy is not authorized to simultaneously provide the drug at more than one treatment dose level for the patient; (d) authorizing the provision of an oral immunotherapy treatment kit comprising a plurality of doses of a drug for treating a food allergy at different up-dosing levels to a healthcare setting, comprising: (i) receiving, at the first electronic device and from a third electronic device associated with a distributor enrolled in the drug distribution management database, a third healthcare setting enrollment verification request comprising healthcare setting information for a healthcare setting; (ii) generating, at the first electronic device, a third healthcare setting enrollment status, comprising (1) accessing the drug distribution management database, and (2) comparing the healthcare setting information in the third healthcare setting enrollment verification request to enrolled healthcare setting information in the drug distribution management database, and (3) obtaining a positive healthcare setting enrollment status by identifying a match between the healthcare setting information in the third healthcare setting enrollment verification request and the healthcare setting information in the drug distribution management database, or obtaining a negative healthcare setting enrollment status if no match between the healthcare setting information in the third healthcare setting enrollment verification request and the healthcare setting information in the drug distribution management database is identified; and (iii) sending, to the third electronic device, the third healthcare setting enrollment status, wherein receipt of a positive third healthcare setting enrollment status authorizes the distributor to provide the oral immunotherapy treatment kit to the healthcare setting.

Embodiment 53. A computer-implemented method for managing the distribution of a drug comprising a food allergen, comprising: (a) authorizing the provision of an initial dose escalation kit comprising plurality of different doses of the drug according to an initial dose escalation schedule, comprising: (i) receiving, at a first electronic device communicatively coupled to a drug distribution management database for the drug and from a second electronic device associated with a pharmacy enrolled in the drug distribution management database, a first healthcare setting enrollment verification request comprising healthcare setting information for a healthcare setting, a first healthcare provider enrollment verification request comprising healthcare provider information, and a first patient enrollment verification request comprising patient information for a patient; (ii) generating, at the first electronic device, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the healthcare setting enrollment verification request and the healthcare setting information in the drug distribution management database, or obtaining a negative healthcare setting enrollment status if no match between the healthcare setting information in the healthcare setting enrollment verification request and the healthcare setting information in the drug distribution management database is identified; (iii) generating, at the first electronic device, a first healthcare provider enrollment status, comprising (1) accessing the drug distribution management database, (2) comparing the healthcare provider information in the first healthcare provider enrollment verification request to enrolled healthcare provider information in the drug distribution management database, and (3) obtaining a positive healthcare provider enrollment status by identifying a match between the healthcare provider information in the healthcare provider enrollment verification request and the healthcare provider information in the drug distribution management database, or obtaining a negative healthcare provider enrollment status if no match between the healthcare provider information in the healthcare provider enrollment verification request and the healthcare provider information in the drug distribution management database is identified; (iv) generating, at the first electronic device, a first patient enrollment status, comprising (1) accessing the drug distribution management database, (2) comparing the patient information in the first patient enrollment verification request to enrolled patient information in the drug distribution management database, and (3) obtaining a positive patient enrollment status by identifying a match between the patient information in the patient enrollment verification request and the patient information in the drug distribution management database, or obtaining a negative patient enrollment status if no match between the patient information in the patient enrollment verification request and the patient information in the drug distribution management database is identified; and (v) sending, to the second electronic device, the first healthcare setting enrollment status, the first healthcare provider enrollment status, and the first patient enrollment status, wherein receipt of a positive first healthcare setting enrollment status, a positive healthcare provider enrollment status, and a positive first patient enrollment status by the pharmacy authorizes the pharmacy to provide to the healthcare setting or the healthcare provider an initial dose escalation kit associated with the patient, the initial dose escalation kit comprising a plurality of different doses of the drug according to an initial dose escalation schedule; (b) authorizing the provision of a plurality of doses of the drug at a single treatment dose level according to an oral immunotherapy dosing schedule, comprising: (i) receiving, at the first electronic device from the second electronic device, a second healthcare provider enrollment verification request comprising healthcare provider information for a healthcare provider and a second patient enrollment verification request comprising patient information for a patient; (ii) generating, at the first electronic device, a second healthcare provider enrollment status, comprising (1) accessing the drug distribution management database, (2) comparing the healthcare provider information in the second healthcare provider enrollment verification request to enrolled healthcare provider information in the drug distribution management database, and (3) obtaining a positive healthcare provider enrollment status by identifying a match between the healthcare provider information in the second healthcare provider enrollment verification request and the healthcare provider information in the drug distribution management database, or obtaining a negative healthcare provider enrollment status if no match between the healthcare provider information in the second healthcare provider enrollment verification request and the healthcare provider information in the drug distribution management database is identified; (iii) generating, at the first electronic device, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare provider enrollment status and the second patient enrollment status, wherein receipt of a positive second healthcare provider enrollment status and a positive second patient enrollment status by the pharmacy authorizes the pharmacy to provide to the healthcare setting, the healthcare provider, or the patient a plurality of doses of the drug at a single treatment dose level according to an oral immunotherapy dosing schedule; (c) repeating step (b) at a different treatment dose level, wherein the pharmacy is not authorized to simultaneously provide the drug at more than one treatment dose level for the patient; (d) authorizing the provision of an oral immunotherapy treatment kit comprising a plurality of doses of a drug for treating a food allergy at different up-dosing levels to a healthcare setting, comprising: (i) receiving, at the first electronic device and from a third electronic device associated with a distributor enrolled in the drug distribution management database, a second healthcare setting enrollment verification request comprising healthcare setting information for a healthcare setting; (ii) generating, at the first electronic device, a second healthcare setting enrollment status, comprising (1) accessing the drug distribution management database, and (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; and (iii) sending, to the third electronic device, the second healthcare setting enrollment status, wherein receipt of a positive third healthcare setting enrollment status authorizes the distributor to provide the oral immunotherapy treatment kit to the healthcare setting.

Embodiment 54. The method of embodiment 53, further comprising: receiving, by the first electronic device, a healthcare provider enrollment request comprising healthcare provider information for the patient; and adding, at the first electronic device, an entry comprising the healthcare provider information into the drug distribution management database to enroll the healthcare provider in the drug distribution management database.

Embodiment 55. The method of any one of embodiments 52-54, further comprising: receiving, by the first electronic device, a patient enrollment request comprising patient information for the patient; and adding, at the first electronic device, an entry comprising the patient information into the drug distribution management database to enroll the patient in the drug distribution management database.

Embodiment 56. The method of any one of embodiments 52-55, further comprising: receiving, by the first electronic device, a pharmacy enrollment request comprising pharmacy information for the pharmacy; and adding, at the first electronic device, an entry comprising the pharmacy information into the drug distribution management database to enroll the pharmacy in the drug distribution management database.

Embodiment 57. The method of any one of embodiments 52-56, further comprising: receiving, by the first electronic device, a healthcare setting enrollment request comprising healthcare setting information for the healthcare setting; and adding, at the first electronic device, an entry comprising the healthcare setting information into the drug distribution management database to enroll the healthcare setting in the drug distribution management database.

Embodiment 58. The method of any one of embodiments 52-57, further comprising: receiving, by the first electronic device, a distributor enrollment request comprising distributor information for the distributor; and adding, at the first electronic device, an entry comprising the distributor information into the drug distribution management database to enroll the distributor in the drug distribution management database.

Embodiment 59. A computer program product comprising instructions which, when the program is executed by a computer, causes the computer to carry out the steps of the method of any one of embodiments 52-58.

Embodiment 60. An electronic device comprising: one or more processors; a memory configured to store a drug distribution management database for a drug comprising a food allergen, the drug distribution management database comprising entries for enrolled patients comprising enrolled patient information, entries for enrolled healthcare settings comprising enrolled healthcare setting information, entries for enrolled pharmacies comprising enrolled pharmacy information, and entries for enrolled distributors comprising enrolled distributor information; and one or more programs stored on the memory configured to be executed by the one or more processors, the one or more programs including instructions for: (a) authorizing, by the one or more processors, the provision of an initial dose escalation kit comprising plurality of different doses of the drug according to an initial dose escalation schedule, comprising: (i) receiving, by the one or more processors and from a second electronic device associated with a pharmacy enrolled in the drug distribution management database, a first healthcare setting enrollment verification request comprising healthcare setting information for a healthcare setting and a first patient enrollment verification request comprising patient information for a patient; (ii) generating, by the one or more processors, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the first healthcare setting enrollment verification request and the healthcare setting information in the drug distribution management database, or obtaining a negative healthcare setting enrollment status if no match between the healthcare setting information in the first healthcare setting enrollment verification request and the healthcare setting information in the drug distribution management database is identified; (iii) generating, by the one or more processors, a first patient enrollment status, comprising (1) accessing the drug distribution management database, and (2) comparing the patient information in the first patient enrollment verification request to enrolled patient information in the drug distribution management database, and (3) obtaining a positive patient enrollment status by identifying a match between the patient information in the first patient enrollment verification request and the patient information in the drug distribution management database, or obtaining a negative patient enrollment status if no match between the patient information in the first patient enrollment verification request and the patient information in the drug distribution management database is identified; and (iv) sending, to the second electronic device, the first healthcare setting enrollment status and the first patient enrollment status, wherein receipt of a positive first healthcare setting enrollment status and a positive first patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule; (b) authorizing, by the one or more processors, a plurality of doses of the drug at a single treatment dose level according to an oral immunotherapy dosing schedule, comprising: (i) receiving, by the one or more processors and from the second electronic device, a second healthcare setting enrollment verification request comprising healthcare setting information for a healthcare setting and a second patient enrollment verification request comprising patient information for a patient; (ii) generating, by the one or more processors, a second healthcare setting enrollment status, comprising (1) accessing the drug distribution management database, (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; (iii) generating, by the one or more processors, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare setting enrollment status and the second patient enrollment status, wherein receipt of a positive second healthcare setting enrollment status and a positive second patient enrollment status by the pharmacy authorizes the pharmacy to provide to the healthcare setting or the patient a plurality of doses of the drug at a single treatment dose level according to an oral immunotherapy dosing schedule; (c) repeating step (b) at a different treatment dose level, wherein the pharmacy is not authorized to simultaneously provide the drug at more than one treatment dose level for the patient; (d) authorizing, by the one or more processors, the provision of an oral immunotherapy treatment kit comprising a plurality of doses of a drug for treating a food allergy at different up-dosing levels to a healthcare setting, comprising: (i) receiving, by the one or more processors and from a third electronic device associated with a distributor enrolled in the drug distribution management database, a third healthcare setting enrollment verification request comprising healthcare setting information for a healthcare setting; (ii) generating, by the one or more processors, a third healthcare setting enrollment status, comprising (1) accessing the drug distribution management database, (2) comparing the healthcare setting information in the third healthcare setting enrollment verification request to enrolled healthcare setting information in the drug distribution management database, and (3) obtaining a positive healthcare setting enrollment status by identifying a match between the healthcare setting information in the third healthcare setting enrollment verification request and the healthcare setting information in the drug distribution management database, or obtaining a negative healthcare setting enrollment status if no match between the healthcare setting information in the third healthcare setting enrollment verification request and the healthcare setting information in the drug distribution management database is identified; and (iii) sending, to the third electronic device, the third healthcare setting enrollment status, wherein receipt of a positive third healthcare setting enrollment status authorizes the distributor to provide the oral immunotherapy treatment kit to the healthcare setting.

Embodiment 61. An electronic device comprising: one or more processors; a memory configured to store a drug distribution management database for a drug comprising a food allergen, the drug distribution management database comprising entries for enrolled patients comprising enrolled patient information, entries for enrolled healthcare settings comprising enrolled healthcare setting information, entries for enrolled healthcare providers comprising enrolled healthcare provider information, entries for enrolled pharmacies comprising enrolled pharmacy information, and entries for enrolled distributors comprising enrolled distributor information; and one or more programs stored on the memory configured to be executed by the one or more processors, the one or more programs including instructions for: (a) authorizing, by the one or more processors, the provision of an initial dose escalation kit comprising plurality of different doses of the drug according to an initial dose escalation schedule, comprising: (i) receiving, by the one or more processors and from a second electronic device associated with a pharmacy enrolled in the drug distribution management database, a first healthcare setting enrollment verification request comprising healthcare setting information for a healthcare setting, a first healthcare provider enrollment verification request comprising healthcare provider information, and a first patient enrollment verification request comprising patient information for a patient; (ii) generating, by the one or more processors, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the first healthcare setting enrollment verification request and the healthcare setting information in the drug distribution management database, or obtaining a negative healthcare setting enrollment status if no match between the healthcare setting information in the first healthcare setting enrollment verification request and the healthcare setting information in the drug distribution management database is identified; (iii) generating, by the one or more processors, a first patient enrollment status, comprising (1) accessing the drug distribution management database, and (2) comparing the patient information in the first patient enrollment verification request to enrolled patient information in the drug distribution management database, and (3) obtaining a positive patient enrollment status by identifying a match between the patient information in the first patient enrollment verification request and the patient information in the drug distribution management database, or obtaining a negative patient enrollment status if no match between the patient information in the first patient enrollment verification request and the patient information in the drug distribution management database is identified; and (iv) generating, by the one or more processors, a first healthcare provider enrollment status, comprising (1) accessing the drug distribution management database, (2) comparing the healthcare provider information in the first healthcare provider enrollment verification request to enrolled healthcare provider information in the drug distribution management database, and (3) obtaining a positive healthcare provider enrollment status by identifying a match between the healthcare provider information in the first healthcare provider enrollment verification request and the healthcare provider information in the drug distribution management database, or obtaining a negative healthcare provider enrollment status if no match between the healthcare provider information in the first healthcare provider enrollment verification request and the healthcare provider information in the drug distribution management database is identified; (v) sending, to the second electronic device, the first healthcare setting enrollment status, the first healthcare provider enrollment status, and the first patient enrollment status, wherein receipt of a positive first healthcare setting enrollment status, a positive first healthcare provider enrollment status, and a positive first patient enrollment status by the pharmacy authorizes the pharmacy to provide to the healthcare setting or healthcare provider an initial dose escalation kit associated with the patient, the initial dose escalation kit comprising a plurality of different doses of the drug according to an initial dose escalation schedule; (b) authorizing, by the one or more processors, a plurality of doses of the drug at a single treatment dose level according to an oral immunotherapy dosing schedule, comprising: (i) receiving, by the one or more processors and from the second electronic device, a second healthcare provider enrollment verification request comprising healthcare provider information for a healthcare provider and a second patient enrollment verification request comprising patient information for a patient; (ii) generating, by the one or more processors, a second healthcare provider enrollment status, comprising (1) accessing the drug distribution management database, (2) comparing the healthcare provider information in the second healthcare provider enrollment verification request to enrolled healthcare provider information in the drug distribution management database, and (3) obtaining a positive healthcare provider enrollment status by identifying a match between the healthcare setting information in the second healthcare provider enrollment verification request and the healthcare provider information in the drug distribution management database, or obtaining a negative healthcare provider enrollment status if no match between the healthcare provider information in the second healthcare provider enrollment verification request and the healthcare provider information in the drug distribution management database is identified; (iii) generating, by the one or more processors, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare provider enrollment status and the second patient enrollment status, wherein receipt of a positive second healthcare provider enrollment status and a positive second patient enrollment status by the pharmacy authorizes the pharmacy to provide to the healthcare setting, the healthcare provider, or the patient a plurality of doses of the drug at a single treatment dose level according to an oral immunotherapy dosing schedule; (c) repeating step (b) at a different treatment dose level, wherein the pharmacy is not authorized to simultaneously provide the drug at more than one treatment dose level for the patient; (d) authorizing, by the one or more processors, the provision of an oral immunotherapy treatment kit comprising a plurality of doses of a drug for treating a food allergy at different up-dosing levels to a healthcare setting, comprising: (i) receiving, by the one or more processors and from a third electronic device associated with a distributor enrolled in the drug distribution management database, a second healthcare setting enrollment verification request comprising healthcare setting information for a healthcare setting; (ii) generating, by the one or more processors, a second healthcare setting enrollment status, comprising (1) accessing the drug distribution management database, (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; and (iii) sending, to the third electronic device, the second healthcare setting enrollment status, wherein receipt of a positive second healthcare setting enrollment status authorizes the distributor to provide the oral immunotherapy treatment kit to the healthcare setting.

Embodiment 62. The electronic device of embodiment 61, the one or more programs further including instructions for: receiving, by the one or more processors, a healthcare provider enrollment request comprising healthcare provider information for the healthcare provider; and adding, by the one or more processors, an entry comprising the healthcare provider information into the drug distribution management database to enroll the healthcare provider in the drug distribution management database.

Embodiment 63. The electronic device of any one of embodiments 60-62, the one or more programs further including instructions for: receiving, by the one or more processors, a pharmacy enrollment request comprising pharmacy information for the pharmacy; and adding, by the one or more processors, an entry comprising the pharmacy information into the drug distribution management database to enroll the pharmacy in the drug distribution management database.

Embodiment 64. The electronic device of any one of embodiments 60-63, the one or more programs further including instructions for: receiving, by the one or more processors, a distributor enrollment request comprising distributor information for the distributor; and adding, by the one or more processors, an entry comprising the distributor information into the drug distribution management database to enroll the distributor in the drug distribution management database.

Embodiment 65. The electronic device of any one of embodiments 60-64, the one or more programs further including instructions for: receiving, by the one or more processors, a patient enrollment request comprising patient information for the patient; and adding, by the one or more processors, an entry comprising the patient information into the drug distribution management database to enroll the patient in the drug distribution management database.

Embodiment 66. The electronic device of any one of embodiments 60-65, the one or more programs further including instructions for: receiving, by the one or more processors, a healthcare setting enrollment request comprising healthcare setting information for the healthcare setting; and adding, by the one or more processors, an entry comprising the healthcare setting information into the drug distribution management database to enroll the healthcare setting in the drug distribution management database.

Embodiment 67. A non-transitory computer-readable storage medium storing one or more programs comprising instructions, which when executed by one or more processors of an electronic device, cause the electronic device to: (a) authorize, by the one or more processors, the provision of an initial dose escalation kit comprising plurality of different doses of the drug comprising a food allergen according to an initial dose escalation schedule by: (i) receiving, by the one or more processors and from a second electronic device associated with a pharmacy enrolled in the drug distribution management database, a first healthcare setting enrollment verification request comprising healthcare setting information for a healthcare setting and a first patient enrollment verification request comprising patient information for a patient; (ii) generating, by the one or more processors, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the first healthcare setting enrollment verification request and the healthcare setting information in the drug distribution management database, or obtaining a negative healthcare setting enrollment status if no match between the healthcare setting information in the first healthcare setting enrollment verification request and the healthcare setting information in the drug distribution management database is identified; (iii) generating, by the one or more processors, a first patient enrollment status, comprising (1) accessing the drug distribution management database, and (2) comparing the patient information in the first patient enrollment verification request to enrolled patient information in the drug distribution management database, and (3) obtaining a positive patient enrollment status by identifying a match between the patient information in the first patient enrollment verification request and the patient information in the drug distribution management database, or obtaining a negative patient enrollment status if no match between the patient information in the first patient enrollment verification request and the patient information in the drug distribution management database is identified; and (iv) sending, to the second electronic device, the first healthcare setting enrollment status and the first patient enrollment status, wherein receipt of a positive first healthcare setting enrollment status and a positive first patient enrollment status by the pharmacy authorizes the pharmacy to provide to the healthcare setting an initial dose escalation kit associated with the patient, the initial dose escalation kit comprising a plurality of different doses of the drug according to an initial dose escalation schedule; (b) authorize, by the one or more processors, a plurality of doses of the drug at a single treatment dose level according to an oral immunotherapy dosing schedule by: (i) receiving, by the one or more processors and from the second electronic device, a second healthcare setting enrollment verification request comprising healthcare setting information for a healthcare setting and a second patient enrollment verification request comprising patient information for a patient; (ii) generating, by the one or more processors, a second healthcare setting enrollment status, comprising (1) accessing the drug distribution management database, (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; (iii) generating, by the one or more processors, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare setting enrollment status and the second patient enrollment status, wherein receipt of a positive second healthcare setting enrollment status and a positive second patient enrollment status by the pharmacy authorizes the pharmacy to provide to the healthcare setting or the patient a plurality of doses of the drug at a single treatment dose level according to an oral immunotherapy dosing schedule; (c) repeat step (b) at a different treatment dose level, wherein the pharmacy is not authorized to simultaneously provide the drug at more than one treatment dose level for the patient; (d) authorize, by the one or more processors, the provision of an oral immunotherapy treatment kit comprising a plurality of doses of a drug for treating a food allergy at different up-dosing levels to a healthcare setting, by: (i) receiving, by the one or more processors and from a third electronic device associated with a distributor enrolled in the drug distribution management database, a third healthcare setting enrollment verification request comprising healthcare setting information for a healthcare setting; (ii) generating, by the one or more processors, a third healthcare setting enrollment status, comprising (1) accessing the drug distribution management database, (2) comparing the healthcare setting information in the third healthcare setting enrollment verification request to enrolled healthcare setting information in the drug distribution management database, and (3) obtaining a positive healthcare setting enrollment status by identifying a match between the healthcare setting information in the third healthcare setting enrollment verification request and the healthcare setting information in the drug distribution management database, or obtaining a negative healthcare setting enrollment status if no match between the healthcare setting information in the third healthcare setting enrollment verification request and the healthcare setting information in the drug distribution management database is identified; and (iii) sending, to the third electronic device, the third healthcare setting enrollment status, wherein receipt of a positive third healthcare setting enrollment status authorizes the distributor to provide the oral immunotherapy treatment kit to the healthcare setting.

Embodiment 68. A non-transitory computer-readable storage medium storing one or more programs comprising instructions, which when executed by one or more processors of an electronic device, cause the electronic device to: (a) authorize, by the one or more processors, the provision of an initial dose escalation kit comprising plurality of different doses of the drug comprising a food allergen according to an initial dose escalation schedule by: (i) receiving, by the one or more processors and from a second electronic device associated with a pharmacy enrolled in the drug distribution management database, a first healthcare setting enrollment verification request comprising healthcare setting information for a healthcare setting, a first healthcare provider enrollment verification request comprising healthcare provider information, and a first patient enrollment verification request comprising patient information for a patient; (ii) generating, by the one or more processors, a first healthcare setting enrollment status, comprising (1) accessing the drug distribution management database, (2) comparing the healthcare setting information in the first healthcare setting enrollment verification request to enrolled healthcare setting information in the drug distribution management database, and (3) obtaining a positive healthcare setting enrollment status by identifying a match between the healthcare setting information in the first healthcare setting enrollment verification request and the healthcare setting information in the drug distribution management database, or obtaining a negative healthcare setting enrollment status if no match between the healthcare setting information in the first healthcare setting enrollment verification request and the healthcare setting information in the drug distribution management database is identified; (iii) generating, by the one or more processors, a first patient enrollment status, comprising (1) accessing the drug distribution management database, and (2) comparing the patient information in the first patient enrollment verification request to enrolled patient information in the drug distribution management database, and (3) obtaining a positive patient enrollment status by identifying a match between the patient information in the first patient enrollment verification request and the patient information in the drug distribution management database, or obtaining a negative patient enrollment status if no match between the patient information in the first patient enrollment verification request and the patient information in the drug distribution management database is identified; (iv) generating, by the one or more processors, a first healthcare provider enrollment status, comprising (1) accessing the drug distribution management database, (2) comparing the healthcare provider information in the first healthcare provider enrollment verification request to enrolled healthcare provider information in the drug distribution management database, and (3) obtaining a positive healthcare provider enrollment status by identifying a match between the healthcare provider information in the first healthcare provider enrollment verification request and the healthcare provider information in the drug distribution management database, or obtaining a negative healthcare provider enrollment status if no match between the healthcare provider information in the first healthcare provider enrollment verification request and the healthcare setting information in the drug distribution management database is identified; (v) sending, to the second electronic device, the first healthcare setting enrollment status, the first healthcare provider enrollment status, and the first patient enrollment status, wherein receipt of a positive first healthcare setting enrollment status, a positive first healthcare provider enrollment status, and a positive first patient enrollment status by the pharmacy authorizes the pharmacy to provide to the healthcare setting or healthcare provider an initial dose escalation kit associated with the patient, the initial dose escalation kit comprising a plurality of different doses of the drug according to an initial dose escalation schedule; (b) authorize, by the one or more processors, a plurality of doses of the drug at a single treatment dose level according to an oral immunotherapy dosing schedule by: (i) receiving, by the one or more processors and from the second electronic device, a second healthcare provider enrollment verification request comprising healthcare provider information for a healthcare provider and a second patient enrollment verification request comprising patient information for a patient; (ii) generating, by the one or more processors, a second healthcare provider enrollment status, comprising (1) accessing the drug distribution management database, (2) comparing the healthcare provider information in the second healthcare provider enrollment verification request to enrolled healthcare provider information in the drug distribution management database, and (3) obtaining a positive healthcare provider enrollment status by identifying a match between the healthcare provider information in the second healthcare provider enrollment verification request and the healthcare provider information in the drug distribution management database, or obtaining a negative healthcare provider enrollment status if no match between the healthcare provider information in the second healthcare provider enrollment verification request and the healthcare provider information in the drug distribution management database is identified; (iii) generating, by the one or more processors, a second patient enrollment status, comprising (1) accessing the drug distribution management database, (2) comparing the patient information in the second patient enrollment verification request to enrolled patient information in the drug distribution management database, and (3) obtaining a positive patient enrollment status by identifying a match between the patient information in the second patient enrollment verification request and the patient information in the drug distribution management database, or obtaining a negative patient enrollment status if no match between the patient information in the second patient enrollment verification request and the patient information in the drug distribution management database is identified; and (iv) sending, to the second electronic device, the second healthcare provider enrollment status and the second patient enrollment status, wherein receipt of a positive second healthcare provider enrollment status and a positive second patient enrollment status by the pharmacy authorizes the pharmacy to provide to the healthcare setting, the healthcare provider, or the patient a plurality of doses of the drug at a single treatment dose level according to an oral immunotherapy dosing schedule; (c) repeat step (b) at a different treatment dose level, wherein the pharmacy is not authorized to simultaneously provide the drug at more than one treatment dose level for the patient; (d) authorize, by the one or more processors, the provision of an oral immunotherapy treatment kit comprising a plurality of doses of a drug for treating a food allergy at different up-dosing levels to a healthcare setting, by: (i) receiving, by the one or more processors and from a third electronic device associated with a distributor enrolled in the drug distribution management database, a second healthcare setting enrollment verification request comprising healthcare setting information for a healthcare setting; (ii) generating, by the one or more processors, a second healthcare setting enrollment status, comprising (1) accessing the drug distribution management database, (2) comparing the healthcare setting information in the second healthcare setting enrollment verification request to enrolled healthcare setting information in the drug distribution management database, and (3) obtaining a positive healthcare setting enrollment status by identifying a match between the healthcare setting information in the second healthcare setting enrollment verification request and the healthcare setting information in the drug distribution management database, or obtaining a negative healthcare setting enrollment status if no match between the healthcare setting information in the second healthcare setting enrollment verification request and the healthcare setting information in the drug distribution management database is identified; and (iii) sending, to the third electronic device, the second healthcare setting enrollment status, wherein receipt of a positive third healthcare setting enrollment status authorizes the distributor to provide the oral immunotherapy treatment kit to the healthcare setting.

Embodiment 69. The storage medium of embodiment 68, wherein the instructions, which when executed by the one or more processors of the electronic device, further cause the electronic device to: receive, by the one or more processors, a healthcare provider enrollment request comprising healthcare provider information for the healthcare provider; and add, by the one or more processors, an entry comprising the healthcare provider information into the drug distribution management database to enroll the healthcare provider in the drug distribution management database.

Embodiment 70. The storage medium of any one of embodiments 67-69, wherein the instructions, which when executed by the one or more processors of the electronic device, further cause the electronic device to: receive, by the one or more processors, a pharmacy enrollment request comprising pharmacy information for the pharmacy; and add, by the one or more processors, an entry comprising the pharmacy information into the drug distribution management database to enroll the pharmacy in the drug distribution management database.

Embodiment 71. The storage medium of any one of embodiments 67-70, wherein the instructions, which when executed by the one or more processors of the electronic device, further cause the electronic device to: receive, by the one or more processors, a distributor enrollment request comprising distributor information for the distributor; and add, by the one or more processors, an entry comprising the distributor information into the drug distribution management database to enroll the distributor in the drug distribution management database.

Embodiment 72. The storage medium of any one of embodiments 67-71, wherein the instructions, which when executed by the one or more processors of the electronic device, further cause the electronic device to: receive, by the one or more processors, a patient enrollment request comprising patient information for the patient; and add, by the one or more processors, an entry comprising the patient information into the drug distribution management database to enroll the patient in the drug distribution management database.

Embodiment 73. The storage medium of any one of embodiments 67-72, wherein the instructions, which when executed by the one or more processors of the electronic device, further cause the electronic device to: receive, by the one or more processors, a healthcare setting enrollment request comprising healthcare setting information for the healthcare setting; and add, by the one or more processors, an entry comprising the healthcare setting information into the drug distribution management database to enroll the healthcare setting in the drug distribution management database.

Embodiment 74. A method of treating a patient for a food allergy by oral immunotherapy, comprising: (a) enrolling the patient or verifying enrollment of the patient in a drug distribution management database for a drug comprising a food allergen; (b) administering to the enrolled patient, in a healthcare setting enrolled in the drug distribution management database, a dose of the drug at a first up-dosing level according to an up-dosing schedule; (c) monitoring the enrolled patient, in the enrolled healthcare setting, for an allergic response to the drug after administering the dose of the drug in step (b); (d) providing a first prescription requesting a pharmacy enrolled in the drug distribution management database release to the enrolled patient or the enrolled healthcare setting a plurality of doses of the drug at the first up-dosing level; (e) administering to the enrolled patient, in the enrolled healthcare setting, a dose of the drug at a subsequent up-dosing level according to the up-dosing schedule; (f) monitoring the enrolled patient, in the enrolled healthcare setting, for an allergic response to the drug after administering the dose of the drug in step (e); and (g) providing a subsequent prescription requesting the enrolled pharmacy release to the enrolled patient or the enrolled healthcare setting a plurality of doses of the drug at the subsequent up-dosing level.

Embodiment 75. The method of embodiment 74, wherein the dose of (b) or (e) is administered under the supervision of a healthcare provider enrolled in the drug distribution management database.

Embodiment 76. The method of embodiment 74 or embodiment 75, wherein the monitoring of (c) or (f) is under the supervision of a healthcare provider enrolled in the drug distribution management database.

Embodiment 77. The method of any one of embodiments 74-76 comprising obtaining the dose of the drug administered to the enrolled patient in step (b) or step (e) from an oral immunotherapy treatment kit comprising a plurality of doses of the drug at different up-dosing levels.

Embodiment 78. The method of embodiment 77, wherein the oral immunotherapy treatment kit is received from a distributor enrolled in the drug distribution management database.

Embodiment 79. The method of embodiment 78, comprising submitting a request to the enrolled distributor to provide the oral immunotherapy treatment kit to the enrolled healthcare setting, wherein providing the oral immunotherapy treatment kit to the enrolled healthcare setting is conditioned on the enrolled distributor verifying enrollment of the healthcare setting.

Embodiment 80. The method of any one of embodiments 74-79, comprising, prior to step (b), administering to the enrolled patient, in the enrolled healthcare setting, a plurality of different doses of the drug according to an initial dose escalation schedule.

Embodiment 81. The method of embodiment 80, wherein the plurality of different doses of the drug according to the initial dose escalation schedule are administered under the supervision of a healthcare provider enrolled in the drug distribution management database.

Embodiment 82. The method of embodiment 60 or embodiment 61, further comprising monitoring the enrolled patient, in the enrolled healthcare setting, for an allergic response to the drug after administering each different dose of the drug according to the initial dose escalation schedule.

Embodiment 83. The method of embodiment 82, wherein the monitoring after administering each different dose of the drug according to the initial dose escalation schedule is under the supervision of a healthcare provider enrolled in the drug distribution management database.

Embodiment 84. The method of any one of embodiments 80-83, wherein the plurality of different doses of the drug according to the initial dose escalation schedule is obtained from an initial dose escalation kit assigned to the enrolled patient.

Embodiment 85. The method of embodiment 84, comprising receiving the initial dose escalation kit from the enrolled pharmacy.

Embodiment 86. The method of embodiment 85, comprising submitting a request to the enrolled pharmacy to provide the initial dose escalation kit to the enrolled healthcare setting, wherein providing the initial dose escalation kit to the enrolled healthcare setting is conditioned on the enrolled pharmacy verifying (1) enrollment of the patient in the drug distribution management database and (2) enrollment of the healthcare setting.

Embodiment 87. The method of embodiment 86, wherein providing the initial dose escalation kit to the enrolled healthcare setting is further conditioned on the enrolled pharmacy verifying enrollment of a healthcare provider in the drug distribution management database, wherein the healthcare provider is associated with the enrolled patient.

Embodiment 88. A method of initiating treatment of a patient for a food allergy by oral immunotherapy, comprising: (a) enrolling the patient or verifying enrollment of the patient in a drug distribution management database for a drug comprising a food allergen; (b) administering to the enrolled patient, in the enrolled healthcare setting, a plurality of different doses of the drug according to an initial dose escalation schedule; and (c) monitoring the enrolled patient, in the enrolled healthcare setting, for an allergic response to the drug after administering each different dose of the drug according to the initial dose escalation schedule in step (b).

Embodiment 89. The method of embodiment 88, wherein the plurality of different doses of step (b) are administered under the supervision of a healthcare provider enrolled in the drug distribution management database.

Embodiment 90. The method of embodiment 88 or embodiment 89, wherein the monitoring of step (c) is under the supervision of a healthcare provider enrolled in the drug distribution management database.

Embodiment 91. The method of any one of embodiments 88-90, wherein the plurality of different doses of the drug according to the initial dose escalation schedule are obtained from an initial dose escalation kit assigned to the enrolled patient.

Embodiment 92. The method of embodiment 91, comprising receiving the initial dose escalation kit from a pharmacy enrolled in the drug distribution management database.

Embodiment 93. The method of embodiment 92, comprising submitting a request to the enrolled pharmacy to provide the initial dose escalation kit to the enrolled healthcare setting, wherein providing the initial dose escalation kit to the enrolled healthcare setting is conditioned on the enrolled pharmacy verifying (1) enrollment of the patient in the drug distribution management database and (2) enrollment of the healthcare setting.

Embodiment 94. The method of embodiment 93, wherein providing the initial dose escalation kit to the enrolled healthcare setting is further conditioned on the enrolled pharmacy verifying enrollment of a healthcare provider in the drug distribution management database, wherein the healthcare provider is associated with the enrolled patient.

Embodiment 95. A method of continuing treatment of a patient for a food allergy by oral immunotherapy, comprising: (a) enrolling the patient or verifying enrollment of the patient in a drug distribution management database for a drug comprising a food allergen; (b) administering to the patient, in the enrolled healthcare setting and after enrolling the patient or verifying enrollment of the patient in step (a), a dose of the drug at a dose level higher than any than any previous dose level administered to the patient; and (c) monitoring the patient, in the enrolled healthcare setting, for an allergic response to the drug after administering the drug in step (b).

Embodiment 96. The method of embodiment 95, wherein the dose of step (b) is administered under supervision of a healthcare provider enrolled in the drug distribution management database.

Embodiment 97. The method of embodiment 95 or embodiment 96, wherein the monitoring of step (c) is under the supervision of a healthcare provider enrolled in the drug distribution management database.

Embodiment 98. The method of any one of embodiments 95-97, wherein the dose of the drug is obtained from an oral immunotherapy dosing kit comprising a plurality of doses of the drug at different dose levels.

Embodiment 99. The method of embodiment 98, wherein doses of the drug obtained from the oral immunotherapy dosing kit are administered only to patients enrolled in the drug distribution management database.

Embodiment 100. The method of embodiment 98 or 99, wherein doses of the drug obtained from the oral immunotherapy dosing kit are not used to initiate treatment of the patient.

Embodiment 101. The method of any one of embodiments 98-100, wherein the oral immunotherapy treatment kit is received from a distributor enrolled in the drug distribution management database.

Embodiment 102. The method of embodiment 101, comprising submitting a request to the enrolled distributor to provide the oral immunotherapy treatment kit to the enrolled healthcare setting, wherein providing the oral immunotherapy treatment kit to the enrolled healthcare setting is conditioned on the enrolled distributor verifying enrollment of the healthcare setting.

Embodiment 103. The method of any one of embodiments 74-102, comprising submitting an enrollment request for the healthcare setting, and receiving an enrollment verification for the healthcare setting.

Embodiment 104. The method of embodiment 103, wherein the enrollment request affirms that the healthcare setting is equipped to identify and treat a systemic allergic reaction.

Embodiment 105. The method of embodiment 103 or 104, wherein the enrollment request affirms that the healthcare setting will monitor the patient for an allergic response to the drug after administering the drug to the patient.

Embodiment 106. The method of any one of embodiments 103-105, wherein the enrollment request affirms that the healthcare setting will, for every patient to be treated with the drug by the healthcare setting, enroll the patient in or verify enrollment of the patient in the drug distribution management database.

Embodiment 107. The method of any one of embodiments 103-106, wherein the enrollment request affirms that the healthcare setting will only use an initial dose escalation kit for initiating treatment of a patient uniquely associated with the initial dose escalation kit.

Embodiment 108. The method of any one of embodiment 74-107, comprising generating a record for each dose of the drug administered to the enrolled patient in the enrolled healthcare setting, wherein the record identifies the administration of the drug and the monitoring of the enrolled patient following administration of the drug.

Embodiment 109. The method of any one of embodiments 74-108, further comprising recommending to the patient that the patient have an anaphylaxis-response treatment immediately available to the patient during the course of treatment.

Embodiment 110. The method of embodiment 109, wherein the anaphylaxis response treatment comprises epinephrine.

Embodiment 111. The method of embodiment 109 or 110, comprising generating a record confirming that the recommendation that the patient have an anaphylaxis-response treatment immediately available to the patient during the course of treatment has been made to the patient.

Embodiment 112. The method of any one of embodiments 74-111, further comprising recommending to the patient that the patient avoid dietary consumption of the food during the course of treatment.

Embodiment 113. The method of embodiment 112, comprising generating a record confirming that the recommendation that the patient avoid dietary consumption of the food during the course of treatment has been made to the patient.

Embodiment 114. The method of any one of embodiments 74-113, comprising providing a patient status to a drug distribution management database manager upon discontinuation of treatment by the patient or transfer of care of the patient to a subsequent healthcare setting.

Embodiment 115. A method of providing a drug comprising a food allergen to a healthcare setting or a patient, comprising: (a) receiving, at a pharmacy enrolled in a drug distribution management database for the drug, a request to provide to a healthcare setting an initial dose escalation kit associated with an identified patient, the initial dose escalation kit comprising a plurality of different doses of the drug according to an initial dose escalation schedule; (b) verifying enrollment of the identified patient in the drug distribution management database; (c) verifying enrollment of the healthcare setting in the drug distribution management database; (d) providing, to the healthcare setting, the initial dose escalation kit associated with the identified patient; (e) receiving, at the enrolled pharmacy, a prescription issued by the healthcare setting to provide a plurality of doses of the drug at an up-dosing level according to an up-dosing schedule or a plurality of doses of the drug at a maintenance level; (f) repeating steps (b) and (c) after step (e); and (g) providing, to the patient or the healthcare setting, the plurality of doses of the drug at the up-dosing level or the plurality of doses of the drug at the maintenance level.

Embodiment 116. A method of providing a drug comprising a food allergen to a healthcare setting, comprising: (a) receiving, at a pharmacy enrolled in a drug distribution management database for the drug, a request to provide to a healthcare setting an initial dose escalation kit associated with an identified patient, the initial dose escalation kit comprising a plurality of different doses of the drug according to an initial dose escalation schedule; (b) verifying enrollment of the identified patient in a drug distribution management database for the drug; (c) verifying enrollment of the healthcare setting in the drug distribution management database; and (d) providing, to the healthcare setting, the initial dose escalation kit associated with the identified patient.

Embodiment 117. A method of providing a drug comprising a food allergen to a patient or healthcare setting, comprising: (a) receiving, at a pharmacy enrolled in a drug distribution management database for the drug, a prescription issued by the healthcare setting to provide a plurality of doses of the drug at an up-dosing level according to an up-dosing schedule or a plurality of doses of the drug at a maintenance level; (b) verifying enrollment of the identified patient in a drug distribution management database for the drug; (c) verifying enrollment of the healthcare setting in the drug distribution management database; and (d) providing, to the patient or the healthcare setting, the plurality of doses of the drug at the up-dosing level or the plurality of doses of the drug at the maintenance level.

Embodiment 118. The method of any one of embodiments 115-117, comprising submitting an enrollment request for the pharmacy, and receiving an enrollment verification for the pharmacy.

Embodiment 119. The method of any one of embodiments 115-118, comprising generating a record for the patient that associates a treatment level of the provided plurality of doses with the identified patient, wherein the treatment level indicates an initial dose escalation level, the up-dosing level, or a maintenance dose level.

Embodiment 120. A method of providing an oral immunotherapy treatment kit comprising a plurality of doses of a drug comprising a food allergen at different levels, comprising: (a) receiving, at a distributor enrolled in a drug distribution management database for the drug, a request to provide the oral immunotherapy treatment kit to a healthcare setting; (b) verifying enrollment of the healthcare setting in the drug distribution management database for the drug; and (c) providing the oral immunotherapy treatment kit to the healthcare setting only if the healthcare setting is enrolled in the drug distribution management database.

Embodiment 121. The method of embodiment 120, comprising submitting an enrollment request for the distributor, and receiving an enrollment verification for the distributor.

Embodiment 122. The method of embodiment 120 or 121, comprising generating a record identifying the healthcare setting that has been provided the oral immunotherapy treatment kit.

Embodiment 123. The method of any one of embodiments 120-122, wherein the request to provide the oral immunotherapy treatment kit to the healthcare setting is made by the healthcare setting.

Embodiment 124. A method of treating a patient for a food allergy by oral immunotherapy, comprising: (a) enrolling the patient or verifying enrollment of the patient in a drug distribution management database for a drug comprising a food allergen; (b) administering to the enrolled patient a dose of the drug at a first up-dosing level according to an up-dosing schedule, wherein the dose is administered under the supervision of a healthcare provider enrolled in the drug distribution management database; (c) monitoring the enrolled patient, under the supervision of the enrolled healthcare provider, for an allergic response to the drug after administering the dose of the drug in step (b); (d) providing a first prescription requesting a pharmacy enrolled in the drug distribution management database release to the enrolled patient or the enrolled healthcare provider a plurality of doses of the drug at the first up-dosing level; (e) administering to the enrolled patient a dose of the drug at a subsequent up-dosing level according to the up-dosing schedule, wherein the dose is administered under the supervision of the healthcare provider; (f) monitoring the enrolled patient for an allergic response to the drug after administering the dose of the drug in step (e); and (g) providing a subsequent prescription requesting the enrolled pharmacy release to the enrolled patient, the enrolled healthcare provider, or a healthcare setting enrolled in the drug distribution management database a plurality of doses of the drug at the subsequent up-dosing level.

Embodiment 125. The method of embodiment 124, further comprising counseling the enrolled patient, wherein counseling is performed under the supervision of the enrolled healthcare provider.

Embodiment 126. The method of embodiment 124 or embodiment 125, wherein the drug administered to the enrolled patient in step (b) or step (e) is administered in a healthcare setting enrolled in the drug distribution management database.

Embodiment 127. The method of any one of embodiments 124-126, comprising obtaining the dose of the drug administered to the enrolled patient in step (b) or step (e) from an oral immunotherapy treatment kit comprising a plurality of doses of the drug at different up-dosing levels.

Embodiment 128. The method of embodiment 127, wherein the oral immunotherapy treatment kit is received from a distributor enrolled in the drug distribution management database.

Embodiment 129. The method of embodiment 128, comprising submitting a request to the enrolled distributor to provide the oral immunotherapy treatment kit to the enrolled healthcare provider or enrolled healthcare setting, wherein providing the oral immunotherapy treatment kit to the enrolled healthcare provider or enrolled healthcare setting is conditioned on the enrolled distributor verifying enrollment of the healthcare provider if the oral immunotherapy treatment kit is to be provided to the healthcare provider, or verifying enrollment of the healthcare setting if the oral immunotherapy treatment kit is to be provided to the healthcare setting.

Embodiment 130. The method of any one of embodiments 124-129, comprising, prior to step (b), administering to the enrolled patient a plurality of different doses of the drug according to an initial dose escalation schedule, wherein the administration is under the supervision of the enrolled healthcare provider.

Embodiment 131. The method of embodiment 130, further comprising monitoring the enrolled patient for an allergic response to the drug after administering each different dose of the drug according to the initial dose escalation schedule.

Embodiment 132. The method of embodiment 130 or embodiment 131, wherein the plurality of different doses of the drug according to the initial dose escalation schedule is obtained from an initial dose escalation kit assigned to the enrolled patient.

Embodiment 133. The method of embodiment 132, comprising receiving the initial dose escalation kit from the enrolled pharmacy.

Embodiment 134. The method of embodiment 133, comprising submitting a request to the enrolled pharmacy to provide the initial dose escalation kit to the enrolled healthcare provider, wherein providing the initial dose escalation kit to the enrolled healthcare provider is conditioned on the enrolled pharmacy verifying (1) enrollment of the patient in the drug distribution management database and (2) enrollment of the healthcare provider.

Embodiment 135. The method of embodiment 134, wherein providing the initial dose escalation kit to the enrolled healthcare provider is further conditioned on the enrolled pharmacy verifying enrollment of a healthcare setting in the drug distribution management database, wherein the initial dose escalation kit is to be administered to the enrolled patient in the enrolled healthcare setting.

Embodiment 136. A method of initiating treatment of a patient for a food allergy by oral immunotherapy, comprising: (a) enrolling the patient or verifying enrollment of the patient in a drug distribution management database for a drug comprising a food allergen; (b) administering to the enrolled patient a plurality of different doses of the drug according to an initial dose escalation schedule, wherein the administration is under the supervision of a healthcare provider enrolled in the drug distribution management database; and (c) monitoring the enrolled patient for an allergic response to the drug after administering each different dose of the drug according to the initial dose escalation schedule in step (b).

Embodiment 137. The method of embodiment 136, wherein the administrations of step (b) are in a healthcare setting enrolled in the drug distribution management database.

Embodiment 138. The method of embodiment 136 or embodiment 137, wherein the monitoring of step (c) is in a healthcare setting enrolled in the drug distribution management database.

Embodiment 139. The method of any one of embodiments 136-138, wherein the plurality of different doses of the drug according to the initial dose escalation schedule are obtained from an initial dose escalation kit assigned to the enrolled patient.

Embodiment 140. The method of embodiment 139, comprising receiving the initial dose escalation kit from a pharmacy enrolled in the drug distribution management database.

Embodiment 141. The method of embodiment 140, comprising submitting a request to the enrolled pharmacy to provide the initial dose escalation kit to the enrolled healthcare provider or a healthcare setting enrolled in the drug distribution management database, wherein providing the initial dose escalation kit is conditioned on verifying enrollment of the patient in the drug distribution management database and: i) verifying enrollment of the healthcare provider in the drug distribution management database if the initial dose escalation kit is to be provided to the healthcare provider, or ii) verifying enrollment of the healthcare setting in the drug distribution management database if the initial dose escalation kit is to be provided to the healthcare setting.

Embodiment 142. A method for authorizing the provision of a drug comprising a food allergen to a healthcare setting, comprising: (a) verifying enrollment of the healthcare setting in a drug distribution management database; (b) verifying enrollment of a patient in the drug distribution management database; (c) authorizing, after enrollment of the healthcare setting and the patient in the drug distribution management database has been verified, a pharmacy to provide to the healthcare setting an initial dose escalation kit associated with the patient, the initial dose escalation kit comprising a plurality of different doses of the drug according to an initial dose escalation schedule.

Embodiment 143. The method of embodiment 142, further comprising verifying enrollment of a healthcare provider in the drug distribution management database, wherein step (c) occurs after enrollment of the healthcare provider in the drug distribution management database has been verified.

Embodiment 144. A method for authorizing the provision of a drug comprising a food allergen to a healthcare setting, comprising: (a) receiving a healthcare setting enrollment verification request and a patient enrollment verification request; (b) generating a healthcare setting enrollment status indicating whether the healthcare setting associated with the healthcare setting enrollment verification request is enrolled in a drug distribution management database; (c) generating a patient enrollment status indicating whether a patient associated with the patient enrollment verification request is enrolled in the drug distribution management database; and (d) sending the healthcare setting enrollment status and the patient enrollment status to a pharmacy enrolled in the drug distribution management database to authorize the pharmacy to provide to the healthcare setting an initial dose escalation kit associated with the patient, the initial dose escalation kit comprising a plurality of different doses of the drug according to an initial dose escalation schedule.

Embodiment 145. The method of embodiment 144, further comprising: receiving a healthcare provider enrollment verification request; generating a healthcare provider enrollment status indicating whether a healthcare provider associated with the healthcare provider enrollment verification request is enrolled in the drug distribution management database; and sending the healthcare provider enrollment status to the pharmacy enrolled in the drug distribution management database to authorize the pharmacy to provide to the healthcare setting an initial dose escalation kit associated with the patient.

Embodiment 146. A method of authorizing the provision of a drug comprising a food allergen to a healthcare setting or a patient, comprising: (a) verifying enrollment of a healthcare provider in a drug distribution management database; (b) verifying enrollment of a patient in the drug distribution management database; (c) authorizing, after enrollment of the healthcare provider and the patient in the drug distribution management database has been verified, a pharmacy to provide to the healthcare setting or the patient a plurality of doses of the drug at a single treatment dose level according to an oral immunotherapy dosing schedule.

Embodiment 147. The method of embodiment 146, further comprising verifying enrollment of a healthcare setting in a drug distribution management database, wherein step (c) occurs after enrollment of the healthcare setting in the drug distribution management database has been verified.

Embodiment 148. A method for authorizing the provision of a drug comprising a food allergen to a healthcare setting or a patient, comprising: (a) receiving a healthcare provider enrollment verification request and a patient enrollment verification request; (b) generating a healthcare provider enrollment status indicating whether a healthcare provider associated with the healthcare setting enrollment verification request is enrolled in a drug distribution management database; (c) generating a patient enrollment status indicating whether the patient associated with the patient enrollment verification request is enrolled in the drug distribution management database; and (d) sending the healthcare provider enrollment status and the patient enrollment status to a pharmacy enrolled in the drug distribution management database to authorize the pharmacy to provide to the healthcare setting or the patient a plurality of doses of the drug at a single treatment dose level according to an oral immunotherapy dosing schedule.

Embodiment 149. The method of embodiment 148, further comprising repeating steps (a)-(d) at a plurality of different treatment dose levels, wherein the pharmacy is not authorized to simultaneously provide the drug at more than one treatment dose level for the patient. 150. A method for authorizing the provision of a drug comprising a food allergen to a pharmacy, comprising: (a) verifying enrollment of the pharmacy in a drug distribution management database; (b) authorizing, after enrollment of the pharmacy in the drug distribution management database has been verified, a distributor to provide the drug to the pharmacy.

Embodiment 150. A method for authorizing the provision of a drug comprising a food allergen to a pharmacy, comprising: (a) receiving a pharmacy enrollment verification request; (b) generating a pharmacy enrollment status indicating whether the pharmacy associated with the pharmacy enrollment verification request is enrolled in a drug distribution management database; (c) sending the pharmacy enrollment status to a distributor enrolled in the drug distribution management database to authorize the distributor to provide the drug to the pharmacy.

Embodiment 151. A method for authorizing the distribution of an oral immunotherapy treatment kit comprising a plurality of doses of a drug comprising a food allergen at different dosing levels to a healthcare setting, comprising: (a) verifying enrollment of the healthcare setting in a drug distribution management database; (b) authorizing, after enrollment of the healthcare setting in the drug distribution management database has been verified, a distributor to provide the oral immunotherapy treatment kit to the healthcare setting.

Embodiment 152. A method for authorizing the distribution of an oral immunotherapy treatment kit comprising a plurality of doses of a drug comprising a food allergen at different dosing levels to a healthcare setting, comprising: (a) receiving a healthcare setting enrollment verification request; (b) generating a healthcare setting enrollment status indicating whether the healthcare setting associated with the pharmacy enrollment verification request is enrolled in a drug distribution management database; (c) sending the healthcare setting enrollment status to a distributor enrolled in the drug distribution management database to authorize the distributor to provide the oral immunotherapy treatment kit to the healthcare setting.

Embodiment 153. A method for managing the distribution of a drug comprising a food allergen, comprising: (a) authorizing the provision of an initial dose escalation kit comprising plurality of different doses of the drug according to an initial dose escalation schedule, comprising: (i) verifying enrollment of a healthcare setting in a drug distribution management database; (ii) verifying enrollment of a patient in a drug distribution management database; (iii) authorizing, after enrollment of the healthcare setting and the patient in the drug distribution management database has been verified, a pharmacy to provide to the healthcare setting the initial dose escalation kit associated with the patient; (b) authorizing the provision of a plurality of doses of the drug at a single treatment dose level according to an oral immunotherapy dosing schedule, comprising: (i) verifying enrollment of a healthcare provider in the drug distribution management database; (ii) verifying enrollment of a patient in the drug distribution management database; (iii) authorizing, after enrollment of the healthcare provider and the patient in the drug distribution management database has been verified, the pharmacy to provide to the healthcare setting or the patient the plurality of doses of the drug at the single treatment dose level; (c) repeating step (b) at a different treatment dose level, wherein the pharmacy is not authorized to simultaneously provide the drug at more than one treatment dose level for the patient; and (d) authorizing the provision of an oral immunotherapy treatment kit comprising a plurality of doses of a drug for treating a food allergy at different up-dosing levels to the healthcare setting, comprising: (i) verifying enrollment of the healthcare setting in the drug distribution management database; (ii) authorizing, after enrollment of the healthcare setting in the drug distribution management database has been verified, a distributor to provide the oral immunotherapy treatment kit to the healthcare setting.

Embodiment 154. The method of embodiment 153, wherein step (a) further comprising verifying enrollment of a healthcare provider in the drug distribution management database, wherein step (a)(iii) occurs after enrollment of the healthcare provider in the drug distribution management database has been verified.

Embodiment 155. A method for managing the distribution of a drug comprising a food allergen, comprising: (a) authorizing the provision of an initial dose escalation kit comprising plurality of different doses of the drug according to an initial dose escalation schedule, comprising: (i) receiving a healthcare setting enrollment verification request and a patient enrollment verification request; (ii) generating a healthcare setting enrollment status indicating whether a healthcare setting associated with the healthcare setting enrollment verification request is enrolled in the drug distribution management database; (iii) generating a patient enrollment status indicating whether a patient associated with the patient enrollment verification request is enrolled in the drug distribution management database; and (iv) sending the healthcare setting enrollment status and the patient enrollment status to a pharmacy enrolled in the drug distribution management database to authorize the pharmacy to provide to the healthcare setting the initial dose escalation kit associated with the patient; (b) authorizing the provision of a plurality of doses of the drug at a single treatment dose level according to an oral immunotherapy dosing schedule, comprising: (i) verifying enrollment of the healthcare provider in the drug distribution management database; (ii) verifying enrollment of the patient in the drug distribution management database; (iii) authorizing, after enrollment of the healthcare provider and the patient in the drug distribution management database has been verified, the pharmacy to provide to the healthcare setting or the patient a plurality of doses of the drug at a single treatment dose level; (c) repeating step (b) at a different treatment dose level, wherein the pharmacy is not authorized to simultaneously provide the drug at more than one treatment dose level for the patient; and (d) authorizing the provision of an oral immunotherapy treatment kit comprising a plurality of doses of a drug for treating a food allergy at different up-dosing levels to the healthcare setting, comprising: (i) receiving a healthcare setting enrollment verification request; (ii) generating a healthcare setting enrollment status indicating whether a healthcare setting associated with the pharmacy enrollment verification request is enrolled in a drug distribution management database; (iii) sending the healthcare setting enrollment status to a distributor enrolled in the drug distribution management database to authorize the distributor to provide the oral immunotherapy treatment kit to the healthcare setting.

Embodiment 156. The method of embodiment 155, wherein step (a) further comprises: receiving a healthcare provider enrollment verification request; generating a healthcare provider enrollment status indicating whether a healthcare provider associated with the healthcare provider enrollment verification request is enrolled in a drug distribution management database; and sending the healthcare provider enrollment status to the pharmacy enrolled in the drug distribution management database to authorize the pharmacy to provide to the healthcare setting an initial dose escalation kit associated with the patient.

Embodiment 157. The method of any one of embodiments 142-156, further comprising: receiving a pharmacy enrollment request comprising patient information for the patient; and enrolling the pharmacy in the drug distribution management database.

Embodiment 158. The method of any one of embodiments 142-157, further comprising: receiving a distributor enrollment request comprising patient information for the patient; and enrolling the distributor in the drug distribution management database.

Embodiment 159. The method of any one of embodiments 142-158, further comprising: receiving a patient enrollment request comprising patient information for the patient; and enrolling the patient in the drug distribution management database.

Embodiment 160. The method of any one of embodiments 142-159, further comprising: receiving a healthcare setting enrollment request comprising patient information for the patient; and enrolling the healthcare setting in the drug distribution management database.

Embodiment 161. The method of any one of embodiments 142-160, further comprising: receiving a healthcare provider enrollment request comprising patient information for the patient; and enrolling the healthcare provider in the drug distribution management database.

1-73. (canceled)

74. A method of treating a patient for a food allergy by oral immunotherapy, comprising:
(a) enrolling the patient or verifying enrollment of the patient in a drug distribution management database for a drug comprising a food allergen;
(b) administering to the enrolled patient, in a healthcare setting enrolled in the drug distribution management database, a dose of the drug at a first up-dosing level according to an up-dosing schedule;
(c) monitoring the enrolled patient, in the enrolled healthcare setting, for an allergic response to the drug after administering the dose of the drug in step (b);
(d) providing a first prescription requesting a pharmacy enrolled in the drug distribution management database release to the enrolled patient or the enrolled healthcare setting a plurality of doses of the drug at the first up-dosing level;
(e) administering to the enrolled patient, in the enrolled healthcare setting, a dose of the drug at a subsequent up-dosing level according to the up-dosing schedule;
(f) monitoring the enrolled patient, in the enrolled healthcare setting, for an allergic response to the drug after administering the dose of the drug in step (e); and
(g) providing a subsequent prescription requesting the enrolled pharmacy release to the enrolled patient or the enrolled healthcare setting a plurality of doses of the drug at the subsequent up-dosing level.

75. The method of claim 74, wherein the dose of (b) or (e) is administered under the supervision of a healthcare provider enrolled in the drug distribution management database.

76. The method of claim 74, wherein the monitoring of (c) or (f) is under the supervision of a healthcare provider enrolled in the drug distribution management database.

77. The method of claim 74, comprising obtaining the dose of the drug administered to the enrolled patient in step (b) or step (e) from an oral immunotherapy treatment kit comprising a plurality of doses of the drug at different up-dosing levels.

78. The method of claim 77, wherein the oral immunotherapy treatment kit is received from a distributor enrolled in the drug distribution management database.

79. The method of claim 78, comprising submitting a request to the enrolled distributor to provide the oral immunotherapy treatment kit to the enrolled healthcare setting, wherein providing the oral immunotherapy treatment kit to the enrolled healthcare setting is conditioned on the enrolled distributor verifying enrollment of the healthcare setting.

80. The method of claim 74, comprising, prior to step (b), administering to the enrolled patient, in the enrolled healthcare setting, a plurality of different doses of the drug according to an initial dose escalation schedule.

81. The method of claim 80, wherein the plurality of different doses of the drug according to the initial dose escalation schedule are administered under the supervision of a healthcare provider enrolled in the drug distribution management database.

82. The method of claim 80, further comprising monitoring the enrolled patient, in the enrolled healthcare setting, for an allergic response to the drug after administering each different dose of the drug according to the initial dose escalation schedule.

83. The method of claim 82, wherein the monitoring after administering each different dose of the drug according to the initial dose escalation schedule is under the supervision of a healthcare provider enrolled in the drug distribution management database.

84. The method of claim 80, wherein the plurality of different doses of the drug according to the initial dose escalation schedule is obtained from an initial dose escalation kit assigned to the enrolled patient.

85. The method of claim 84, comprising receiving the initial dose escalation kit from the enrolled pharmacy.

86. The method of claim 85, comprising submitting a request to the enrolled pharmacy to provide the initial dose escalation kit to the enrolled healthcare setting, wherein providing the initial dose escalation kit to the enrolled healthcare setting is conditioned on the enrolled pharmacy verifying (1) enrollment of the patient in the drug distribution management database and (2) enrollment of the healthcare setting.

87. The method of claim 86, wherein providing the initial dose escalation kit to the enrolled healthcare setting is further conditioned on the enrolled pharmacy verifying enrollment of a healthcare provider in the drug distribution management database, wherein the healthcare provider is associated with the enrolled patient.

88. A method of initiating treatment of a patient for a food allergy by oral immunotherapy, comprising:
(a) enrolling the patient or verifying enrollment of the patient in a drug distribution management database for a drug comprising a food allergen;
(b) administering to the enrolled patient, in the enrolled healthcare setting, a plurality of different doses of the drug according to an initial dose escalation schedule; and
(c) monitoring the enrolled patient, in the enrolled healthcare setting, for an allergic response to the drug after administering each different dose of the drug according to the initial dose escalation schedule in step (b).

89. The method of claim 88, wherein the plurality of different doses of step (b) are administered under the supervision of a healthcare provider enrolled in the drug distribution management database.

90. The method of claim 88, wherein the monitoring of step (c) is under the supervision of a healthcare provider enrolled in the drug distribution management database.

91. The method of claim 88, wherein the plurality of different doses of the drug according to the initial dose escalation schedule are obtained from an initial dose escalation kit assigned to the enrolled patient.

92. The method of claim 91, comprising receiving the initial dose escalation kit from a pharmacy enrolled in the drug distribution management database.

93. The method of claim 92, comprising submitting a request to the enrolled pharmacy to provide the initial dose escalation kit to the enrolled healthcare setting, wherein providing the initial dose escalation kit to the enrolled healthcare setting is conditioned on the enrolled pharmacy verifying (1) enrollment of the patient in the drug distribution management database and (2) enrollment of the healthcare setting.

94. The method of claim 93, wherein providing the initial dose escalation kit to the enrolled healthcare setting is further conditioned on the enrolled pharmacy verifying enrollment of a healthcare provider in the drug distribution management database, wherein the healthcare provider is associated with the enrolled patient.

95-102. (canceled)

103. The method of claim 88, comprising submitting an enrollment request for the healthcare setting, and receiving an enrollment verification for the healthcare setting.

104. The method of claim 103, wherein the enrollment request affirms that the healthcare setting is equipped to identify and treat a systemic allergic reaction.

105. The method of claim 103, wherein the enrollment request affirms that the healthcare setting will monitor the patient for an allergic response to the drug after administering the drug to the patient.

106. The method of claim 103, wherein the enrollment request affirms that the healthcare setting will, for every patient to be treated with the drug by the healthcare setting, enroll the patient in or verify enrollment of the patient in the drug distribution management database.

107. The method of c1aim 103, wherein the enrollment request affirms that the healthcare setting will only use an initial dose escalation kit for initiating treatment of a patient uniquely associated with the initial dose escalation kit.

108. The method of claim 74, comprising generating a record for each dose of the drug administered to the enrolled patient in the enrolled healthcare setting, wherein the record identifies the administration of the drug and the monitoring of the enrolled patient following administration of the drug.

109. The method of claim 88, further comprising recommending to the patient that the patient have an anaphylaxis-response treatment immediately available to the patient during the course of treatment.

110. The method of claim 109, wherein the anaphylaxis response treatment comprises epinephrine.

111. The method of claim 109, comprising generating a record confirming that the recommendation that the patient have an anaphylaxis-response treatment immediately available to the patient during the course of treatment has been made to the patient.

112. The method of claim 88, further comprising recommending to the patient that the patient avoid dietary consumption of the food during the course of treatment.

113. The method of claim 112, comprising generating a record confirming that the recommendation that the patient avoid dietary consumption of the food during the course of treatment has been made to the patient.

114. The method of claim 88, comprising providing a patient status to a drug distribution management database manager upon discontinuation of treatment by the patient or transfer of care of the patient to a subsequent healthcare setting.

115. (canceled)

116. A method of providing a drug comprising a food allergen to a healthcare setting, comprising:
(a) receiving, at a pharmacy enrolled in a drug distribution management database for the drug, a request to provide to a healthcare setting an initial dose escalation kit associated with an identified patient, the initial dose escalation kit comprising a plurality of different doses of the drug according to an initial dose escalation schedule;
(b) verifying enrollment of the identified patient in a drug distribution management database for the drug;
(c) verifying enrollment of the healthcare setting in the drug distribution management database; and
(d) providing, to the healthcare setting, the initial dose escalation kit associated with the identified patient.

117. (canceled)

118. The method of claim 116, comprising submitting an enrollment request for the pharmacy, and receiving an enrollment verification for the pharmacy.

119. The method of claim 116, comprising generating a record for the patient that associates a treatment level of the provided plurality of doses with the identified patient, wherein the treatment level indicates an initial dose escalation level.

120-141. (canceled)

142. A method for authorizing the provision of a drug comprising a food allergen to a healthcare setting, comprising:
(a) verifying enrollment of the healthcare setting in a drug distribution management database;
(b) verifying enrollment of a patient in the drug distribution management database;
(c) authorizing, after enrollment of the healthcare setting and the patient in the drug distribution management database has been verified, a pharmacy to provide to the healthcare setting an initial dose escalation kit associated with the patient, the initial dose escalation kit comprising a plurality of different doses of the drug according to an initial dose escalation schedule.

143. The method of claim 142, further comprising verifying enrollment of a healthcare provider in the drug distribution management database, wherein step (c) occurs after enrollment of the healthcare provider in the drug distribution management database has been verified.

144-156. (canceled)

157. The method of claim 142, further comprising:
receiving a pharmacy enrollment request comprising patient information for the patient; and
enrolling the pharmacy in the drug distribution management database.

158. The method of claim 142, further comprising:
receiving a distributor enrollment request comprising patient information for the patient; and
enrolling the distributor in the drug distribution management database.

159. The method of claim 142, further comprising:
receiving a patient enrollment request comprising patient information for the patient; and
enrolling the patient in the drug distribution management database.

160. The method of claim 142, further comprising:
receiving a healthcare setting enrollment request comprising patient information for the patient; and
enrolling the healthcare setting in the drug distribution management database.

161. The method of claim 142, further comprising:
receiving a healthcare provider enrollment request comprising patient information for the patient; and
enrolling the healthcare provider in the drug distribution management database.
